SEQUENCE LISTING (91 sequences)

SEQ ID NO: 1 - protein, 802 aa, Homo sapiens
MEEEGGSSGGAAGTSADGGDGGEQLLTVKHELRTANLTGHAEKVGIENFELLKVLGTGAYGKVFLVRKISGHDTGKLYAMKVLKKATIVQKAKTTEHTRTERQVLEHIRQSPFLVTLHYAFQTETKLHLILDYINGGELFTHLSQRERFTEHEVQIYVGEIVLALEHLHKLGIIYRDIKLENILLDSNGHVVLTDFGLSKEFVADETERAYSFCGTIEYMAPDIVRGGDSGHDKAVDWWSLGVLMYELLTGASPFTVDGEKNSQAEISRRILKSEPPYPQEMSALAKDLIQRLLMKDPKKRLGCGPRDADEIKEHLFFQKINWDDLAAKKVPAPFKPVIRDELDVSNFAEEFTEMDPTYSPAALPQSSEKLFQGYSFVAPSILFKRNAAVIDPLQFHMGVERPGVTNVARSAMMKDSPFYQHYDLDLKDKPLGEGSFSICRKCVHKKSNQAFAVKIISKRMEANTQKEITALKLCEGHPNIVKLHEVFHDQLHTFLVMELLNGGELFERIKKKKHFSETEASYIMRKLVSAVSHMHDVGVVHRDLKPENLLFTDENDNLEIKIIDFGFARLKPPDNQPLKTPCFTLHYAAPELLNQNGYDESCDLWSLGVILYTMLSGQVPFQSHDRSLTCTSAVEIMKKIKKGDFSFEGEAWKNVSQEAKDLIQGLLTVDPNKRLKMSGLRYNEWLQDGSQLSSNPLMTPDILGSSGAAVHTCVKATFHAFNKYKREGFCLQNVDKAPLAKRRKMKKTSTSTETRSSSSESSHSSSSHSHGKTTPTKTLQPSNPADSNNPETLFQFSDSVA

SEQ ID NO: 2 - protein, 633 aa, Homo sapiens
MPEIRLRHVVSCSSQDSTHCAENLLKADTYRKWRAAKAGEKTISVVLQLEKEEQIHSVDIGNDGSAFVEVLVGSSAGGAGEQDYEVLLVTSSFMSPSESRSGSNPNRVRMFGPDKLVRAAAEKRWDRVKIVCSQPYSKDSPFGLSFVRFHSPPDKDEAEAPSQKVTVTKLGQFRVKEEDESANSLRPGALFFSRINKTSPVTASDPAGPSYAAATLQASSAASSASPVSRAIGSTSKPQESPKGKRKLDLNQEEKKTPSKPPAQLSPSVPKRPKLPAPTRTPATAPVPARAQGAVTGKPRGEGTEPRRPRAGPEELGKILQGVVVVLSGFQNPFRSELRDKALELGAKYRPDWTRDSTHLICAFANTPKYSQVLGLGGRIVRKEWVLDCHRMRRRLPSRRYLMAGPGSSSEEDEASHSGGSGDEAPKLPQKQPQTKTKPTQAAGPSSPQKPPTPEETKAASPVLQEDIDIEGVQSEGQDNGAEDSGDTEDELRRVAEQKEHRLPPGQEENGEDPYAGSTDENTDSEEHQEPPDLPVPELPDFFQGKHFFLYGEFPGDERRKLIRYVTAFNGELEDNMSDRVQFVITAQEWDPSFEEALMDNPSLAFVRPRWIYSCNEKQKLLPHQLYGVVPQA

SEQ ID NO: 3 - protein, 380 aa, Homo sapiens
MKLLARALRLCEFGRQASSRRLVAGQGCVGPRRGCCAPVQVVGPRADLPPCGACITGRIMRPDDANVAGNVHGGTILKMIEEAGAIISTRHCNSQNGERCVAALARVERTDFLSPMCIGEVAHVSAEITYTSKHSVEVQVNVMSENILTGAKKLTNKATLWYVPLSLKNVDKVLEVPPVVYSRQEQEEEGRKRYEAQKLERMETKWRNGDIVQPVLNPEPNTVSYSQSSLIHLVGPSDCTLHGFVHGGVTMKLMDEVAGIVAARHCKTNIVTASVDAINFHDKIRKGCVITISGRMTFTSNKSMEIEVLVDADPVVDSSQKRYRAASAFFTYVSLSQEGRSLPVPQLVPETEDEKKRFEEGKGRYLQMKAKRQGHAEPQP

SEQ ID NO: 4 - protein, 675 aa, Homo sapiens
MDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIWELKREEITLLKELGSGQFGVVQLGKWKGQYDVAVKMIKEGSMSEDEFFQEAQTMMKLSHPKLVKFYGVCSKEYPIYIVTEYISNGCLLNYLRSHGKGLEPSQLLEMCYDVCEGMAFLESHQFIHRDLAARNCLVDRDLCVKVSDFGMTRYVLDDQYVSSVGTKFPVKWSAPEVFHYFKYSSKSDVWAFGILMWEVFSLGKQPYDLYDNSQVVLKVSQGHRLYRPHLASDTIYQIMYSCWHELPEKRPTFQQLLSSIEPLREKDKH

SEQ ID NO: 5 - protein, 329 aa, Homo sapiens
MWGLKVLLLPVVSFALYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 6 - protein, 285 aa, Homo sapiens
MMEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 7 - protein, 986 aa, Homo sapiens
MSRIESLTRARIDRSRELASKTREKEKMKEAKDARYTNGHLFTTISVSGMTMCYACNKSITAKEALICPTCNVTIHNRCKDTLANCTKVKQKQQKAALLKNNTALQSVSLRSKTTIRERPSSAIYPSDSFRQSLLGSRRGRSSLSLAKSVSTTNIAGHFNDESPLGLRRILSQSTDSLNMRNRTLSVESLIDEAEVIYSELMSDFEMDEKDFAADSWSLAVDSSFLQQHKKEVMKQQDVIYELIQTELHHVRTLKIMTRLFRTGMLEELHLEPGVVQGLFPCVDELSDIHTRFLSQLLERRRQALCPGSTRNFVIHRLGDLLISQFSGPSAEQMCKTYSEFCSRHSKALKLYKELYARDKRFQQFIRKVTRPAVLKRHGVQECILLVTQRITKYPLLISRILQHSHGIEEERQDLTTALGLVKELLSNVDEGIYQLEKGARLQEIYNRMDPRAQTPVPGKGPFGREELLRRKLIHDGCLLWKTATGRFKDVLVLLMTDVLVFLQEKDQKYIFPTLDKPSVVSLQNLIVRDIANQEKGMFLISAAPPEMYEVHTASRDDRSTWIRVIQQSVRTCPSREDFPLIETEDEAYLRRIKMELQQKDRALVELLREKVGLFAEMTHFQAEEDGGSGMALPTLPRGLFRSESLESPRGERLLQDAIREVEGLKDLLVGPGVELLLTPREPALPLEPDSGGNTSPGVTANGEARTFNGSIELCRADSDSSQRDRNGNQLRSPQEEALQRLVNLYGLLHGLQAAVAQQDTLMEARFPEGPERREKLCRANSRDGEAGRAGAAPVAPEKQATELALLQRQHALLQEELRRCRRLGEERATEAGSLEARLRESEQARALLEREAEEARRQLAALGQTEPLPAEAPWARRPVDPRRRSLPAGDALYLSFNPPQPSRGTDRLDLPVTTRSVHRNFEDRERQELGSPEERLQDSSDPDTGSEEEGSSRLSPPHSPRDFTRMQDIPEETESRDGEAVASES

SEQ ID NO: 8 - protein, 404 aa, Homo sapiens
MPYVDRQNRICGFLDIEENENSGKFLRRYFILDTREDSFVWYMDNPQNLPSGSSRVGAIKLTYISKVSDATKLRPKAEFCFVMNAGMRKYFLQANDQQDLVEWVNVLNKAIKITVPKQSDSQPNSDNLSRHGECGKKQVSYRTDIVGGVPIITPTQKEEVNECGESIDRNNLKRSQSHLPYFTPKPPQDSAVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHPPGPSESKHAFRPTNAATATSHSTASRSNSLVSTFTMEKRGFYESLAKVKPGNFKVQTVSPREPASKVTEQALLRPQSKNGPQEKDCDLVDLDDASLPVSDV

SEQ ID NO: 9 - protein, 434 aa, Homo sapiens
MAAAAGSCARVAAWGGKLRRGLAVSRQAVRSPGPLAAAVAGAALAGAGAAWHHSRVSVAARDGSFTVSAQKNVEHGIIYIGKPSLRKQRFMQFSSLEHEGEYYMTPRDFLFSVMFEQMERKTSVKKLTKKDIEDTLSGIQTAGCGSTFFRDLGDKGLISYTEYLFLLTILTKPHSGFHVAFKMLDTDGNEMIEKREFFKLQKIISKQDDLMTVKTNETGYQEAIVKEPEINTTLQMRFFGKRGQRKLHYKEFRRFMENLQTEIQEMEFLQFSKGLSFMRKEDFAEWLLFFTNTENKDIYWKNVREKLSAGESISLDEFKSFCHFTTHLEDFAIAMQMFSLAHRPVRLAEFKRAVKVATGQELSNNILDTVFKIFDLDGDECLSHEEFLGVLKNRMHRGLWVPQHQSIQEYWKCVKKESIKGVKEVWKQAGKGLF

SEQ ID NO: 10 - protein, 346 aa, Homo sapiens
MVLVHVGYLVLPVFGSVRNRGAPFQRSQHPHATSCRHFHLGPPQPQQLAPDFPLAHPVQSQPGLSAHMAPAHQHSGALHQSLTPLPTLQFQDVTGPSFLPQALHQQYLLQQQLLEAQHRRLVSHPRRSQERVSVHPHRLHPSFDFGQLQTPQPRYLAEGTDWDLSVDAGLSPAQFQVRPIPQHYQHYLATPRMHHFPRNSSSTQMVVHEIRNYPYPQLHFLALQGLNPSRHTSAVRESYEELLQLEDRLGNVTRGAVQNTIERFTFPHKYKKRRPQDGKGKKDEGEESDTDEKCTICLSMLEDGEDVRRLPCMHLFHQLCVDQWLAMSKKCPICRVDIETQLGADS

SEQ ID NO: 11 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 12 - protein, 785 aa, Homo sapiens
MVVPSLKLQDLIEEIRGAKTQAQEREVIQKECAHIRASFRDGDPVHRHRQLAKLLYVHMLGYPAHFGQMECLKLIASSRFTDKRVGYLGAMLLLDERHDAHLLITNSIKNDLSQGIQPVQGLALCTLSTMGSAEMCRDLAPEVEKLLLQPSPYVRKKAILTAVHMIRKVPELSSVFLPPCAQLLHERHHGILLGTITLITELCERSPAALRHFRKVVPQLVHILRTLVTMGYSTEHSISGVSDPFLQVQILRLLRILGRNHEESSETMNDLLAQVATNTDTSRNAGNAVLFETVLTIMDIRSAAGLRVLAVNILGRFLLNSDRNIRYVALTSLLRLVQSDHSAVQRHRPTVVECLRETDASLSRRALELSLALVNSSNVRAMMQELQAFLESCPPDLRADCASGILLAAERFAPTKRWHIDTILHVLTTAGTHVRDDAVANLTQLIGGAQELHAYSVRRLYNALAEDISQQPLVQVAAWCIGEYGDLLLAGNCEEIEPLQVDEEEVLALLEKVLQSHMSLPATRGYALTALMKLSTRLCGDNNRIRQVVSIYGSCLDVELQQRAVEYDTLFRKYDHMRAAILEKMPLVERDGPQADEEAKESKEAAQLSEAAPVPTEPQASQLLDLLDLLDGASGDVQHPPHLDPSPGGALVHLLDLPCVPPPPAPIPDLKVFEREGVQLNLSFIRPPENPALLLITITATNFSEGDVTHFICQAAVPKSLQLQLQAPSGNTVPARGGLPITQLFRILNPNKAPLRLKLRLTYDHFHQSVQEIFEVNNLPVESWQ

SEQ ID NO: 13 - protein, 329 aa, Homo sapiens
MVRTKTWTLKKHFVGYPTNSDFELKTAELPPLKNGEVLLEALFLTVDPYMRVAAKRLKEGDTMMGQQVAKVVESKNVALPKGTIVLASPGWTTHSISDGKDLEKLLTEWPDTIPLSLALGTVGMPGLTAYFGLLEICGVKGGETVMVNAAAGAVGSVVGQIAKLKGCKVVGAVGSDEKVAYLQKLGFDVVFNYKTVESLEETLKKASPDGYDCYFDNVGGEFSNTVIGQMKKFGRIAICGAISTYNRTGPLPPGPPPEIVIYQELRMEAFVVYRWQGDARQKALKDLLKWVLEGKIQYKEYIIEGFENMPAAFMGMLKGDNLGKTIVKA

SEQ ID NO: 14 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 15 - protein, 358 aa, Homo sapiens
MDDATVLRKKGYIVGINLGKGSYAKVKSAYSERLKFNVAVKIIDRKKTPTDFVERFLPREMDILATVNHGSIIKTYEIFETSDGRIYIIMELGVQGDLLEFIKCQGALHEDVARKMFRQLSSAVKYCHDLDIVHRDLKCENLLLDKDFNIKLSDFGFSKRCLRDSNGRIILSKTFCGSAAYAAPEVLQSIPYQPKVYDIWSLGVILYIMVCGSMPYDDSDIRKMLRIQKEHRVDFPRSKNLTCECKDLIYRMLQPDVSQRLHIDEILSHSWLQPPKPKATSSASFKREGEGKYRAECKLDTKTGLRPDHRPDHKLGAKTQHRLLVVPENENRMEDRLAETSRAKDHHISGAEVGKAST

SEQ ID NO: 16 - protein, 505 aa, Homo sapiens
MGLVSSKKPDKEKPIKEKDKGQWSPLKVSAQDKDAPPLPPLVVFNHLTPPPPDEHLDEDKHFVVALYDYTAMNDRDLQMLKGEKLQVLKGTGDWWLARSLVTGREGYVPSNFVARVESLEMERWFFRSQGRKEAERQLLAPINKAGSFLIRESETNKGAFSLSVKDVTTQGELIKHYKIRCLDEGGYYISPRITFPSLQALVQHYSKKGDGLCQRLTLPCVRPAPQNPWAQDEWEIPRQSLRLVRKLGSGQFGEVWMGYYKNNMKVAIKTLKEGTMSPEAFLGEANVMKALQHERLVRLYAVVTKEPIYIVTEYMARGCLLDFLKTDEGSRLSLPRLIDMSAQIAEGMAYIERMNSIHRDLRAANILVSEALCCKIADFGLARIIDSEYTAQEGAKFPIKWTAPEAIHFGVFTIKADVWSFGVLLMEVVTYGRVPYPGMSNPEVIRNLERGYRMPRPDTCPPELYRGVIAECWRSRPEERPTFEFLQSVLEDFYTATERQYELQP

SEQ ID NO: 17 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 18 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 19 - protein, 540 aa, Homo sapiens
MPFAEDKTYKYICRNFSNFCNVDVVEILPYLPCLTARDQDRLRATCTLSGNRDTLWHLFNTLQRRPGWVEYFIAALRGCELVDLADEVASVYQSYQPRTSDRPPDPLEPPSLPAERPGPPTPAAAHSIPYNSCREKEPSYPMPVQETQAPESPGENSEQALQTLSPRAIPRNPDGGPLESSSDLAALSPLTSSGHQEQDTELGSTHTAGATSSLTPSRGPVSPSVSFQPLARSTPRASRLPGPTGSVVSTGTSFSSSSPGLASAGAAEGKQGAESDQAEPIICSSGAEAPANSLPSKVPTTLMPVNTVALKVPANPASVSTVPSKLPTSSKPPGAVPSNALTNPAPSKLPINSTRAGMVPSKVPTSMVLTKVSASTVPTDGSSRNEETPAAPTPAGATGGSSAWLDSSSENRGLGSELSKPGVLASQVDSPFSGCFEDLAISASTSLGMGPCHGPEENEYKSEGTFGIHVAENPSIQLLEGNPGPPADPDGGPRPQADRKFQEREVPCHRPSPGALWLQVAVTGVLVVTLLVVLYRRRLH

SEQ ID NO: 20 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 21 - protein, 357 aa, Homo sapiens
MQSVQSTSFCLRKQCLCLTFLLLHLLGQVAATQRCPPQCPGRCPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 22 - protein, 82 aa, Homo sapiens
MASSSGAGAAAAAAAANLNAVRETMDVLLEISRILNTGLDMETLSICVRLCEQGINPEALSSVIKELRKATEALKAAENMTS

SEQ ID NO: 23 - protein, 1137 aa, Homo sapiens
MFDCWRFILCKRPGSNSYSSPQRPNEAKKEETDHQIDVSDVIRLVQDTPEATAMATDEIMHQDIVPLCAADIQDQLKKRFAYLSGGRGQDGSPVITFPDYPAFSEIPDKEFQNVMTYLTSIPSLQDAGIGFILVIDRRRDKWTSVKASVLRIAASFPANLQLVLVLRPTGFFQRTLSDIAFKFNRDDFKMKVPVIMLSSVPDLHGYIDKSQLTEDLGGTLDYCHSRWLCQRTAIESFALMVKQTAQMLQSFGTELAETELPNDVQSTSSVLCAHTEKKDKAKEDLRLALKEGHSVLESLRELQAEGSEPSVNQDQLDNQATVQRLLAQLNETEAAFDEFWAKHQQKLEQCLQLRHFEQGFREVKAILDAASQKIATFTDIGNSLAHVEHLLRDLASFEEKSGVAVERARALSLDGEQLIGNKHYAVDSIRPKCQELRHLCDQFSAEIARRRGLLSKSLELHRRLETSMKWCDEGIYLLASQPVDKCQSQDGAEAALQEIEKFLETGAENKIQELNAIYKEYESILNQDLMEHVRKVFQKQASMEEVFHRRQASLKKLAARQTRPVQPVAPRPEALAKSPCPSPGIRRGSENSSSEGGALRRGPYRRAKSEMSESRQGRGSAGEEEESLAILRRHVMSELLDTERAYVEELLCVLEGYAAEMDNPLMAHLLSTGLHNKKDVLFGNMEEIYHFHNRIFLRELENYTDCPELVGRCFLERMEDFQIYEKYCQNKPRSESLWRQCSDCPFFQECQRKLDHKLSLDSYLLKPVQRITKYQLLLKEMLKYSRNCEGAEDLQEALSSILGILKAVNDSMHLIAITGYDGNLGDLGKLLMQGSFSVWTDHKRGHTKVKELARFKPMQRHLFLHEKAVLFCKKREENGEGYEKAPSYSYKQSLNMAAVGITENVKGDAKKFEIWYNAREEVYIVQAPTPEIKAAWVNEIRKVLTSQLQACREASQHRALEQSQSLPLPAPTSTSPSRGNSRNIKKLEERKTDPLSLEGYVSSAPLTKPPEKGKGWSKTSHSLEAPEDDGGWSSAEEQINSSDAEEDGGLGPKKLVPGKYTVVADHEKGGPDALRVRSGDVVELVQEGDEGLWYVRDPTTGKEGWVPASSLSVRLGPSGSAQCLSSSGKAHVPRAHP

SEQ ID NO: 24 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 25 - protein, 277 aa, Homo sapiens
MSSGIHVALVTGGNKGIGLAIVRDLCRLFSGDVVLTARDVTRGQAAVQQLQAEGLSPRFHQLDIDDLQSIRALRDFLRKEYGGLDVLVNNAGIAFKVADPTPFHIQAEVTMKTNFFGTRDVCTELLPLIKPQGRVVNVSSIMSVRALKSCSPELQQKFRSETITEEELVGLMNKFVEDTKKGVHQKEGWPSSAYGVTKIGVTVLSRIHARKLSEQRKGDKILLNACCPGWVRTDMAGPKATKSPEEGAETPVYLALLPPDAEGPHGQFVSEKRVEQW

SEQ ID NO: 26 - protein, 187 aa, Homo sapiens
MRQGLLVLALVLVLVLVLAAGSQVQEWYPRESHALNWNKFSGFWYILATATDAQGFLPARDKRKLGASVVKVNKVGQLRVLLAFRRGQGCGRAQPRHPGTSGHLWASLSVKGVKAFHVLSTDYSYGLVYLRLGRATQNYKNLLLFHRQNVSSFQSLKEFMDACDILGLSKAAVILPKDASRTHTILP

SEQ ID NO: 27 - protein, 950 aa, Homo sapiens
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 28 - protein, 217 aa, Homo sapiens
MAYPGYGGGFGNFSIQVPGMQMGQPVPETGPAILLDGYSGPAYSDTYSSAGDSVYTYFSAVAGQDGEVDAEELQRCLTQSGINGTYSPFSLETCRIMIAMLDRDHTGKMGFNAFKELWAALNAWKENFMTVDQDGSGTVEHHELRQAIGLMGYRLSPQTLTTIVKRYSKNGRIFFDDYVACCVKLRALTDFFRKRDHLQQGSANFIYDDFLQGTMAI

SEQ ID NO: 29 - protein, 558 aa, Homo sapiens
MRKIRANAIAILTVAWILGTFYYLWQDNRAHAASSGGRGAQRAGRRSEQLREDRTIPLIVTGTPSKGFDEKAYLSAKQLKAGEDPYRQHAFNQLESDKLSPDRPIRDTRHYSCPSVSYSSDLPATSVIITFHNEARSTLLRTVKSVLNRTPANLIQEIILVDDFSSDPEDCLLLTRIPKVKCLRNDRREGLIRSRVRGADVAAATVLTFLDSHCEVNTEWLPPMLQRVKEDHTRVVSPIIDVISLDNFAYLAASADLRGGFDWSLHFKWEQIPLEQKMTRTDPTRPIRTPVIAGGIFVIDKSWFNHLGKYDAQMDIWGGENFELSFRVWMCGGSLEIVPCSRVGHVFRKRHPYNFPEGNALTYIRNTKRTAEVWMDEYKQYYYEARPSAIGKAFGSVATRIEQRKKMNCKSFRWYLENVYPELTVPVKEALPGIIKQGVNCLESQGQNTAGDFLLGMGICRGSAKNPQPAQAWLFSDHLIQQQGKCLAATSTLMSSPGSPVILQMCNPREGKQKWRRKGSFIQHSVSGLCLETKPAQLVTSKCQADAQAQQWQLLPHT

SEQ ID NO: 30 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 31 - protein, 234 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 32 - protein, 648 aa, Homo sapiens [sequence omitted]

SEQ ID NO: 33 - protein, 617 aa, Homo sapiens
MRCLAARVNYKTLIIICALFTLVTVLLWNKCSSDKAIQFPRRSSSGFRVDGFEKRAAASESNNYMNHVAKQQSEEAFPQEQQKAPPVVGGFNSNVGSKVLGLKYEEIDCLINDEHTIKGRREGNEVFLPFTWVEKYFDVYGKVVQYDGYDRFEFSHSYSKVYAQRAPYHPDGVFMSFEGYNVEVRDRVKCISGVEGVPLSTQWGPQGYFYPIQIAQYGLSHYSKNLTEKPPHIEVYETAEDRDKNKPNDWTVPKGCFMANVADKSRFTNVKQFIAPETSEGVSLQLGNTKDFIISFDLKFLTNGSVSVVLETTEKNQLFTIHYVSNAQLIAFKERDIYYGIGPRTSWSTVTRDLVTDLRKGVGLSNTKAVKPTKIMPKKVVRLIAKGKGFLDNITISTTAHMAAFFAASDWLVRNQDEKGGWPIMVTRKLGEGFKSLEPGWYSAMAQGQAISTLVRAYLLTKDHIFLNSALRATAPYKFLSEQHGVKAVFMNKHDWYEEYPTTPSSFVLNGFMYSLIGLYDLKETAGEKLGKEARSLYERGMESLKAMLPLYDTGSGTIYDLRHFMLGIAPNLARWDYHTTHINQLQLLSTIDESPVFKEFVKRWKSYLKGSRAKHN

SEQ ID NO: 34 - protein, 407 aa, Homo sapiens
MTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLD

SEQ ID NO: 35 - protein, 579 aa, Homo sapiens
MEVNAGGVIAYISSSSSASSPASCHSEGSENSFQSSSSSVPSSPNSSNSDTNGNPKNGDLANIEGILKNDRIDCSMKTSKSSAPGMTKSHSGVTKFSGMVLLCKVCGDVASGFHYGVHACEGCKGFFRRSIQQNIQYKKCLKNENCSIMRMNRNRCQQCRFKKCLSVGMSRDAVRFGRIPKREKQRMLIEMQSAMKTMMNSQFSGHLQNDTLVEHHEQTALPAQEQLRPKPQLEQENIKSSSPPSSDFAKEEVIGMVTRAHKDTFMYNQEQQENSAESMQPQRGERIPKNMEQYNLNHDHCGNGLSSHFPCSESQQHLNGQFKGRNIMHYPNGHAICIANGHCMNFSNAYTQRVCDRVPIDGFSQNENKNSYLCNTGGRMHLVCPLSKSPYVDPHKSGHEIWEEFSMSFTPAVKEVVEFAKRIPGFRDLSQHDQVNLLKAGTFEVLMVRFASLFDAKERTVTFLSGKKYSVDDLHSMGAGDLLNSMFEFSEKLNALQLSDEEMSLFTAVVLVSADRSGIENVNSVEALQETLIRALRTLIMKNHPNEASIFTKLLLKLPDLRSLNNMHSEELLAFKVHP

SEQ ID NO: 36 - protein, 205 aa, Homo sapiens
MASDHQTQAGKPQSLNPKIIIFEQENFQGHSHELNGPCPNLKETGVEKAGSVLVQAGPWVGYEQANCKGEQFVFEKGEYPRWDSWTSSRRTDSLSSLRPIKVDSQEHKIILYENPNFTGKKMEIIDDDVPSFHAHGYQEKVSSVRVQSGTWVGYQYPGYRGLQYLLEKGDYKDSSDFGAPHPQVQSVRRIRDMQWHQRGAFHPSN

SEQ ID NO: 37 - protein, 821 aa, Homo sapiens
MAANGDSPPWSPALAAEGRGSSCEVRRERTPEARIHSVKRYPDLSPGPKGRSSADHAALNSIVSLQASVSFEDVTVDFSKEEWQHLDPAQRRLYWDVTLENYSHLLSVGYQIPKSEAAFKLEQGEGPWMLEGEAPHQSCSGEAIGKMQQQGIPGGIFFHCERFDQPIGEDSLCSILEELWQDNDQLEQRQENQNNLLSHVKVLIKERGYEHKNIEKIIHVTTKLVPSIKRLHNCDTILKHTLNSHNHNRNSATKNLGKIFGNGNNFPHSPSSTKNENAKTGANSCEHDHYEKHLSHKQAPTHHQKIHPEEKLYVCTECVMGFTQKSHLFEHQRIHAGEKSRECDKSNKVFPQKPQVDVHPSVYTGEKPYLCTQCGKVFTLKSNLITHQKIHTGQKPYKCSECGKAFFQRSDLFRHLRIHTGEKPYECSECGKGFSQNSDLSIHQKTHTGEKHYECNECGKAFTRKSALRMHQRIHTGEKPYVCADCGKAFIQKSHFNTHQRIHTGEKPYECSDCGKSFTKKSQLHVHQRIHTGEKPYICTECGKVFTHRTNLTTHQKTHTGEKPYMCAECGKAFTDQSNLIKHQKTHTGEKPYKCNGCGKAFIWKSRLKIHQKSHIGERHYECKDCGKAFIQKSTLSVHQRIHTGEKPYVCPECGKAFIQKSHFIAHHRIHTGEKPYECSDCGKCFTKKSQLRVHQKIHTGEKPNICAECGKAFTDRSNLITHQKIHTREKPYECGDCGKTFTWKSRLNIHQKSHTGERHYECSKCGKAFIQKATLSMHQIIHTGKKPYACTECQKAFTDRSNLIKHQKMHSGEKRYKASD

SEQ ID NO: 38 - protein, 428 aa, Homo sapiens
MDPSVTLWQFLLQLLREQGNGHIISWTSRDGGEFKLVDAEEVARLWGLRKNKTNMNYDKLSRALRYYYDKNIIRKVSGQKFVYKFVSYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPSAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSPGPQKP

SEQ ID NO: 39 - protein, 201 aa, Homo sapiens
MGNVMEGKSVEELSSTECHQWYKKFMTECPSGQLTLYEFRQFFGLKNLSPSASQYVEQMFETFDFNKDGYIDFMEYVAALSLVLKGKVEQKLRWYFKLYDVDGNGCIDRDELLTIIQAIRAINPCSDTTMTAEEFTDTVFSKIDVNGDGELSLEEFIEGVQKDQMLLDTLTRSLDLTRIVRRLQNGEQDEEGADEAAEAAG

SEQ ID NO: 40 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 41 - protein, 450 aa, Homo sapiens
MEGSKTSNNSTMQVSFVCQRCSQPLKLDTSFKILDRVTIQELTAPLLTTAQAKPGETQEEETNSGEEPFIETPRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQMELKELALEEERLIQELEDVEKNRKIVAENLEKVQAEAERLDQEEAQYQREYSEFKRQQLELDDELKSVENQMRYAQTQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 42 - protein, 901 aa, Homo sapiens
MEPPLPVGAQPLATVEGMEMKGPLREPCALTLAQRNGQYELIIQLHEKEQHVQDIIPINSHFRCVQEAEETLLIDIASNSGCKIRVQGDWIRERRFEIPDEEHCLKFLSAVLAAQKAQSQLLVPEQKDSSSWYQKLDTKDKPSVFSGLLGFEDNFSSMNLDKKINSQNQPTGIHREPPPPPFSVNKMLPREKEASNKEQPKVTNTMRKLFVPNTQSGQREGLIKHILAKREKEYVNIQTFRFFVGTWNVNGQSPDSGLEPWLNCDPNPPDIYCIGFQELDLSTEAFFYFESVKEQEWSMAVERGLHSKAKYKKVQLVRLVGMMLLIFARKDQCRYIRDIATETVGTGIMGKMGNKGGVAVRFVFHNTTFCIVNSHLAAHVEDFERRNQDYKDICARMSFVVPNQTLPQLNIMKHEVVIWLGDLNYRLCMPDANEVKSLINKKDLQRLLKFDQLNIQRTQKKAFVDFNEGEIKFIPTYKYDSKTDRWDSSGKCRVPAWCDRILWRGTNVNQLNYRSHMELKTSDHKPVSALFHIGVKVVDERRYRKVFEDSVRIMDRMENDFLPSLELSRREFVFENVKFRQLQKEKFQISNNGQVPCHFSFIPKLNDSQYCKPWLRAEPFEGYLEPNETVDISLDVYVSKDSVTILNSGEDKIEDILVLHLDRGKDYFLTISGNYLPSCFGTSLEALCRMKRPIREVPVTKLIDLEEDSFLEKEKSLLQMVPLDEGASERPLQVPKEIWLLVDHLFKYACHQEDLFQTPGMQEELQQIIDCLDTSIPETIPGSNHSVAEALLIFLEALPEPVICYELYQRCLDSAYDPRICRQVISQLPRCHRNVFRYLMAFLRELLKFSEYNSVNANMIATLFTSLLLRPPPNLMARQTPSDRQRAIQFLLGFLLGSEED

SEQ ID NO: 43 - protein, 113 aa, Homo sapiens
MEDYQAAEETAFVVDEVSNIVKEAIESAIGGNAYQHSKVNQWTTNVVEQTLSQLTKLGKPFKYIVTCVIMQKNGAGLHTASSCFWDSSTDGSCTVRWENKTMYCIVSAFGLSI

SEQ ID NO: 44 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 45 - protein, 94 aa, Homo sapiens
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 46 - protein, 263 aa, Homo sapiens
MFYFHCPPQLEGTATFGNHSSGDFDDGFLRRKQRRNRTTFTLQQLEALEAVFAQTHYPDVFTREELAMKINLTEARVQVWFQNRRAKWRKTERGASDQEPGAKEPMAEVTPPPVRNINSPPPGDQARSKKEALEAQQSLGRTVGPAGPFFPSCLPGTLLNTATYAQALSHVASLKGGPLCSCCVPDPMGLSFLPTYGCQSNRTASVATLRMKAREHSEAVLQSANLLPSTSSSPGPVAKPAPPDGSQEKTSPTKEQSEAEKSV

SEQ ID NO: 47 - protein, 267 aa, Homo sapiens
MARFLTLCTWLLLLGPGLLATVRAECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 48 - protein, 655 aa, Homo sapiens
MVAPKSHTDDWAPGPFSSKPQRSQLQIFSSVLQTSLLFLLMGLRASGKDSAPTVVSGILGGSVTLPLNISVDTEIENVIWIGPKNALAFARPKENVTIMVKSYLGRLDITKWSYSLCISNLTLNDAGSYKAQINQRNFEVTTEEEFTLFVYEQLQEPQVTMKSVKVSENFSCNITLMCSVKGAEKSVLYSWTPREPHASESNGGSILTVSRTPCDPDLPYICTAQNPVSQRSSLPVHVGQFCTDPGASRGGTTGETVVGVLGEPVTLPLALPACRDTEKVVWLFNTSIISKEREEAATADPLIKSRDPYKNRVWVSSQDCSLKISQLKIEDAGPYHAYVCSEASSVTSMTHVTLLIYRRLRKPKITWSLRHSEDGICRISLTCSVEDGGNTVMYTWTPLQKEAVVSQGESHLNVSWRSSENHPNLTCTASNPVSRSSHQFLSENICSGPERNTKLWIGLFLMVCLLCVGIFSWCIWKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 49 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 50 - protein, 734 aa, Homo sapiens
MRPAVLGSPDRAPPEDEGPVMVKLEDSEEEGEAALWDPGPEAARLRFRCFRYEEATGPQEALAQLRELCRQWLRPEVRSKEQMLELLVLEQFLGALPPEIQARVQGQRPGSPEEAAALVDGLRREPGGPRRWVTVQVQGQEVLSEKMEPSSFQPLPETEPPTPEPGPKTPPRTMQESPLGLQVKEESEVTEDSDFLESGPLAATQESVPTLLPEEAQRCGTVLDQIFPHSKTGPEGPSWREHPRALWHEEAGGIFSPGFALQLGSISAGPGSVSPHLHVPWDLGMAGLSGQIQSPSREGGFAHALLLPSDLRSEQDPTDEDPCRGVGPALITTRWRSPRGRSRGRPSTGGGVVRGGRCDVCGKVFSQRSNLLRHQKIHTGERPFVCSECGRSFSRSSHLLRHQLTHTEERPFVCGDCGQGFVRSARLEEHRRVHTGEQPFRCAECGQSFRQRSNLLQHQRIHGDPPGPGAKPPAPPGAPEPPGPFPCSECRESFARRAVLLEHQAVHTGDKSFGCVECGERFGRRSVLLQHRRVHSGERPFACAECGQSFRQRSNLTQHRRIHTGERPFACAECGKAFRQRPTLTQHLRVHTGEKPFACPECGQRFSQRLKLTRHQRTHTGEKPYHCGECGLGFTQVSRLTEHQRIHTGERPFACPECGQSFRQHANLTQHRRIHTGERPYACPECGKAFRQRPTLTQHLRTHRREKPFACQDCGRRFHQSTKLIQHQRVHSAE

SEQ ID NO: 51 - protein, 220 aa, Homo sapiens
MERYAGALEEVADGARQQERHYQLLSALQSLVKELPSSFQQRLSYTTLSDLALALLDGTVFEIVQGLLEIQHLTEKSLYNQRLRLQNEHRVLRQALRQKHQEAQQACRPHNLPVLQAAQQRELEAVEHRIREEQRAMDQKIVLELDRKVADQQSTLEKAGVAGFYVTTNPQELMLQMNLLELIRKLQQRGCWAGKAALGLGGPWQLPAAQCDQKGSPVPP

SEQ ID NO: 52 - protein, 448 aa, Homo sapiens
MAHVGDCTQTPWLPVLVVSLMCSARAEYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATALIIAMSTIFIMAIAIVLIIMFYILKTKPSAPACCTSHPGKSVEAQVSKDEEKKEAPDNVVMFSEKDEFEKLTATPAKPTKSENDASSENEQLLSRSVDSDEEPAPDKQGSPELCLLSLVHLAREKSATSNKSAGIQSRRKKILDVYANVCGVVEGLSPTELPFDCLEKTSRMLSSTYNSEKAVVKTWRHLAESFGLKRDEIGGMTDGMQLFDRISTAGYSIPELLTKLVQIERLDAVESLCADILEWAGVVPPASQPHAAS

SEQ ID NO: 53 - protein, 937 aa, Homo sapiens
MVCPNGYDPGGRHLLLFIIILAAWEAGRGQLHYSVPEEAKHGNFVGRIAQDLGLELAELVPRLFRAVCKFRGDLLEVNLQNGILFVNSRIDREELCGRSAECSIHLEVIVERPLQVFHVDVEVKDINDNPPVFPATQRNLFIAESRPLDSRFPLEGASDADIGENALLTYRLSPNEYFFLDVPTSNQQVKPLGLVLRKLLDREETPELHLLLTATDGGKPELTGTVQLLITVLDNNDNAPVFDRTLYTVKLPENVSIGTLVIHPNASDLDEGLNGDIIYSFSSDVSPDIKSKFHMDPLSGAITVIGHMDFEESRAHKIPVEAVDKGFPPLAGHCTVLVEVVDVNDNAPQLTLTSLSLPIPEDAQPGTVITLISVFDRDFGVNGQVTCSLTPRVPFKLVSTFKNYYSLVLDSALDRESVSAYELVVTARDGGSPSLWATASVSVEVADVNDNAPAFAQPEYTVFVKENNPPGCHIFTVSAGDADAQKNALVSYSLVELRVGERALSSYVSVHAESGKVYALQPLDHEELELLQFQVSARDAGVPPLGSNVTLQVFVLDENDNAPALLAPRVGGTGGAVRELVPRSVGAGHVVAKVRAVDADSGYNAWLSYELQPVAAGASIPFRVGLYTGEISTTRALDETDAPRHRLLVLVKDHGEPSLTATATVLVSLVESGQAPKASSRASLGIAGPETELVDVNVYLIIAICAVSSLLVLTLLLYTALRCSAPSSEGACSLVKPTLVCSSAVGSWSFSQQRRQRVCSGEGPPKTDLMAFSPSLPQGPSSTDNPRQPNPDWRYSASLRAGMHSSVHLEEAGILRAGPGGPDQQWPTVSSATPEPEAGEVSPPVGAGVNSNSWTFKYGPGNPKQSGPGELPDKFIIPGSPAIISIRQEPTNSQIDKSDFITFGKKEETKKKKKKKKGNKTQEKKEKGNSTTDNSDQ

SEQ ID NO: 54 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 55 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 56 - protein, 259 aa, Homo sapiens
MAALMRSKDSSCCLLLLAAVLMVESSQIGSSRAKLNSIKSSLGGETPGQAANRSAGMYQGLAFGGSKKGKNLGQAYPCSSDKECEVGRYCHSPHQGSSACMVCRRKKKRCHRDGMCCPSTRCNNGICIPVTESILTPHIPALDGTRHRDRNHGHYSNHDLGWQNLGRPHTKMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 57 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 58 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 59 - protein, 1114 aa, Homo sapiens
MLRGTMTAWRGMRPEVTLACLLLATAGCFADLNEVPQVTVQPASTVQKPGGTVILGCVVEPPRMNVTWRLNGKELNGSDDALGVLITHGTLVITALNNHTVGRYQCVARMPAGAVASVPATVTLANLQDFKLDVQHVIEVDEGNTAVIACHLPESHPKAQVRYSVKQEWLEASRGNYLIMPSGNLQIVNASQEDEGMYKCAAYNPVTQEVKTSGSSDRLRVRRSTAEAARIIYPPEAQTIIVTKGQSLILECVASGIPPPRVTWAKDGSSVTGYNKTRFLLSNLLIDTTSEEDSGTYRCMADNGVGQPGAAVILYNVQVFEPPEVTMELSQLVIPWGQSAKLTCEVRGNPPPSVLWLRNAVPLISSQRLRLSRRALRVLSMGPEDEGVYQCMAENEVGSAHAVVQLRTSRPSITPRLWQDAELATGTPPVSPSKLGNPEQMLRGQPALPRPPTSVGPASPQCPGEKGQGAPAEAPIILSSPRTSKTDSYELVWRPRHEGSGRAPILYYVVKHRKVTNSSDDWTISGIPANQHRLTLTRLDPGSLYEVEMAAYNCAGEGQTAMVTFRTGRRPKPEIMASKEQQIQRDDPGASPQSSSQPDHGRLSPPEAPDRPTISTASETSVYVTWIPRGNGGFPIQSFRVEYKKLKKVGDWILATSAIPPSRLSVEITGLEKGTSYKFRVRALNMLGESEPSAPSRPYVVSGYSGRVYERPVAGPYITFTDAVNETTIMLKWMYIPASNNNTPIHGFYIYYRPTDSDNDSDYKKDMVEGDKYWHSISHLQPETSYDIKMQCFNEGGESEFSNVMICETKARKSSGQPGRLPPPTLAPPQPPLPETIERPVGTGAMVARSSDLPYLIVGVVLGSIVLIIVTFIPFCLWRAWSKQKHTTDLGFPRSALPPSCPYTMVPLGGLPGHQASGQPYLSGISGRACANGIHMNRGCPSAAVGYPGMKPQQHCPGELQQQSDTSSLLRQTHLGNGYDPQSHQITRGPKSSPDEGSFLYTLPDDSTHQLLQPHHDCCQRQEQPAAVGQSGVRRAPDSPVLEAVWDPPFHSGPPCCLGLVPVEEVDSPDSCQVSGGDWCPQHPVGAYVGQEPGMQLSPGPLVRVSFETPPLTI

SEQ ID NO: 60 - protein, 415 aa, Homo sapiens
MAASISGYTFSAVCFHSANSNADHEGFLLGEVRQEETFSISDSQISNTEFLQVIEIHNHQPCSKLFSFYDYASKVNEESLDRILKDRRKKVIGWYRFRRNTQQQMSYREQVLHKQLTRILGVPDLVFLLFSFISTANNSTHALEYVLFRPNRRYNQRISLAIPNLGNTSQQEYKVSSVPNTSQSYAKVIKEHGTDFFDKDGVMKDIRAIYQVYNALQEKVQAVCADVEKSERVVESCQAEVNKLRRQITQRKNEKEQERRLQQAVLSRQMPSESLDPAFSPRMPSSGFAAEGRSTLGDAEASDPPPPYSDFHPNNQESTLSHSRMERSVFMPRPQAVGSSNYASTSAGLKYPGSGADLPPPQRAAGDSGEDSDDSDYENLIDPTEPSNSEYSHSKDSRPMAHPDEDPRNTQTSQI

SEQ ID NO: 61 - protein, 406 aa, Homo sapiens
MHPAVFLSLPDLRCSLLLLVTWVFTPVTTEITSLDTENIDEILNNADVALVNFYADWCRFSQMLHPIFEEASDVIKEEFPNENQVVFARVDCDQHSDIAQRYRISKYPTLKLFRNGMMMKREYRGQRSVKALADYIRQQKSDPIQEIRDLAEITTLDRSKRNIIGYFEQKDSDNYRVFERVANILHDDCAFLSAFGDVSKPERYSGDNIIYKPPGHSAPDMVYLGAMTNFDVTYNWIQDKCVPLVREITFENGEELTEEGLPFLILFHMKEDTESLEIFQNEVARQLISEKGTINFLHADCDKFRHPLLHIQKTPADCPVIAIDSFRHMYVFGDFKDVLIPGKLKQFVFDLHSGKLHREFHHGPDPTDTAPGEQAQDVASSPPESSFQKLAPSEYRYTLLRDRDEL

SEQ ID NO: 62 - protein, 913 aa, Staphylococcus aureus
MKKRIDYLSNKQNKYSIRRFTVGTTSVIVGATILFGIGNHQAQASEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPKDPTPGPPVDPEPSPDPEPEPTPDPEPSPDPEPEPSPDPDPDSDSDSDSGSDSDSGSDSDSESDSDSDSDSDSDSDSDSESDSDSESDSDSDSDSDSDSDSDSDSDSDSDSDSDSDSDSDSESDSDSESDSESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSRVTPPNNEQKAPSNPKGEVNHSNKVSKQHKTDALPETGDKSENTNATLFGAMMALLGSLLLFRKRKQDHKEKA

SEQ ID NO: 63 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 64 - protein, 129 aa, Homo sapiens
MRSLCCAPLLLLLLLPPLLLTPRAGDAAVITGACDKDSQCGGGMCCAVSIWVKSIRICTPMGKLGDSCHPLTRKNNFGNGRQERRKRKRSKRKKEVPFFGRRMHHTCPCLPGLACLRTSFNRFICLAQK

SEQ ID NO: 65 - protein, 520 aa, Homo sapiens
MGKRQHQKDKMYITCAEYTHFYGGKKPDLPQTNFRRLPFDHCSLSLQPFVYPVCTPDGIVFDLLNIVPWLKKYGTNPSNGEKLDGRSLIKLNFSKNSEGKYHCPVLFTVFTNNTHIVAVRTTGNVYAYEAVEQLNIKAKNFRDLLTDEPFSRQDIITLQDPTNLDKFNVSNFYHVKNNMKIIDPDEEKAKQDPSYYLKNTNAETRETLQELYKEFKGDEILAATMKAPEKKKVDKLNAAHYSTGKVSASFTSTAMVPETTHEAAAIDEDVLRYQFVKKKGYVRLHTNKGDLNLELHCDLTPKTCENFIRLCKKHYYDGTIFHRSIRNFVIQGGDPTGTGTGGESYWGKPFKDEFRPNLSHTGRGILSMANSGPNSNRSQFFITFRSCAYLDKKHTIFGRVVGGFDVLTAMENVESDPKTDRPKEEIRIDATTVFVDPYEEADAQIAQERKTQLKVAPETKVKSSQPQAGSQGPQTFRQGVGKYINPAATKRAAEEEPSTSATVPMSKKKPSRGFGDFSSW

SEQ ID NO: 66 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 67 - protein, 318 aa, Homo sapiens
MAAAAAAAAEQQSSNGPVKKSMREKAVERRSVNKEHNSNFKAGYIPIDEDRLHKTGLRGRKGNLAICVIILLFILAVINLIITLVIWAVIRIGPNGCDSMEFHESGLLRFKQVSDMGVIHPLYKSTVGGRRNENLVITGNNQPIVFQQGTTKLSVENNKTSITSDIGMQFFDPRTQNILFSTDYETHEFHLPSGVKSLNVQKASTERITSNATSDLNIKVDGRAIVRGNEGVFIMGKTIEFHMGGNMELKAENSIILNGSVMVSTTRLPSSSSGDQLGSGDWVRYKLCMCADGTLFKVQVTSQNMGCQISDNPCGNTH

SEQ ID NO: 68 - protein, 572 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 69 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 70 - protein, 255 aa, Homo sapiens
MKALLALPLLLLLSTPPCAPQVSGIRGDALERFCLQQPLDCDDIYAQGYQSDGVYLIYPSGPSVPVPVFCDMTTEGGKWTVFQKRFNGSVSFFRGWNDYKLGFGRADGEYWLGLQNMHLLTLKQKYELRVDLEDFENNTAYAKYADFSISPNAVSAEEDGYTLFVAGFEDGGAGDSLSYHSGQKFSTFDRDQDLFVQNCAALSSGAFWFRSCHFANLNGFYLGGSHLSYANGINWAQWKGFYYSLKRTEMKIRRA

SEQ ID NO: 71 - protein, 1019 aa, Homo sapiens
MLQGTCSVLLLWGILGAIQAQQQEVISPDTTERNNNCPEKTDCPIHVYFVLDTSESVTMQSPTDILLFHMKQFVPQFISQLQNEFYLDQVALSWRYGGLHFSDQVEVFSPPGSDRASFIKNLQGISSFRRGTFTDCALANMTEQIRQDRSKGTVHFAVVITDGHVTGSPCGGIKLQAERAREEGIRLFAVAPNQNLKEQGLRDIASTPHELYRNDYATMLPDSTEIDQDTINRIIKVMKHEAYGECYKVSCLEIPGPSGPKGYRGQKGAKGNMGEPGEPGQKGRQGDPGIEGPIGFPGPKGVPGFKGEKGEFGADGRKGAPGLAGKNGTDGQKGKLGRIGPPGCKGDPGNRGPDGYPGEAGSPGERGDQGGKGDPGRPGRRGPPGEIGAKGSKGYQGNSGAPGSPGVKGAKGGPGPRGPKGEPGRRGDPGTKGSPGSDGPKGEKGDPGPEGPRGLAGEVGNKGAKGDRGLPGPRGPQGALGEPGKQGSRGDPGDAGPRGDSGQPGPKGDPGRPGFSYPGPRGAPGEKGEPGPRGPEGGRGDFGLKGEPGRKGEKGEPADPGPPGEPGPRGPRGVPGPEGEPGPPGDPGLTECDVMTYVRETCGCCDCEKRCGALDVVFVIDSSESIGYTNFTLEKNFVINVVNRLGAIAKDPKSETGTRVGVVQYSHEGTFEAIQLDDERIDSLSSFKEAVKNLEWIAGGTWTPSALKFAYDRLIKESRRQKTRVFAVVITDGRHDPRDDDLNLRALCDRDVTVTAIGIGDMFHEKHESENLYSIACDKPQQVRNMTLFSDLVAEKFIDDMEDVLCPDPQIVCPDLPCQTELSVAQCTQRPVDIVFLLDGSERLGEQNFHKARRFVEQVARRLTLARRDDDPLNARVALLQFGGPGEQQVAFPLSHNLTAIHEALETTQYLNSFSHVGAGVVHAINAIVRSPRGGARRHAELSFVFLTDGVTGNDSLHESAHSMRKQNVVPTVLALGSDVDMDVLTTLSLGDRAAVFHEKDYDSLAQPGFFDRFIRWIC

SEQ ID NO: 72 - protein, 640 aa, Homo sapiens
MAAGGTGGLREEQRYGLSCGRLGQDNITVLHVKLTETAIRALETYQSHKNLIPFRPSIQFQGLHGLVKIPKNDPLNEVHNFNFYLSNVGKDNPQGSFDCIQQTFSSSGASQLNCLGFIQDKITVCATNDSYQMTRERMTQAEEESRNRSTKVIKPGGPYVGKRVQIRKAPQAVSDTVPERKRSTPMNPANTIRKTHSSSTISQRPYRDRVIHLLALKAYKKPELLARLQKDGVNQKDKNSLGAILQQVANLNSKDLSYTLKDYVFKELQRDWPGYSEIDRRSLESVLSRKLNPSQNAAGTSRSESPVCSSRDAVSSPQKRLLDSEFIDPLMNKKARISHLTNRVPPTLNGHLNPTSEKSAAGLPLPPAAAAIPTPPPLPSTYLPISHPPQIVNSNSNSPSTPEGRGTQDLPVDSFSQNDSIYEDQQDKYTSRTSLETLPPGSVLLKCPKPMEENHSMSHKKSKKKSKKHKEKDQIKKHDIETIEEKEEDLKREEEIAKLNNSSPNSSGGVKEDCTASMEPSAIELPDYLIKYIAIVSYEQRQNYKDDFNAEYDEYRALHARMETVARRFIKLDAQRKRLSPGSKEYQNVHEEVLQEYQKIKQSSPNYHEEKYRCEYLHNKLAHIKRLIGEFDQQQAESWS

SEQ ID NO: 73 - protein, 495 aa, Homo sapiens
MKKRKELNALIGLAGDSRRKKPKKGPSSHRLLRTEPPDSDSESSSEEEEEFGVVGNRSRFAKGDYLRCCKICYPLCGFVILAACVVACVGLVWMQVALKEDLDALKEKFRTMESNQKSSFQEIPKLNEELLSKQKQLEKIESGEMGLNKVWINITEMNKQISLLTSAVNHLKANVKSAADLISLPTTVEGLQKSVASIGNTLNSVHLAVEALQKTVDEHKKTMELLQSDMNQHFLKETPGSNQIIPSPSATSELDNKTHSENLKQDILYLHNSLEEVNSALVGYQRQNDLKLEGMNETVSNLTQRVNLIESDVVAMSKVEKKANLSFSMMGDRSATLKRQSLDQVTNRTDTVKIQSIKKEDSSNSQVSKLREKLQLISALTNKPESNRPPETADEEQVESFTSKPSALPKFSQFLGDPVEKAAQLRPISLPGVSSTEDLQDLFRKTGQDVDGKLTYQEIWTSLGSAMPEPESLRAFDSDGDGRYSFLELRVALGI

SEQ ID NO: 74 - protein, 107 aa, Homo sapiens
MASGAARWLVLAPVRSGALRSGPSLRKDGDVSAAWSGSGRSLVPSRSVIVTRSGAILPKPVKMSFGLLRVFSIVIPFLYVGTLISKNFAALLEEHDIFVPEDDDDDD

SEQ ID NO: 75 - protein, 529 aa, Homo sapiens [sequence omitted]

SEQ ID NO: 76 - protein, 257 aa, Homo sapiens
MGTVRPPRPSLLLVSTRESCLFLLFCLHLGAACPQPCRCPDHAGAVAVFCSLRGLQEVPEDIPANTVLLKLDANKISHLPDGAFQHLHRLRELDLSHNAIEAIGSATFAGLAGGLRLLDLSYNRIQRIPKDALGKLSAKIRLSHNPLHCECALQEALWELKLDPDSVDEIACHTSVQEEFVGKPLVQALDAGASLCSVPHRTTDVAMLVTMFGWFAMVIAYVVYYVRHNQEDARRHLEYLKSLPSAPASKDPIGPGP

SEQ ID NO: 77 - protein, 327 aa, Homo sapiens
MAELPGPFLCGALLGFLCLSGLAVEVKVPTEPLSTPLGKTAELTCTYSTSVGDSFALEWSFVQPGKPISESHPILYFTNGHLYPTGSKSKRVSLLQNPPTVGVATLKLTDVHPSDTGTYLCQVNNPPDFYTNGLGLINLTVLVPPSNPLCSQSGQTSVGGSTALRCSSSEGAPKPVYNWVRLGTFPTPSPGSMVQDEVSGQLILTNLSLTSSGTYRCVATNQMGSASCELTLSVTEPSQGRVAGALIGVLLGVLLLSVAAFCLVRFQKERGKKPKETYGGSDLREDAIAPGISEHTCMRADSSKGFLERPSSASTVTTTKSKLPMVV

SEQ ID NO: 78 - protein, 94 aa, Homo sapiens
MAAFPHKIIFFLVCSTLTHVAFSGIFNKRDFTRWPKPRCKMYIPLDPDYNADCPNVTAPVCASNGHTFQNECFFCVEQREFHYRIKFEKYGKCD

SEQ ID NO: 79 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 80 - protein, 67 aa, Homo sapiens
MKLLLLTLTVLLLLSQLTPGGTQRCWNLYGKCRYRCSKKERVYVYCINNKMCCVKPKYQPKERWWPF

SEQ ID NO: 81 - protein, 254 aa, Homo sapiens
MPIKPVGWICGQVLKNFSGRIEGIQKAIMDLVDEFKDEFPTILRLSQSNQKREPAQKTSKIRMAIALAKINRATLIRGLNSISRSSKSVAKLLHPQLACRLLELRDISGRLLREVNAPRQPLYNIQVRKGSLFEIISFPAKTALTSIIYASYAALIYLAVCVNAVLKKVKNIFQEEESIRQNREESENCRKAFSEPVLSEPMFAEGEIKAKPYRSLPEKPDISDYPKLLANKQSNNIQVLHSVFDQSAEMNEQI

SEQ ID NO: 82 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 83 - protein, 430 aa, Homo sapiens
MAANLSRNGPALQEAYVRVVTEKSPTDWALFTYEGNSNDIRVAGTGEGGLEEMVEELNSGKVMYAFCRVKDPNSGLPKFVLINWTGEGVNDVRKGACASHVSTMASFLKGAHVTINARAEEDVEPECIMEKVAKASGANYSFHKESGRFQDVGPQAPVGSVYQKTNAVSEIKRVGKDSFWAKAEKEEENRRLEEKRRAEEAQRQLEQERRERELREAARREQRYQEQGGEASPQRTWEQQQEVVSRNRNEQESAVHPREIFKQKERAMSTTSISSPQPGKLRSPFLQKQLTQPETHFGREPAAAISRPRADLPAEEPAPSTPPCLVQAEEEAVYEEPPEQETFYEQPPLVQQQGAGSEHIDHHIQGQGLSGQGLCARALYDYQAADDTEISFDPENLITGIEVIDEGWWRGYGPDGHFGMFPANYVELIE

SEQ ID NO: 84 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 85 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 86 - protein, 407 aa, Homo sapiens
MDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 87 - protein, 163 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 88 - protein, 699 aa, Homo sapiens
MKQRFSALQLLKLLLLLQPPLPRALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPRLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPSHCCAFRNLPTKEQNFSHSISENFSKQCESTVRKVNNKTLYSSMLAESELSGWDYEYGFCLPKTPRCAPEPDAFNPCEDIMGYDFLRVLIWLINILAIMGNMTVLFVLLTSRYKLTVPRFLMCNLSFADFCMGLYLLLIASVDSQTKGQYYNHAIDWQTGSGCSTAGFFTVFASELSVYTLTVITLERWHTITYAIHLDQKLRLRHAILIMLGGWLFSSLIAMLPLVGVSNYMKVSICFPMDVETTLSQVYILTILILNVVAFFIICACYIKIYFAVRNPELMATNKDTKIAKKMAILIFTDFTCMAPISFFAISAAFKVPLITVTNSKVLLVLFYPINSCANPFLYAIFTKTFQRDFFLLLSKFGCCKRRAELYRRKDFSAYTSNCKNGFTGSNKPSQSTLKLSTLHCQGTALLDKTRYTEC

SEQ ID NO: 89 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 90 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 91 - protein, 343 aa, Homo sapiens
MWSHLNRLLFWSIFSSVTCRKAVLDCEAMKTNEFPSPCLDSKTKVVMKGQNVSMFCSHKNKSLQITYSLFRRKTHLGTQDGKGEPAIFNLSITEAHESGPYKCKAQVTSCSKYSRDFSFTIVDPVTSPVLNIMVIQTETDRHITLHCLSVNGSLPINYTFFENHVAISPAISKYDREPAEFNLTKKNPGEEEEYRCEAKNRLPNYATYSHPVTMPSTGGDSCPFCLKLLLPGLLLLLVVIILILAFWVLPKYKTRKAMRNNVPRDRGDTAMEVGIYANILEKQAKEESVPEVGSRPCVSTAQDEAKHSQELQYATPVFQEVAPREQEACDSYKSGYVYSELNF

(19) United States
(12) Patent Application Publication
(10) Pub. No.: US 2022/0260594 A1
(43) Pub. Date: Aug. 18, 2022

(54) METHODS FOR DIAGNOSING MULTIPLE SCLEROSIS

(71) Applicant: OXFORD UNIVERSITY INNOVATION LIMITED, Oxford (GB)

(72) Inventors: Fay Probert, Oxford (GB); Daniel Anthony, Oxford (GB)

(73) Assignee: OXFORD UNIVERSITY INNOVATION LIMITED, Oxford (GB)

(21) Appl. No.: 17/624,764

(22) PCT Filed: Jul. 6, 2020
(86) PCT No.: PCT/GB2020/051615

§ 371 Date: Jan. 4, 2022

(30) Foreign Application Priority Data
Jul. 4, 2019 (GB) 1909619.7

(51) Int. Cl. G01N 33/68

(52) U.S. Cl. CPC G01N 33/6896; G01N 2800/285

(57) ABSTRACT

The present invention relates to a method for determining conversion of a subject from clinically isolated syndrome (CIS) to clinically definite multiple sclerosis (CDMS), the method comprising: a. providing a sample obtained from the subject, wherein the subject has, or is suspected of having, CIS (preferably has CIS); b. measuring a concentration of: i. one or more polypeptides in the sample; and/or ii. one or more metabolites in the sample; c. comparing the measured concentration with the concentration of the same one or more polypeptides and/or metabolites, respectively, in a reference standard; and d. determining that the subject will convert from CIS to CDMS based on the comparison, or determining that the subject will not convert from CIS to CDMS based on the comparison. The invention also relates to methods for diagnosing multiple sclerosis (MS), to methods for determining prognosis of MS and to therapeutics and their uses in a method of treating MS in a subject.

The present invention relates to multiple sclerosis (MS) and methods for diagnosing the same.

Multiple sclerosis (MS) is the most common cause of progressive disability in the Western world. MS can be divided into several subtypes: Relapsing-Remitting MS (RRMS), Secondary-Progressive MS (SPMS), and Primary-Progressive MS (PPMS). RRMS is defined by discrete and temporary periods of disability worsening/disease flare-up (relapses) followed by recovery or periods with no disability worsening or disease activity (remission). RRMS is the most common type of MS, affecting ˜85% of MS patients. The majority of RRMS patients will eventually proceed to develop SPMS. SPMS diagnosis, by definition, must follow an RRMS diagnosis. This type of MS is characterised by continued accrual of disability and progressive worsening of symptoms over time, typically with no more discrete relapses. PPMS is the rarest type of MS, affecting ˜10% of patients. In PPMS, the patient never has a relapsing/remitting phase and enters the progressive phase from onset.

MS presents with an initial neurological attack, termed clinically isolated syndrome (CIS). Some patients who experience CIS will not convert to clinically definite MS (CDMS) (non-convertors), and, for those who do (convertors), time to conversion varies. The McDonald criteria is typically used to diagnose MS (e.g. RRMS) in the clinic and uses brain MRI in an attempt at early diagnosis (at the point of CIS) without the need for waiting for a second attack (indicative of true CDMS). The McDonald criteria is reviewed every few years, with the most recent criteria summarised in Lancet Neurol. 2018 February; 17(2):162-173. doi: 10.1016/S1474-4422(17)30470-2. Previous diagnostic criteria can be found at: 2010 Diagnostic Criteria: Ann Neurol. 2011 February; 69(2):292-302; and 2001 (original) diagnostic criteria: Ann Neurol. 2001 July; 50(1):121-7).

While the revised McDonald criteria aims to diagnose MS early using a combination of clinical and radiological data along with the presence of oligoclonal bands (OCB), it is associated with a number of disadvantages. First, 27% of CDMS patients do not fulfil McDonald MRI criteria at the point of CIS. Secondly, 50% of patients who do fulfil McDonald MRI criteria never have a second attack and never convert to CDMS (potentially incorrectly diagnosed/treated). Thirdly, there is no prognostic measure that is able to identify fast converters. Indeed, only 59% of OCB positive CIS patients convert to CDMS within 4 years.

There is thus a need for an improved method of diagnosing MS, and, in particular, an improved method for determining conversion of a subject from CIS to CDMS.

The present invention provides a solution to at least one of the problems described above.

The present inventors have surprisingly found that a method comprising measuring a concentration of one or more polypeptides described herein and/or one or more metabolites described herein in a sample from a subject allows for an improved method of diagnosing MS.

The methods of the invention allow for improved diagnosis of MS per se, CIS, and/or CDMS, as well as determining prognosis of MS. In particular, the methods of the invention allow determination of conversion of a subject from CIS to CDMS (e.g. within 4 years). Advantageously, the methods of the invention are particularly accurate and/or sensitive and/or specific.

Thus, in one aspect the invention provides a method for determining conversion of a subject from clinically isolated syndrome (CIS) to clinically definite multiple sclerosis (CDMS), the method comprising:
a. providing a sample obtained from the subject, wherein the subject has, or is suspected of having, CIS (preferably has CIS);
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard; and
d. determining that the subject will convert from CIS to CDMS based on the comparison, or determining that the subject will not convert from CIS to CDMS based on the comparison.

In one embodiment the invention provides a method for determining conversion of a subject from clinically isolated syndrome (CIS) to clinically definite multiple sclerosis (CDMS), the method comprising:
a. providing a sample obtained from the subject, wherein the subject has, or is suspected of having, CIS;
b. measuring a concentration of one or more polypeptides in the sample;
c. comparing the measured concentration with the concentration of the same one or more polypeptides in a reference standard; and
d. determining that the subject will convert from CIS to CDMS based on the comparison, or determining that the subject will not convert from CIS to CDMS based on the comparison.

In another embodiment the invention provides a method for determining conversion of a subject from clinically isolated syndrome (CIS) to clinically definite multiple sclerosis (CDMS), the method comprising:
a. providing a sample obtained from the subject, wherein the subject has, or is suspected of having, CIS;
b. measuring a concentration of one or more metabolites in the sample;
c. comparing the measured concentration with the concentration of the same one or more polypeptides in a reference standard; and
d. determining that the subject will convert from CIS to CDMS based on the comparison, or determining that the subject will not convert from CIS to CDMS based on the comparison.

“CIS” may refer to a first episode of neurologic symptoms that lasts for at least 24 hours and that is caused by inflammation and/or demyelination in the brain of the subject. Preferably, said symptom is one that is suggestive of MS with no other clinically-reasonable explanation. “CIS” is preferably associated with a brain lesion.

“CDMS” may refer to the stage at which at least one further episode of neurological symptoms have occurred in a subject. Preferably, CDMS is diagnosed when other possible diagnoses have been ruled out. “CDMS” may be associated with at least a further brain lesion when compared to CIS.

The methods of the invention preferably allow for a determination of the rate of conversion of a subject from CIS to CDMS. In one embodiment, a method of the invention determines whether or not a subject will convert from CIS to CDMS within a period of 10 years from CIS occurring. In another embodiment a method of the invention determines whether or not a subject will convert from CIS to CDMS within a period of 5 years from CIS occurring. Preferably, a method of the invention determines whether or not a subject will convert from CIS to CDMS within a period of 4 years from CIS occurring.

By using the methods of the invention, the convertor status of a subject can be determined. Advantageously, said status can be used to determine a suitable therapeutic strategy for the subject. For example, if the subject is identified as being a convertor to CDMS (e.g. a rapid convertor) early therapeutic intervention can be employed, which may, ultimately, delay conversion to CDMS. Thus, in one aspect the invention may comprise administering a suitable therapeutic to a subject: determined to be a convertor (e.g. a rapid convertor), diagnosed as having MS, and/or determined to have a poor prognosis in accordance with a method of the invention. In one embodiment the invention may comprise administering to the subject predicted to be a convertor a suitable therapeutic that delays conversion.

In one aspect the invention provides a therapeutic for use in a method of treating MS in a subject, said method comprising:
a. obtaining the results of a method of the invention; and
b. administering a therapeutic for MS when it has been determined that a subject will convert from CIS to CDMS.

In one embodiment the invention provides a therapeutic for use in a method of treating MS in a subject, said method comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. determining that the subject will convert from CIS to CDMS based on the comparison, or determining that the subject will not convert from CIS to CDMS based on the comparison; and
e. administering a therapeutic for MS when it has been determined that a subject will convert from CIS to CDMS.

In one aspect the invention provides a therapeutic for use in a method of treating MS in a subject, said method comprising:
a. obtaining the results of a method of the invention; and
b. administering a therapeutic for MS when a subject is diagnosed as having MS.

In one embodiment the invention provides a therapeutic for use in a method of treating MS in a subject, said method comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. diagnosing MS, or not diagnosing MS based on the comparison; and
e. administering a therapeutic for MS when a subject is diagnosed as having MS.

In one aspect the invention provides a therapeutic for use in a method of treating MS in a subject, said method comprising:
a. obtaining the results of a method of the invention; and
b. administering a therapeutic for MS when a subject is determined to have a poor prognosis.

In one embodiment the invention provides a therapeutic for use in a method of treating MS in a subject, said method comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. determining that the subject's prognosis is poor based on the comparison or determining that the subject's prognosis is good based on the comparison; and
e. administering a therapeutic for MS when a subject is determined to have a poor prognosis.

In one aspect the invention provides a method of treating MS in a subject, said method comprising:
a. obtaining the results of a method of the invention; and
b. administering a therapeutic for MS when it has been determined that a subject will convert from CIS to CDMS.

In one embodiment the invention provides a method of treating MS in a subject, said method comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. determining that the subject will convert from CIS to CDMS based on the comparison, or determining that the subject will not convert from CIS to CDMS based on the comparison; and
e. administering a therapeutic for MS when it has been determined that a subject will convert from CIS to CDMS.

In one aspect the invention provides a method of treating MS in a subject, said method comprising:
a. obtaining the results of a method of the invention; and
b. administering a therapeutic for MS when a subject is diagnosed as having MS.

In one embodiment the invention provides a method of treating MS in a subject, said method comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. diagnosing MS, or not diagnosing MS based on the comparison; and
e. administering a therapeutic for MS when a subject is diagnosed as having MS.

In one aspect the invention provides a method of treating MS in a subject, said method comprising:
a. obtaining the results of a method of the invention; and
b. administering a therapeutic for MS when a subject is determined to have a poor prognosis.

In one embodiment the invention provides a method of treating MS in a subject, said method comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. determining that the subject's prognosis is poor based on the comparison or determining that the subject's prognosis is good based on the comparison; and
e. administering a therapeutic for MS when a subject is determined to have a poor prognosis.

In one aspect, the invention provides use of a therapeutic in the manufacture of a medicament for treating MS, comprising:
a. obtaining the results of a method of the invention; and
b. administering a therapeutic for MS when it has been determined that a subject will convert from CIS to CDMS.

In one embodiment the invention provides use of a therapeutic in the manufacture of a medicament for treating MS, comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. determining that the subject will convert from CIS to CDMS based on the comparison, or determining that the subject will not convert from CIS to CDMS based on the comparison; and
e. administering a therapeutic for MS when it has been determined that a subject will convert from CIS to CDMS.

In one aspect the invention provides use of a therapeutic in the manufacture of a medicament for treating MS, comprising:
a. obtaining the results of a method of the invention; and
b. administering a therapeutic for MS when a subject is diagnosed as having MS.

In one embodiment the invention provides use of a therapeutic in the manufacture of a medicament for treating MS, comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. diagnosing MS, or not diagnosing MS based on the comparison; and
e. administering a therapeutic for MS when a subject is diagnosed as having MS.

In one aspect the invention provides use of a therapeutic in the manufacture of a medicament for treating MS, comprising:
a. obtaining the results of a method of the invention; and
b. administering a therapeutic for MS when a subject is determined to have a poor prognosis.

In one embodiment the invention provides use of a therapeutic in the manufacture of a medicament for treating MS, comprising:
a. providing a sample obtained from the subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard;
d. determining that the subject's prognosis is poor based on the comparison or determining that the subject's prognosis is good based on the comparison; and
e. administering a therapeutic for MS when a subject is determined to have a poor prognosis.

A “rapid convertor” (used synonymously with “fast convertor” herein) may be a subject who converts from CIS to CDMS within 10 years of CIS occurring. In one embodiment a “rapid convertor” is a subject who converts from CIS to CDMS within 5 years of CIS occurring. Preferably, a “rapid convertor” is a subject who converts from CIS to CDMS within 4 years of CIS occurring.

A “slow convertor” may be a subject who converts from CIS to CDMS in more than 10 years of CIS occurring. In one embodiment a “slow convertor” is a subject who converts from CIS to CDMS in more than 5 years of CIS occurring. Preferably, a “slow convertor” is a subject who converts from CIS to CDMS in more than 4 years of CIS occurring.

The methods of the invention may allow the determination of whether a subject is a “rapid convertor” or a “slow convertor”. In one embodiment, where a method is a method for determining conversion of a subject from CIS to CDMS and where it has been determined that the subject will convert from CIS to CDMS, preferably this means that the subject is a fast convertor. In contrast, in one embodiment, where a method is a method for determining conversion of a subject from CIS to CDMS and where it has been determined that the subject will not convert from CIS to CDMS, preferably this means that the subject is a slow convertor or a non-convertor.

In one aspect the invention provides a method for diagnosing multiple sclerosis (MS), the method comprising:
a. providing a sample obtained from a subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard; and
d. diagnosing MS, or not diagnosing MS based on the comparison.

In one aspect there is provided a method for diagnosing Multiple Sclerosis (MS) in a (preferably human) test subject, the method comprising:
(i) determining the concentration of:
   one or more polypeptides in a sample from the subject; and/or
   one or more metabolites in a sample from the subject;
(ii) comparing the concentrations of said one or more polypeptides and/or metabolites in the sample with the concentrations of the same polypeptides and/or metabolites in at least one reference standard; and
(iii) identifying a concentration difference for said one or more polypeptides and/or metabolites in the sample relative to the reference standard;
   wherein said concentration differences correlate with the presence of MS.

In one aspect the invention provides a method for diagnosing relapsing-remitting multiple sclerosis (RRMS), the method comprising:
a. providing a sample obtained from a subject;
b. measuring a concentration of:
   i. one or more polypeptides in the sample; and/or
   ii. one or more metabolites in the sample;
c. comparing the measured concentration with the concentration of the same one or more polypeptides and/or metabolites, respectively, in a reference standard; and
d. diagnosing RRMS, or not diagnosing RRMS based on the comparison.

In a related aspect the invention provides a method for determining prognosis of multiple sclerosis (MS), the method comprising:
a. providing a sample obtained from a subject;
b. measuring a concentration of:
   i. one or more polypeptides in the sample; and/or
   ii. one or more metabolites in the sample;
c. comparing the concentration with the concentration of the same one or more polypeptides and/or metabolites, respectively, in a reference standard; and
d. determining that the subject's prognosis is poor based on the comparison or determining that the subject's prognosis is good based on the comparison.

In one aspect the invention provides a method, the method comprising:
a. obtaining a biofluid sample derived from a subject having, or suspected of having, CIS;
b. assaying the biofluid sample for a concentration of:
   i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)₃/free choline, formate, 3-hydroxybutyrate, myo-inositol, NAC1/=CH—CH2-CH2-, glucose, glutamine, and lactate.

In one aspect the invention provides a method for diagnosing multiple sclerosis (MS), the method comprising:
a. providing a sample obtained from a subject;
b. measuring a concentration of:
   i. one or more polypeptides in the sample, wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
   ii. one or more metabolites in the sample, wherein the one or more metabolites are selected from: creatinine, creatine, mobile lipoprotein (—CH2-)n resonances (VLDL and LDL), isoleucine, leucine, mobile lipoprotein —CH3 resonances (HDL and LDL), betaine, mobile —N(CH3)₃/free choline, formate, 3-hydroxybutyrate, myo-inositol, NAC1/=CH—CH2-CH2-, glucose, glutamine, and lactate;
c. comparing the measured concentration with the concentration of the same one or more polypeptides and/or metabolites, respectively, in a reference standard; and
d. diagnosing MS, or not diagnosing MS based on the comparison.

In a related aspect the invention provides a method for determining prognosis of multiple sclerosis (MS), the method comprising:
a. providing a sample obtained from a subject;
b. measuring a concentration of:
   i. one or more polypeptides in the sample, wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
   ii. one or more metabolites in the sample, wherein the one or more metabolites are selected from: creatinine, creatine, mobile lipoprotein (—CH2-)n resonances (VLDL and LDL), isoleucine, leucine, mobile lipoprotein —CH3 resonances (HDL and LDL), betaine, mobile —N(CH3)₃/free choline, formate, 3-hydroxybutyrate, myo-inositol, NAC1/=CH—CH2-CH2-, glucose, glutamine, and lactate;
c. comparing the concentration with the concentration of the same one or more polypeptides and/or metabolites, respectively, in a reference standard; and
d. determining that the subject's prognosis is poor based on the comparison or determining that the subject's prognosis is good based on the comparison.

In one aspect the invention provides a method for predicting whether a subject will convert from clinically isolated syndrome (CIS) to clinically definite multiple sclerosis (CDMS), the method comprising:
a. obtaining a biofluid sample derived from a subject having, or suspected of having, CIS;
b. assaying the biofluid sample for a concentration of:
   i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
   ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the assayed concentration with the concentration of the same one or more polypeptides and/or metabolites, respectively, in a reference standard; and
d. determining that the subject will convert from CIS to CDMS based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is increased when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
determining that the subject will not convert from CIS to CDMS based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is decreased or the same when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is decreased when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard, thereby predicting whether a subject will convert from CIS to CDMS.

In one aspect the invention provides a method for diagnosing MS, the method comprising:
a. obtaining a biofluid sample derived from a subject;
b. assaying the biofluid sample for a concentration of:
   i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
   ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)₃/free choline, formate, 3-hydroxybutyrate, myo-inositol, NAC1/=CH—CH2-CH2-, glucose, glutamine, and lactate;
c. comparing the assayed concentration with the concentration of the same one or more polypeptides and/or metabolites, respectively, in a reference standard; and
d. diagnosing MS based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is increased when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is increased or the same when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
not diagnosing MS when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is decreased or the same when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is decreased when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard, thereby diagnosing or not diagnosing MS.

In one aspect, the invention provides a method for predicting prognosis of MS, the method comprising:
a. obtaining a biofluid sample derived from a subject;
b. assaying the biofluid sample for a concentration of:
   i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
   ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)₃/free choline, formate, 3-hydroxybutyrate, myo-inositol, NAC1/=CH—CH2-CH2-, glucose, glutamine, and lactate;
c. comparing the assayed concentration with the concentration of the same one or more polypeptides and/or metabolites, respectively, in a reference standard; and
d. determining that the subject's prognosis is poor based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is increased when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is increased or the same when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
determining that the subject's prognosis is good based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is decreased or the same when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is decreased when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard, thereby predicting the prognosis of MS.

In one embodiment the invention provides a method for diagnosing multiple sclerosis (MS), the method comprising:
a. providing a sample obtained from a subject;
b. measuring a concentration of one or more polypeptides in the sample,
   wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1;
c. comparing the measured concentration with the concentration of the same one or more polypeptides in a reference standard; and
d. diagnosing MS, or not diagnosing MS based on the comparison.

In one embodiment the invention provides a method for diagnosing multiple sclerosis (MS), the method comprising:
a. providing a sample obtained from a subject;
b. measuring a concentration of one or more metabolites in the sample, wherein the one or more metabolites are selected from: creatinine, creatine, mobile lipoprotein (—CH2-) n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the measured concentration with the concentration of the same one or more metabolites in a reference standard; and
d. diagnosing MS, or not diagnosing MS based on the comparison.

In one embodiment the invention provides a method, the method comprising:
a. obtaining a biofluid sample derived from a subject having, or suspected of having, CIS;
b. assaying the biofluid sample for a concentration of:
   i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
   ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)₃/free choline, formate, 3-hydroxybutyrate, myo-inositol, NAC1/=CH—CH2-CH2-, glucose, glutamine, and lactate.
c. comparing the assayed concentration with the concentration of the same one or more polypeptides and/or metabolites, respectively, in a reference standard; and
d. determining that the subject will convert from CIS to CDMS based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)₃/free choline, formate, glucose (CSF), NAC1/=CH—CH2-CH2-, and lactate is increased when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
determining that the subject will not convert from CIS to CDMS based on the comparison when:
   i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
   ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
   iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
   iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard.

In a related aspect the invention provides a method for determining prognosis of multiple sclerosis (MS), the method comprising:
a. providing a sample obtained from a subject;
b. measuring a concentration of one or more polypeptides in the sample, wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1;
c. comparing the concentration with the concentration of the same one or more polypeptides in a reference standard; and
d. determining that the subject's prognosis is poor based on the comparison or determining that the subject's prognosis is good based on the comparison.

In a related aspect the invention provides a method for determining prognosis of multiple sclerosis (MS), the method comprising:
a. providing a sample obtained from a subject;
b. measuring a concentration of one or more metabolites in the sample, wherein the one or more metabolites are selected from: creatinine, creatine, mobile lipoprotein (—CH2-) n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the concentration with the concentration of the same one or more metabolites in a reference standard; and
d. determining that the subject's prognosis is poor based on the comparison or determining that the subject's prognosis is good based on the comparison.

Preferably, a method of the invention allows for the diagnosis of MS, such as RRMS and/or CDMS.

The present invention may comprise detecting one or more polypeptides described herein (e.g. in Table 1). Thus, in one embodiment a polypeptide is one or more selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1.

Representative sequences for the polypeptides for use in the invention are described in the Sequence Listing herein, together with the appropriate UniProt Accession numbers. A polypeptide for use in the invention may be one or more shown as SEQ ID NOs: 1-91 or a variant thereof, such as a transcript isoform therefore. A polypeptide for use in a method of the invention may comprise (or consist of) a polypeptide sequence having at least 20%, 30%, 40%, 50%, 60%, 70%, 80%, 90%, 95%, 99% or 100% sequence identity to any one of SEQ ID NOs: 1-91. Thus, in one embodiment, the invention comprises measuring the concentration of one or more polypeptides having at least 20% sequence identity to any one of SEQ ID NOs: 1-91. In one embodiment, the invention comprises measuring the concentration of one or more polypeptides having at least 30%, 40%, 50%, 60%, 70%, 80%, 90%, 95%, 99% or 100% sequence identity to any one of SEQ ID NOs: 1-91. In one embodiment, the invention comprises measuring the concentration of one or more polypeptides having at least 70% (preferably at least 80%, 90%, or 95%, more preferably 100%) sequence identity to any one of SEQ ID NOs: 1-91.

In one embodiment a polypeptide is one or more selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, and Myeloid zinc finger 1.

Preferably, at least one polypeptide is selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, and RING finger protein 165.

More preferably, at least one of the polypeptides is selected from: Ribosomal protein S6 kinase alpha-5, DNA repair protein XRCC1, Cytosolic acyl coenzyme A thioester hydrolase, Cytoplasmic tyrosine-protein kinase BMX, and Cathepsin K.

Measuring a concentration of a polypeptide of the invention may be carried out by any means known to the person skilled in the art. For example, the polypeptide concentration can be measured directly (by measuring the amount/concentration of polypeptide itself) or indirectly by assessing gene expression, e.g. at the level of transcription. In one embodiment, mRNA of a target gene can be detected and quantified by e.g. Northern blotting or by quantitative reverse transcription PCR (RT-PCR). In one embodiment, gene expression levels are determined by measuring the mRNA/cDNA levels of the genes of the present invention, such as RNA sequencing (RNA-Seq).

In some embodiments the invention may employ high-throughput techniques. High-throughput techniques can be used to analyse whole genomes, proteomes and transcriptomes rapidly, providing data, including the expression levels, of all of the genes, polypeptides and transcripts in a sample. For example, RNA sequencing (RNA-Seq) may be used. The invention may comprise the use of transcriptomics. Typically, proteomics is carried out by mass-spectrometry, including tandem mass-spectrometry, and gel-based techniques, including differential in-gel electrophoresis.

Preferably, polypeptide concentrations are determined directly by analysing polypeptide amounts in a sample. Suitable techniques may include mass spectrometry, e.g. liquid chromatography and mass spectrometry (LC-MS/MS), enzyme-linked immunosorbent assay (ELISA) or a Luminex assay (commercially available from R&D Systems, USA). More preferably, a polypeptide concentration may be determined using a SOMAscan Assay (SomaLogic, Inc., Boulder, Colo., USA). Directly determining polypeptide concentrations by analysing polypeptide amounts in a sample has advantages over other non-direct techniques, such as nucleic acid-based techniques, e.g. transcriptomics/gene expression analysis. Specifically, directly determining polypeptide concentrations may be more accurate and/or sensitive and/or specific when compared to indirect techniques. For example, nucleic acid-based techniques may not directly correlate to the final polypeptide concentrations. Thus, in one embodiment methods of the invention do not use indirect techniques for determining polypeptide concentrations, for example, methods of the invention may not use nucleic acid-based techniques for determining polypeptide concentrations, such as RNA analysis and/or transcriptomics.

When compared to a non-convertor reference standard the concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein may be increased. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a non-convertor reference standard the concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein may be decreased or the same. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a non-convertor reference standard the concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 may be decreased. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a non-convertor reference standard the concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 may be increased or the same. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a convertor reference standard the concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein may be increased or the same. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a convertor reference standard the concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein may be decreased. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a convertor reference standard the concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 may be decreased or the same. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a convertor reference standard the concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 may be increased. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

The present invention may comprise detecting one or more metabolites described herein (e.g. in Table 2 or 3). Relevant NMR resonance values in for said metabolites are provided in Tables 2 and 3. Thus, in one embodiment a metabolite may be one or more selected from: creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate.

In some embodiments the metabolites may be one or more cerebrospinal fluid metabolites selected from: creatinine, creatine, isoleucine, leucine, betaine, formate, myo-inositol, glucose, glutamine, and lactate.

In another embodiment the metabolites may be one or more serum metabolites selected from: mobile lipoprotein (—CH2-)n resonances (VLDL and LDL), glucose, mobile lipoprotein —CH3 resonances (HDL and LDL), mobile —N(CH3)3/free choline, 3-hydroxybutyrate, and NAC1/=CH—CH2-CH2-.

When compared to a non-convertor reference standard the concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate may be increased. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a non-convertor reference standard the concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate may be decreased or the same. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a non-convertor reference standard the concentration of one or more metabolites selected from: creatinine, creatine, isoleucine, leucine, betaine, 3-hydroxybutyrate, myo-inositol, glucose (serum), and glutamine may be decreased. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a non-convertor reference standard the concentration of one or more metabolites selected from: creatinine, creatine, isoleucine, leucine, betaine, 3-hydroxybutyrate, myo-inositol, glucose (serum), and glutamine may be increased or the same. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a convertor reference standard the concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate may be increased or the same. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a convertor reference standard the concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate may be decreased. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a convertor reference standard the concentration of one or more metabolites selected from: creatinine, creatine, isoleucine, leucine, betaine, 3-hydroxybutyrate, myo-inositol, glucose (serum), and glutamine may be decreased or the same. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a convertor reference standard the concentration of one or more metabolites selected from: creatinine, creatine, isoleucine, leucine, betaine, 3-hydroxybutyrate, myo-inositol, glucose (serum), and glutamine may be increased. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

Preferably, one or more metabolites and one or more polypeptides may be used in a method of the invention.

The concentrations of the metabolites in a sample can be measured using any suitable technique known in the art. By way of example, the following techniques may be used to detect and quantify small molecules in solution, and are thus suitable for determining metabolite concentrations: Nuclear Magnetic Resonance (NMR) spectroscopy, mass spectrometry, gas chromatography, ultraviolet (UV) spectrometry (for example in combination with high-performance liquid chromatography [HPLC] as HPLC-UV), and infrared spectroscopy. A metabolite is preferably identified using NMR, more preferably ¹H-NMR.

In one embodiment, the concentration of one or more metabolites is determined using NMR spectroscopy. In one embodiment, the concentration of one or more metabolites is determined using mass spectrometry. In one embodiment, the concentration of one or more metabolites is determined using HPLC-UV. In one embodiment, the concentration of one or more metabolites is determined using infrared spectroscopy.

The concentration of a polypeptide and/or metabolite in a sample can be expressed in a number of different ways, for example as a molar concentration (number of moles of polypeptide/metabolite per unit volume of sample) or a mass concentration (mass of polypeptide/metabolite per unit volume of sample). Alternatively, the concentration of a polypeptide/metabolite can be expressed as parts per million (ppm) or parts per billion (ppb). Such ways of expressing the concentration of a small molecule in solution are known in the art. In some embodiments a concentration of a polypeptide and/or metabolite may be expressed relative to a standard or to another polypeptide and/or metabolite within the sample. For example, when techniques such as NMR are employed a concentration may be expressed as a relative spectral intensity.

Thus, in one embodiment, the concentration of a polypeptide/metabolite in a sample is the molar concentration of said polypeptide/metabolite. In one embodiment, the concentration of a polypeptide/metabolite in a sample is the mass concentration of said polypeptide/metabolite.

The concentration of a polypeptide/metabolite in a sample may be expressed in absolute terms, for example as absolute molar concentration or absolute mass concentration. Alternatively, the concentration of a polypeptide/metabolite in a sample can be expressed by comparison to the concentration of a different polypeptide/metabolite in the same sample (i.e. in relative terms). By way of example, the concentration of a polypeptide/metabolite in the sample can be normalised by comparison to the concentration of a different reference polypeptide/metabolite within the same sample.

The methods described herein are particularly sensitive and allow for accurate and/or sensitive and/or specific determination, diagnosis and/or prognosis when using only one polypeptide. Notably, even where the concentration of a polypeptide has not been found to be statistically-significantly changed when compared to a reference standard, said polypeptide has utility in a method of the invention, especially where used in combination with a further polypeptide and/or metabolite and/or when compared to multiple reference standards.

In some embodiments more than one polypeptide may be employed. In a preferred embodiment at least 5 polypeptides are employed in a method described herein.

The term “one or more” when used in the context of a polypeptide described herein may mean at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or 91 of the polypeptides. In one embodiment the term “one or more” when used in the context of a polypeptide described herein may mean at least 50 of the polypeptides. When carrying out a method herein, it is preferred that those polypeptides that are highest ranked in Table 1 are selected, e.g. where 5 polypeptides are employed, it is preferred that these are the 5 highest ranking polypeptides.

Similarly, the methods described herein are particularly sensitive and allow for accurate and/or sensitive and/or specific determination, diagnosis and/or prognosis when using only one metabolite. Notably, even where the concentration of a metabolite has not been found to be statistically-significantly changed when compared to a reference standard, said polypeptide has utility in a method of the invention, especially where used in combination with a further polypeptide and/or metabolite and/or when compared to multiple reference standards.

In some embodiments more than one metabolite may be employed. In a preferred embodiment at least 2 metabolites are employed in a method described herein.

The term “one or more” when used in the context of a metabolite described herein may mean at least 2, 3, 4, 5, 6, 7, 8, 9, 10, 11, 12, 13, 14 or 15 metabolites. When carrying out a method herein, it is preferred that those metabolites that are highest ranked in Table 2 or 3 are selected, e.g. where 2 metabolites are employed, it is preferred that these are the 2 highest ranking metabolites. For example at least one metabolite employed in a method of the invention may be creatinine, creatine, mobile lipoprotein (—CH2-)n resonances (VLDL and LDL), isoleucine or glucose (serum).

Preferably, one or more polypeptides and one or more metabolites may be used in a method of the invention. Advantageously, this allows for improved accuracy/sensitivity and/or specificity when compared to the use of one or more polypeptide or one or more metabolite only.

In one embodiment, a method of the invention has an accuracy of at least 65%, 70%, 71%, 72%, 73%, 74%, or 75%. Preferably, a method of the invention has an accuracy of at least 80 or 85%, such as at least 90%.

In some embodiments a method of the invention may further comprise determining a subject's oligoclonal band status (i.e. positive or negative). Additionally or alternatively, a method of the invention may further comprise measuring in a sample obtained from a subject: leukocyte concentration, mononuclear cell concentration (e.g. peripheral blood mononuclear cell [PBMC] concentrations), polynuclear cell concentration (e.g. polynuclear neutrophil concentrations), serum albumin ratio (e.g. CSF/serum albumin ratios), and total protein concentration (e.g. CSF total protein concentration).

When compared to a non-convertor reference standard the concentration of leukocytes, mononuclear cells and/or polynuclear cells may be increased. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a non-convertor reference standard the concentration of leukocytes, mononuclear cells and/or polynuclear cells may be decreased or the same. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a non-convertor reference standard the CSF/serum albumin ratio and/or total protein (e.g. CSF total protein) may be decreased. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a non-convertor reference standard the CSF/serum albumin ratio and/or total protein (e.g. CSF total protein) may be increased or the same. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a convertor reference standard the concentration of leukocytes, mononuclear cells and/or polynuclear cells may be increased or the same. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a convertor reference standard the concentration of leukocytes, mononuclear cells and/or polynuclear cells may be decreased. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

When compared to a convertor reference standard the CSF/serum albumin ratio and/or total protein (e.g. CSF total protein) may be decreased or the same. In one embodiment, in such cases: it is determined that a subject will convert from CIS to CDMS (preferably, it is determined that a subject will be a rapid convertor to CDMS); and/or a subject is diagnosed with MS (e.g. CDMS and/or RRMS); and/or it is determined that a subject's prognosis is poor.

When compared to a convertor reference standard the CSF/serum albumin ratio and/or total protein (e.g. CSF total protein) may be increased. In one embodiment, in such cases: it is determined that a subject will not convert from CIS to CDMS (or that a subject will be a slow convertor to CDMS); and/or a subject is not diagnosed with MS (e.g. CDMS and/or RRMS); and/or it is determined that a subject's prognosis is good.

The terms “subject” and “patient” are used synonymously herein. The “subject” may be a mammal, and preferably the subject is a human subject.

The sample that is to be tested using the method of the invention can be derived from any suitable biofluid. In one embodiment the biofluid is selected from cerebrospinal fluid (CSF), blood or urine that has been obtained from a subject. Preferably the sample is a CSF sample. Without wishing to be bound by theory, it is believed that any biofluid is suitable for use in the present invention, especially when the method employs the use of one or more metabolites. This has been evidenced by the fact that the majority of polypeptides identified in CSF herein are also present in serum (these biofluid compartments are ‘linked’, as with blood and urine polypeptides/metabolites can pass between the biofluids) and thus it is believed that the same detection methods could also be used in blood, including with the use of alternative blood fractions, such as plasma.

The term blood comprises whole blood, blood serum (henceforth “serum”) and blood plasma (henceforth “plasma”), preferably serum. Serum and plasma are derived from blood and thus may be considered as specific subtypes within the broader genus “blood”. Processes for obtaining serum or plasma from blood are known in the art. For example, it is known in the art that blood can be subjected to centrifugation in order to separate red blood cells, white blood cells, and plasma. Serum is defined as plasma that lacks clotting factors. Serum can be obtained by centrifugation of blood in which the clotting process has been triggered. Optionally, this can be carried out in specialised centrifuge tubes designed for this purpose.

A sample for use in a method of the present invention can be derived from a biofluid that has undergone processing after being obtained from a test subject. Alternatively, a sample can be derived from a biofluid that has not undergone any processing after being obtained from a test subject.

The methods of the invention thus encompass the use of samples that have undergone minimal or zero processing before testing. This provides a significant advantage over prior art methods in terms of time, cost and practicality. By way of example, a CSF sample obtained from a test subject may be tested directly using the method of the present invention, without further processing. Serum and plasma samples can be readily obtained from blood samples using simple and readily available techniques that are well known in the art, as described above.

In a preferred embodiment, the samples for use in a method of the invention are cell-free biofluid samples. In other words, the biofluid sample of the invention may be processed to remove cells. The term “cell-free biofluid samples” are biofluid samples that contain substantially no cells. The term “substantially no” when used in the context of cells herein may mean less than 10,000, 5,000, 1,000, 100 or 10 cells/ml. The term “substantially no” when used in the context of cells herein preferably means less than 1,000 cells/ml, more preferably no cells. In some embodiments the term “substantially no” when used in the context of cells herein may be expressed in absolute amounts. For example, the term “substantially no” when used in the context of cells herein may mean less than 10,000, 5,000, 1,000, 100 or 10 cells. Preferably less than 1,000 cells, more preferably no cells.

At least one advantage associated with the use of cell-free biofluid samples is that the measurement of polypeptides and/or metabolites is not adversely influenced by the populations of different cell types that may be present in an equivalent biofluid sample containing cells. Said populations of different cell types may have different polypeptide expression profiles. Moreover, the cell-free biofluid may not need to be (and is preferably not) subjected to any enrichment steps. Thus, in one embodiment, the cell-free biofluid sample is not enriched for white blood cells, preferably is not enriched for peripheral blood mononuclear cells (PBMCs), T-cells and/or monocytes. Preferably, the cell-free biofluid sample comprises substantially no white blood cells, e.g. substantially no PBMCs, T-cells and/or monocytes.

The methods of the invention comprise comparing a concentration of a polypeptide and/or metabolite to a reference standard. In one embodiment, a reference standard comprises (or consists of) a sample (e.g. a biofluid sample described herein) obtained from a reference subject or subjects, wherein the reference subject is a subject other than the subject being tested in a method of the invention.

In one embodiment, a “reference standard” comprises (or consists of) a set of data relating to the concentration of one or more polypeptides and/or metabolites in a sample obtained from a reference subject or subjects, wherein the reference subject is a subject other than the subject being tested in a method of the invention. The set of data may be derived by measuring the concentration of said one or more polypeptides and/or metabolites. Said measuring may be carried out using any suitable technique described herein.

In one embodiment, the reference standard comprises (or consists of) a set of data relating to the concentration of said one or more polypeptides and/or metabolites in a sample or samples derived from a single reference subject. In other embodiments, the reference standard comprises (or consists of) a set of data relating to the concentration of said one or more polypeptides and/or metabolites in a sample or samples derived from a plurality of reference subjects (e.g. two or more reference subjects). Thus, in one embodiment, the reference standard is derived by pooling data obtained from two or more (e.g. three, four, five, 10, 15, 20 or 25) reference subjects and calculating an average (for example, mean or median) concentration for each polypeptide and/or metabolite. Thus, the reference standard may reflect average concentrations of said one or more polypeptides and/or metabolites in a sample in a given population of reference subjects. Said concentrations may be expressed in absolute or relative terms, in the same manner as described above in relation to the sample that is to be tested using the method of the invention.

In one embodiment a method of the invention comprises the use of a plurality of reference standards. In such embodiments a method may comprise the use of a non-convertor (preferably CIS) reference standard and a convertor (preferably CDMS) reference standard.

In some embodiments a reference standard may be constructed based on polypeptide and/or metabolite concentrations for a known convertor and/or non-convertor population. In some embodiments the methods of the present invention comprise comparing measured concentrations of polypeptides and/or metabolites to the concentration of said polypeptides and/or metabolites (respectively) in both a convertor and a non-convertor reference standard (or a plurality of convertor and non-convertor reference standards) and determining to which reference standard the sample is most similar (thus allowing a determination/diagnosis according to a method of the invention).

A polypeptide and/or metabolite concentration in a reference standard may have been obtained (e.g. quantified) previously to a method of the invention.

When comparing concentrations between the sample and the reference standard, the way in which the concentrations are expressed is matched between the sample and the reference standard. Thus, an absolute concentration can be compared with an absolute concentration, and a relative concentration can be compared with a relative concentration.

A reference standard employed in the present invention may be of known convertor status.

In one embodiment the reference standard is a non-convertor reference standard. The term “non-convertor reference standard” as used herein encompasses a reference standard from a subject that has CIS or a reference standard from a healthy subject who does not have CIS, preferably a subject that has CIS. In one embodiment the term “non-convertor reference standard” as used herein refers to a reference standard from a subject that has CIS but who is a slow convertor (preferably who does not convert to CDMS by 10 or 5 years, more preferably 4 years from CIS). In a particularly preferred embodiment, a reference standard is a non-convertor reference standard from a subject that has CIS (e.g. a subject that has been diagnosed with CIS) and that does not convert to CDMS. In one embodiment a reference standard is not a reference standard from a healthy subject.

In another embodiment the reference standard is a convertor reference standard. The term “convertor reference standard” as used herein encompasses a reference standard from a subject that has CDMS or a reference standard from subject who has CIS and subsequently converted to CDMS by 10 or 5 years, more preferably by 4 years from CIS. In other words, the reference standard may have been obtained from a rapid convertor, when said rapid converter had CIS (pre-conversion). Preferably, a “convertor reference standard” is from a subject that has CDMS (e.g. a subject that has been diagnosed with CDMS).

The reference standard is typically derived from the same sample type (e.g. biofluid) as the sample that is being tested, thus allowing for an appropriate comparison between the two or more.

The methods of the present invention are in vitro methods. Thus, the methods can be carried out in vitro on an isolated sample that has been obtained from a subject.

The methods of the invention comprise comparing the measured concentrations of one or more polypeptides and/or metabolites to make a determination or diagnosis. Thus, said measured concentrations may correlate with a convertor status of a subject and/or with the presence of MS and/or with a poor prognosis. Said determination or diagnosis is typically based on measuring a concentration difference. The term “concentration difference” embraces both positive and negative differences. Thus, a concentration difference can mean that the concentration of a polypeptide and/or metabolite is higher in the sample being tested than in the reference standard. Alternatively, a concentration difference can mean that the concentration of a polypeptide and/or metabolite is lower in the sample than in the reference standard.

The comparison and/or identification of the presence or absence of a concentration difference (as described above) can be achieved using methods of statistical analysis. In one embodiment a method of statistical analysis suitable for use in the present invention includes orthogonal partial least squares discriminate analysis (OPLS-DA).

In one embodiment, the method of the invention further comprises recording the output of at least one step on a data-storage medium. By way of example, the method of the present invention can generate data relating to the subject, such data being recordable on a data-storage medium (for example, a form of computer memory such as a hard disk, compact disc, floppy disk, or solid state drive). Such data can comprise (or consist of) data relating to the concentration in a sample (from said subject) of any of one or more polypeptides and/or metabolites (as described) above.

In one aspect the invention provides a data-storage medium, comprising data obtained by a method according to the present invention.

In another aspect, the invention provides a device for use in a method of the invention, wherein said device is capable of performing the step of identifying: a concentration difference of one or more polypeptides and/or one or more metabolites in the sample when compared to the reference standard.

Treatment of multiple sclerosis (e.g. CIS and/or CDMS) may be carried out using any MS therapeutic known in the art. For example, therapy may be carried out by administering a disease modifying therapy, via cell-based treatments or via physiotherapy. A disease modifying therapy may be one or more selected from: alemtuzumab (e.g. Lemtrada), beta interferons (e.g. Avonex, Betaferon, Extavia, Plegridy, and/or Rebif), cladribine (e.g. Mavenclad), dimethyl fumarate (Tecfidera), fingolimod (Gilenya), glatiramer acetate (e.g. Copaxone and/or Brabio), natalizumab (e.g. Tysabri), ocrelizumab (e.g. Ocrevus), and teriflunomide (e.g. Aubagio). A cell-based treatment may comprise treatment with a hematopoietic cell or a functional equivalent, such as a hematopoietic stem cell and/or progenitor cell. For example, a cell-based treatment may be combined with chemotherapy (e.g. to ablate a subject's native stem cell and progenitor cell population) prior to transplanting the hematopoietic cell or functional equivalent. In some embodiments, the cells transplanted may be those of the subject removed prior to chemotherapy and treated such that, when transplanted, they will not contribute to (e.g. cause) MS and/or a symptom thereof.

The term “disorder” as used herein also encompasses a “disease”. In one embodiment the disorder is a disease. The disorder treated in accordance with the invention is MS.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment.

The term “treat” or “treating” as used herein refers to the disorder and/or a symptom thereof.

Therefore a therapeutic may be administered to a subject in a therapeutically effective amount or a prophylactically effective amount.

A “therapeutically effective amount” is any amount of a therapeutic formulation, which when administered alone or in combination to a subject for treating said disorder (or a symptom thereof) is sufficient to effect such treatment of the disorder, or symptom thereof.

A “prophylactically effective amount” is any amount of a therapeutic formulation that, when administered alone or in combination to a subject inhibits or delays the onset or reoccurrence of a disorder (or a symptom thereof). In some embodiments, the prophylactically effective amount prevents the onset or reoccurrence of a disorder entirely. “Inhibiting” the onset means either lessening the likelihood of a disorder's onset (or symptom thereof), or preventing the onset entirely.

Administration may be by any route known in the art and will typically be dependent on the nature of the therapeutic to be administered. For example, a therapeutic may be administered orally or parenterally. Methods of parenteral delivery include topical, intra-arterial, intramuscular, subcutaneous, intramedullary, intrathecal, intra-ventricular, intravenous, intraperitoneal, or intranasal administration.

Embodiments related to the various methods of the invention are intended to be applied equally to other methods, therapeutic uses or methods, the data storage medium or device, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the number \\ of gaps introduced into the longer sequence \\ in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a metabolite” includes a plurality of such candidate agents and reference to “the polypeptide” includes reference to one or more polypeptides and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows representative OPLS-DA scores plots illustrating discrimination between the non-converter (black square) and early converter (white circle) CIS patients using CSF proteomics features alone, as well as plots representing accuracy, specificity, sensitivity, and cumulate Q².

FIG. 2 shows a graph representing accuracy of 10-fold cross-validated OPLS-DA models at discriminating fast and slow converters with increasing numbers of protein features.

FIG. 3 shows a graph representing accuracy of 10-fold cross-validated OPLS-DA models at discriminating fast and slow converters with increasing numbers of protein features (triangle) including CSF metabolite hits (circle) or serum metabolite hits (square).

FIG. 4 shows a graph representing accuracy of 10-fold cross-validated OPLS-DA models at discriminating fast and slow converters using proteomic features (soma only), metabolomic features (+csf), proteomic and serum metabolomics features (+serum), and proteomics plus clinical chemistry parameters (+clin.chem).

### SEQUENCE LISTING

Where an initial Met amino acid residue or a corresponding initial codon is indicated in any of the following SEQ ID NOs, said residue/codon is optional.

| SEQ ID | | | |
|---|---|---|---|
| NO: | UniProt # | Polypeptide Sequence | Detail |
| 1 | O75582 | MEEEGGSSGGAAGTSADGGDGGEQLLTVKH | KS6A5_HUMAN |
| | Entry version | ELRTANLTGHAEKVGIENFELLKVLGTGAY | Ribosomal protein S6 |
| | 193 | GKVFLVRKISGHDTGKLYAMKVLKKATIVQ | kinase alpha-5 |
| | (5 Jun. 2019) | KAKTTEHTRTERQVLEHIRQSPFLVTLHYA | OS = Homo sapiens |
| | Sequence | FQTETKLHLILDYINGGELFTHLSQRERFT | OX = 9606 |
| | version 1 | EHEVQIYVGEIVLALEHLHKLGIIYRDIKL | GN = RPS6KA5 PE = 1 |
| | (1 Nov. 1998) | ENILLDSNGHVVLTDFGLSKEFVADETERA | SV = 1 |
| | | YSFCGTIEYMAPDIVRGGDSGHDKAVDWWS | |
| | | LGVLMYELLTGASPFTVDGEKNSQAEISRR | |
| | | ILKSEPPYPQEMSALAKDLIQRLLMKDPKK | |
| | | RLGCGPRDADEIKEHLFFQKINWDDLAAKK | |
| | | VPAPFKPVIRDELDVSNFAEEFTEMDPTYS | |
| | | PAALPQSSEKLFQGYSFVAPSILFKRNAAV | |
| | | IDPLQFHMGVERPGVTNVARSAMMKDSPFY | |
| | | QHYDLDLKDKPLGEGSFSICRKCVHKKSNQ | |
| | | AFAVKIISKRMEANTQKEITALKLCEGHPN | |
| | | IVKLHEVFHDQLHTFLVMELLNGGELFERI | |
| | | KKKKHFSETEASYIMRKLVSAVSHMHDVGV | |
| | | VHRDLKPENLLFTDENDNLEIKIIDFGFAR | |
| | | LKPPDNQPLKTPCFTLHYAAPELLNQNGYD | |
| | | ESCDLWSLGVILYTMLSGQVPFQSHDRSLT | |
| | | CTSAVEIMKKIKKGDFSFEGEAWKNVSQEA | |
| | | KDLIQGLLTVDPNKRLKMSGLRYNEWLQDG | |
| | | SQLSSNPLMTPDILGSSGAAVHTCVKATFH | |
| | | AFNKYKREGFCLQNVDKAPLAKRRKMKKTS | |
| | | TSTETRSSSSESSHSSSSHSHGKTTPTKTL | |
| | | QPSNPADSNNPETLFQFSDSVA | |
| 2 | P18887 | MPEIRLRHVVSCSSQDSTHCAENLLKADTY | XRCC1_HUMAN |
| | Entry version | RKWRAAKAGEKTISVVLQLEKEEQIHSVDI | DNA repair protein |
| | 204 | GNDGSAFVEVLVGSSAGGAGEQDYEVLLVT | XRCC1 OS = Homo |
| | (8 May 2019) | SSFMSPSESRSGSNPNRVRMFGPDKLVRAA | sapiens OX = 9606 |
| | Sequence | AEKRWDRVKIVCSQPYSKDSPFGLSFVRFH | GN = XRCC1 PE = 1 |
| | version 2 | SPPDKDEAEAPSQKVTVTKLGQFRVKEEDE | SV = 2 |
| | (11 Jan. 2011) | SANSLRPGALFFSRINKTSPVTASDPAGPS | |
| | | YAAATLQASSAASSASPVSRAIGSTSKPQE | |
| | | SPKGKRKLDLNQEEKKTPSKPPAQLSPSVP | |
| | | KRPKLPAPTRTPATAPVPARAQGAVTGKPR | |
| | | GEGTEPRRPRAGPEELGKILQGVVVVLSGF | |
| | | QNPFRSELRDKALELGAKYRPDWTRDSTHL | |
| | | ICAFANTPKYSQVLGLGGRIVRKEWVLDCH | |
| | | RMRRRLPSRRYLMAGPGSSSEEDEASHSGG | |
| | | SGDEAPKLPQKQPQTKTKPTQAAGPSSPQK | |
| | | PPTPEETKAASPVLQEDIDIEGVQSEGQDN | |
| | | GAEDSGDTEDELRRVAEQKEHRLPPGQEEN | |
| | | GEDPYAGSTDENTDSEEHQEPPDLPVPELP | |
| | | DFFQGKHFFLYGEFPGDERRKLIRYVTAFN | |
| | | GELEDNMSDRVQFVITAQEWDPSFEEALMD | |
| | | NPSLAFVRPRWIYSCNEKQKLLPHQLYGVV | |
| | | PQA | |
| 3 | O00154 | MKLLARALRLCEFGRQASSRRLVAGQGCVG | BACH_HUMAN |
| | Entry version | PRRGCCAPVQVVGPRADLPPCGACITGRIM | Cytosolic acyl |
| | 178 | RPDDANVAGNVHGGTILKMIEEAGAIISTR | coenzyme A thioester |
| | (8 May 2019) | HCNSQNGERCVAALARVERTDFLSPMCIGE | hydrolase OS = Homo |
| | Sequence | VAHVSAEITYTSKHSVEVQVNVMSENILTG | sapiens OX = 9606 |
| | version 3 | AKKLTNKATLWYVPLSLKNVDKVLEVPPVV | GN = ACOT7 PE = 1 |
| | (12 Feb. 2003) | YSRQEQEEEGRKRYEAQKLERMETKWRNGD | SV = 3 |
| | | IVQPVLNPEPNTVSYSQSSLIHLVGPSDCT | |
| | | LHGFVHGGVTMKLMDEVAGIVAARHCKTNI | |
| | | VTASVDAINFHDKIRKGCVITISGRMTFTS | |
| | | NKSMEIEVLVDADPVVDSSQKRYRAASAFF | |
| | | TYVSLSQEGRSLPVPQLVPETEDEKKRFEE | |
| | | GKGRYLQMKAKRQGHAEPQP | |
| 4 | P51813 | MDTKSILEELLLKRSQQKKKMSPNNYKERL | BMX_HUMAN |
| | Entry version | FVLTKTNLSYYEYDKMKRGSRKGSIEIKKI | Cytoplasmic tyrosine- |
| | 194 | RCVEKVNLEEQTPVERQYPFQIVYKDGLLY | protein kinase BMX |
| | (8 May 2019) | VYASNEESRSQWLKALQKEIRGNPHLLVKY | OS = Homo sapiens |
| | Sequence | HSGFFVDGKFLCCQQSCKAAPGCTLWEAYA | OX = 9606 GN = BMX |
| | version 1 | NLHTAVNEEKHRVPTFPDRVLKIPRAVPVL | PE = 1 SV = 1 |
| | (1 Oct. 1996) | KMDAPSSSTTLAQYDNESKKNYGSQPPSSS | |
| | | TSLAQYDSNSKKIYGSQPNFNMQYIPREDF | |
| | | PDWWQVRKLKSSSSSEDVASSNQKERNVNH | |
| | | TTSKISWEFPESSSSEEEENLDDYDWFAGN | |
| | | ISRSQSEQLLRQKGKEGAFMVRNSSQVGMY | |
| | | TVSLFSKAVNDKKGTVKHYHVHTNAENKLY | |
| | | LAENYCFDSIPKLIHYHQHNSAGMITRLRH | |
| | | PVSTKANKVPDSVSLGNGIWELKREEITLL | |
| | | KELGSGQFGVVQLGKWKGQYDVAVKMIKEG | |
| | | SMSEDEFFQEAQTMMKLSHPKLVKFYGVCS | |
| | | KEYPIYIVTEYISNGCLLNYLRSHGKGLEP | |
| | | SQLLEMCYDVCEGMAFLESHQFIHRDLAAR | |
| | | NCLVDRDLCVKVSDFGMTRYVLDDQYVSSV | |
| | | GTKFPVKWSAPEVFHYFKYSSKSDVWAFGI | |
| | | LMWEVFSLGKQPYDLYDNSQVVLKVSQGHR | |
| | | LYRPHLASDTIYQIMYSCWHELPEKRPTFQ | |
| | | QLLSSIEPLREKDKH | |
| 5 | P43235 | MWGLKVLLLPVVSFALYPEEILDTHWELWK | CATK_HUMAN |
| | Entry version | KTHRKQYNNKVDEISRRLIWEKNLKYISIH | Cathepsin K |
| | 193 | NLEASLGVHTYELAMNHLGDMTSEEVVQKM | OS = Homo sapiens |
| | (5 Jun. 2019) | TGLKVPLSHSRSNDTLYIPEWEGRAPDSVD | OX = 9606 GN = CTSK |
| | Sequence | YRKKGYVTPVKNQGQCGSCWAFSSVGALEG | PE = 1 SV = 1 |
| | version 1 | QLKKKTGKLLNLSPQNLVDCVSENDGCGGG | |
| | (1 Nov. 1995) | YMTNAFQYVQKNRGIDSEDAYPYVGQEESC | |
| | | MYNPTGKAAKCRGYREIPEGNEKALKRAVA | |
| | | RVGPVSVAIDASLTSFQFYSKGVYYDESCN | |
| | | SDNLNHAVLAVGYGIQKGNKHWIIKNSWGE | |
| | | NWGNKGYILMARNKNNACGIANLASFPKM | |
| 6 | P06753 | MMEAIKKKMQMLKLDKENALDRAEQAEAEQ | TPM3_HUMAN |
| | Entry version | KQAEERSKQLEDELAAMQKKLKGTEDELDK | Tropomyosin alpha-3 |
| | 217 | YSEALKDAQEKLELAEKKAADAEAEVASLN | chain OS = Homo |
| | (5 Jun. 2019) | RRIQLVEEELDRAQERLATALQKLEEAEKA | sapiens OX = 9606 |
| | Sequence | ADESERGMKVIENRALKDEEKMELQEIQLK | GN = TPM3 PE = 1 |
| | version 2 | EAKHIAEEADRKYEEVARKLVIIEGDLERT | SV = 2 |
| | (26 Jun. 2013) | EERAELAESKCSELEEELKNVTNNLKSLEA | |
| | | QAEKYSQKEDKYEEEIKILTDKLKEAETRA | |
| | | EFAERSVAKLEKTIDDLEDELYAQKLKYKA | |
| | | ISEELDHALNDMTSI | |
| 7 | Q92974 | MSRIESLTRARIDRSRELASKTREKEKMKE | ARHG2_HUMAN Rho |
| | Entry version | AKDARYTNGHLFTTISVSGMTMCYACNKSI | guanine nucleotide |
| | 194 | TAKEALICPTCNVTIHNRCKDTLANCTKVK | exchange factor 2 |
| | (5 Jun. 2019) | QKQQKAALLKNNTALQSVSLRSKTTIRERP | OS = Homo sapiens |
| | Sequence | SSAIYPSDSFRQSLLGSRRGRSSLSLAKSV | OX = 9606 |
| | version 4 | STTNIAGHFNDESPLGLRRILSQSTDSLNM | GN = ARHGEF2 PE = 1 |
| | (22 Jul. 2008) | RNRTLSVESLIDEAEVIYSELMSDFEMDEK | SV = 4 |
| | | DFAADSWSLAVDSSFLQQHKKEVMKQQDVI | |
| | | YELIQTELHHVRTLKIMTRLFRTGMLEELH | |
| | | LEPGVVQGLFPCVDELSDIHTRFLSQLLER | |
| | | RRQALCPGSTRNFVIHRLGDLLISQFSGPS | |
| | | AEQMCKTYSEFCSRHSKALKLYKELYARDK | |
| | | RFQQFIRKVTRPAVLKRHGVQECILLVTQR | |
| | | ITKYPLLISRILQHSHGIEEERQDLTTALG | |
| | | LVKELLSNVDEGIYQLEKGARLQEIYNRMD | |
| | | PRAQTPVPGKGPFGREELLRRKLIHDGCLL | |
| | | WKTATGRFKDVLVLLMTDVLVFLQEKDQKY | |
| | | IFPTLDKPSVVSLQNLIVRDIANQEKGMFL | |
| | | ISAAPPEMYEVHTASRDDRSTWIRVIQQSV | |
| | | RTCPSREDFPLIETEDEAYLRRIKMELQQK | |
| | | DRALVELLREKVGLFAEMTHFQAEEDGGSG | |
| | | MALPTLPRGLFRSESLESPRGERLLQDAIR | |
| | | EVEGLKDLLVGPGVELLLTPREPALPLEPD | |
| | | SGGNTSPGVTANGEARTFNGSIELCRADSD | |
| | | SSQRDRNGNQLRSPQEEALQRLVNLYGLLH | |
| | | GLQAAVAQQDTLMEARFPEGPERREKLCRA | |
| | | NSRDGEAGRAGAAPVAPEKQATELALLQRQ | |
| | | HALLQEELRRCRRLGEERATEAGSLEARLR | |
| | | ESEQARALLEREAEEARRQLAALGQTEPLP | |
| | | AEAPWARRPVDPRRRSLPAGDALYLSFNPP | |
| | | QPSRGTDRLDLPVTTRSVHRNFEDRERQEL | |
| | | GSPEERLQDSSDPDTGSEEEGSSRLSPPHS | |
| | | PRDFTRMQDIPEETESRDGEAVASES | |
| 8 | Q9HB21 | MPYVDRQNRICGFLDIEENENSGKFLRRYF | PKHA1_HUMAN |
| | Entry version | ILDTREDSFVWYMDNPQNLPSGSSRVGAIK | Pleckstrin homology |
| | 150 | LTYISKVSDATKLRPKAEFCFVMNAGMRKY | domain-containing |
| | (8 May 2019) | FLQANDQQDLVEWVNVLNKAIKITVPKQSD | family A member 1 |
| | Sequence | SQPNSDNLSRHGECGKKQVSYRTDIVGGVP | OS = Homo sapiens |
| | version 2 | IITPTQKEEVNECGESIDRNNLKRSQSHLP | OX = 9606 |
| | (29 Mar. 2004) | YFTPKPPQDSAVIKAGYCVKQGAVMKNWKR | GN = PLEKHA1 PE = 1 |
| | | RYFQLDENTIGYFKSELEKEPLRVIPLKEV | SV = 2 |
| | | HKVQECKQSDIMMRDNLFEIVTTSRTFYVQ | |
| | | ADSPEEMHSWIKAVSGAIVAQRGPGRSASS | |
| | | EHPPGPSESKHAFRPTNAATATSHSTASRS | |
| | | NSLVSTFTMEKRGFYESLAKVKPGNFKVQT | |
| | | VSPREPASKVTEQALLRPQSKNGPQEKDCD | |
| | | LVDLDDASLPVSDV | |
| 9 | Q8IYU8 | MAAAAGSCARVAAWGGKLRRGLAVSRQAVR | MICU2_HUMAN |
| | Entry version | SPGPLAAAVAGAALAGAGAAWHHSRVSVAA | Calcium uptake |
| | 138 | RDGSFTVSAQKNVEHGIIYIGKPSLRKQRF | protein 2, |
| | (8 May 2019) | MQFSSLEHEGEYYMTPRDFLFSVMFEQMER | mitochondrial |
| | Sequence | KTSVKKLTKKDIEDTLSGIQTAGCGSTFFR | OS = Homo sapiens |
| | version 2 | DLGDKGLISYTEYLFLLTILTKPHSGFHVA | OX = 9606 GN = MICU2 |
| | (3 Oct. 2006) | FKMLDTDGNEMIEKREFFKLQKIISKQDDL | PE = 1 SV = 2 |
| | | MTVKTNETGYQEAIVKEPEINTTLQMRFFG | |
| | | KRGQRKLHYKEFRRFMENLQTEIQEMEFLQ | |
| | | FSKGLSFMRKEDFAEWLLFFTNTENKDIYW | |
| | | KNVREKLSAGESISLDEFKSFCHFTTHLED | |
| | | FAIAMQMFSLAHRPVRLAEFKRAVKVATGQ | |
| | | ELSNNILDTVFKIFDLDGDECLSHEEFLGV | |
| | | LKNRMHRGLWVPQHQSIQEYWKCVKKESIK | |
| | | GVKEVWKQAGKGLF | |
| 10 | Q6ZSG1 | MVLVHVGYLVLPVFGSVRNRGAPFQRSQHP | RN165_HUMAN E3 |
| | Entry version | HATSCRHFHLGPPQPQQLAPDFPLAHPVQS | ubiquitin-protein |
| | 127 | QPGLSAHMAPAHQHSGALHQSLTPLPTLQF | ligase RNF165 |
| | (8 May 2019) | QDVTGPSFLPQALHQQYLLQQQLLEAQHRR | OS = Homo sapiens |
| | Sequence | LVSHPRRSQERVSVHPHRLHPSFDFGQLQT | OX = 9606 |
| | version 1 | PQPRYLAEGTDWDLSVDAGLSPAQFQVRPI | GN = RNF165 PE = 1 |
| | (5 Jul. 2004) | PQHYQHYLATPRMHHFPRNSSSTQMVVHEI | SV = 1 |
| | | RNYPYPQLHFLALQGLNPSRHTSAVRESYE | |
| | | ELLQLEDRLGNVTRGAVQNTIERFTFPHKY | |
| | | KKRRPQDGKGKKDEGEESDTDEKCTICLSM | |
| | | LEDGEDVRRLPCMHLFHQLCVDQWLAMSKK | |
| | | CPICRVDIETQLGADS | |
| 11 | O76036 | MSSTLPALLCVGLCLSQRISAQQQTLPKPF | NCTR1_HUMAN |
| | Entry version | IWAEPHFMVPKEKQVTICCQGNYGAVEYQL | Natural cytotoxicity |
| | 160 | HFEGSLFAVDRPKPPERINKVKFYIPDMNS | triggering receptor 1 |
| | (8 May 2019) | RMAGQYSCIYRVGELWSEPSNLLDLVVTEM | OS = Homo sapiens |
| | Sequence | YDTPTLSVHPGPEVISGEKVTFYCRLDTAT | OX = 9606 GN = NCR1 |
| | version 1 | SMFLLLKEGRSSHVQRGYGKVQAEFPLGPV | PE = 1 SV = 1 |
| | (1 Nov. 1998) | TTAHRGTYRCFGSYNNHAWSFPSEPVKLLV | |
| | | TGDIENTSLAPEDPTFPADTWGTYLLTTET | |
| | | GLQKDHALWDHTAQNLLRMGLAFLVLVALV | |
| | | WFLVEDWLSRKRTRERASRASTWEGRRRLN | |
| | | TQTL | |
| 12 | O75843 | MVVPSLKLQDLIEEIRGAKTQAQEREVIQK | AP1G2_HUMAN AP- |
| | Entry version | ECAHIRASFRDGDPVHRHRQLAKLLYVHML | 1 complex subunit |
| | 165 | GYPAHFGQMECLKLIASSRFTDKRVGYLGA | gamma-like 2 |
| | (8 May 2019) | MLLLDERHDAHLLITNSIKNDLSQGIQPVQ | OS = Homo sapiens |
| | Sequence | GLALCTLSTMGSAEMCRDLAPEVEKLLLQP | OX = 9606 |
| | version 1 | SPYVRKKAILTAVHMIRKVPELSSVFLPPC | GN = AP1G2 PE = 1 |
| | (1 Nov. 1998) | AQLLHERHHGILLGTITLITELCERSPAAL | SV = 1 |
| | | RHFRKVVPQLVHILRTLVTMGYSTEHSISG | |
| | | VSDPFLQVQILRLLRILGRNHEESSETMND | |
| | | LLAQVATNTDTSRNAGNAVLFETVLTIMDI | |
| | | RSAAGLRVLAVNILGRFLLNSDRNIRYVAL | |
| | | TSLLRLVQSDHSAVQRHRPTVVECLRETDA | |
| | | SLSRRALELSLALVNSSNVRAMMQELQAFL | |
| | | ESCPPDLRADCASGILLAAERFAPTKRWHI | |
| | | DTILHVLTTAGTHVRDDAVANLTQLIGGAQ | |
| | | ELHAYSVRRLYNALAEDISQQPLVQVAAWC | |
| | | IGEYGDLLLAGNCEEIEPLQVDEEEVLALL | |
| | | EKVLQSHMSLPATRGYALTALMKLSTRLCG | |
| | | DNNRIRQVVSIYGSCLDVELQQRAVEYDTL | |
| | | FRKYDHMRAAILEKMPLVERDGPQADEEAK | |
| | | ESKEAAQLSEAAPVPTEPQASQLLDLLDLL | |
| | | DGASGDVQHPPHLDPSPGGALVHLLDLPCV | |
| | | PPPPAPIPDLKVFEREGVQLNLSFIRPPEN | |
| | | PALLLITITATNFSEGDVTHFICQAAVPKS | |
| | | LQLQLQAPSGNTVPARGGLPITQLFRILNP | |
| | | NKAPLRLKLRLTYDHFHQSVQEIFEVNNLP | |
| | | VESWQ | |
| 13 | Q14914 | MVRTKTWTLKKHFVGYPTNSDFELKTAELP | PTGR1_HUMAN |
| | Entry version | PLKNGEVLLEALFLTVDPYMRVAAKRLKEG | Prostaglandin |
| | 181 | DTMMGQQVAKVVESKNVALPKGTIVLASPG | reductase 1 |
| | (8 May 2019) | WTTHSISDGKDLEKLLTEWPDTIPLSLALG | OS = Homo sapiens |
| | Sequence | TVGMPGLTAYFGLLEICGVKGGETVMVNAA | OX = 9606 |
| | version 2 | AGAVGSVVGQIAKLKGCKVVGAVGSDEKVA | GN = PTGR1 PE = 1 |
| | (19 Sep. 2002) | YLQKLGFDVVFNYKTVESLEETLKKASPDG | SV = 2 |
| | | YDCYFDNVGGEFSNTVIGQMKKFGRIAICG | |
| | | AISTYNRTGPLPPGPPPEIVIYQELRMEAF | |
| | | VVYRWQGDARQKALKDLLKWVLEGKIQYKE | |
| | | YIIEGFENMPAAFMGMLKGDNLGKTIVKA | |
| 14 | Q01344 | MIIVAHVLLILLGATEILQADLLPDEKISL | IL5RA_HUMAN |
| | Entry version | LPPVNFTIKVTGLAQVLLQWKPNPDQEQRN | Interleukin-5 receptor |
| | 188 | VNLEYQVKINAPKEDDYETRITESKCVTIL | subunit alpha |
| | (5 Jun. 2019) | HKGFSASVRTILQNDHSLLASSWASAELHA | OS = Homo sapiens |
| | Sequence | PPGSPGTSIVNLTCTTNTTEDNYSRLRSYQ | OX = 9606 GN = IL5RA |
| | version 2 | VSLHCTWLVGTDAPEDTQYFLYYRYGSWTE | PE = 1 SV = 2 |
| | (17 Oct. 2006) | ECQEYSKDTLGRNIACWFPRTFILSKGRDW | |
| | | LAVLVNGSSKHSAIRPFDQLFALHAIDQIN | |
| | | PPLNVTAEIEGTRLSIQWEKPVSAFPIHCF | |
| | | DYEVKIHNTRNGYLQIEKLMTNAFISIIDD | |
| | | LSKYDVQVRAAVSSMCREAGLWSEWSQPIY | |
| | | VGNDEHKPLREWFVIVIMATICFILLILSL | |
| | | ICKICHLWIKLFPPIPAPKSNIKDLFVTTN | |
| | | YEKAGSSETEIEVICYIEKPGVETLEDSVF | |
| 15 | Q96PF2 | MDDATVLRKKGYIVGINLGKGSYAKVKSAY | TSSK2_HUMAN |
| | Entry version | SERLKFNVAVKIIDRKKTPTDFVERFLPRE | Testis-specific |
| | 172 | MDILATVNHGSIIKTYEIFETSDGRIYIIM | serine/threonine- |
| | (8 May 2019) | ELGVQGDLLEFIKCQGALHEDVARKMFRQL | protein kinase 2 |
| | Sequence | SSAVKYCHDLDIVHRDLKCENLLLDKDFNI | OS = Homo sapiens |
| | version 2 | KLSDFGFSKRCLRDSNGRIILSKTFCGSAA | OX = 9606 GN = TSSK2 |
| | (30 Apr. 2003) | YAAPEVLQSIPYQPKVYDIWSLGVILYIMV | PE = 1 SV = 2 |
| | | CGSMPYDDSDIRKMLRIQKEHRVDFPRSKN | |
| | | LTCECKDLIYRMLQPDVSQRLHIDEILSHS | |
| | | WLQPPKPKATSSASFKREGEGKYRAECKLD | |
| | | TKTGLRPDHRPDHKLGAKTQHRLLVVPENE | |
| | | NRMEDRLAETSRAKDHHISGAEVGKAST | |
| 16 | P51451 | MGLVSSKKPDKEKPIKEKDKGQWSPLKVSA | BLK_HUMAN |
| | Entry version | QDKDAPPLPPLVVFNHLTPPPPDEHLDEDK | Tyrosine-protein |
| | 181 | HFVVALYDYTAMNDRDLQMLKGEKLQVLKG | kinase Blk OS = Homo |
| | (8 May 2019) | TGDWWLARSLVTGREGYVPSNFVARVESLE | sapiens OX = 9606 |
| | Sequence | MERWFFRSQGRKEAERQLLAPINKAGSFLI | GN = BLK PE = 1 SV = 3 |
| | version 3 | RESETNKGAFSLSVKDVTTQGELIKHYKIR | |
| | (21 Aug. 2007) | CLDEGGYYISPRITFPSLQALVQHYSKKGD | |
| | | GLCQRLTLPCVRPAPQNPWAQDEWEIPRQS | |
| | | LRLVRKLGSGQFGEVWMGYYKNNMKVAIKT | |
| | | LKEGTMSPEAFLGEANVMKALQHERLVRLY | |
| | | AVVTKEPIYIVTEYMARGCLLDFLKTDEGS | |
| | | RLSLPRLIDMSAQIAEGMAYIERMNSIHRD | |
| | | LRAANILVSEALCCKIADFGLARIIDSEYT | |
| | | AQEGAKFPIKWTAPEAIHFGVFTIKADVWS | |
| | | FGVLLMEVVTYGRVPYPGMSNPEVIRNLER | |
| | | GYRMPRPDTCPPELYRGVIAECWRSRPEER | |
| | | PTFEFLQSVLEDFYTATERQYELQP | |
| 17 | P01374 | MTPPERLFLPRVCGTTLHLLLLGLLLVLLP | TNFB_HUMAN |
| | Entry version | GAQGLPGVGLTPSAAQTARQHPKMHLAHST | Lymphotoxin-alpha |
| | 202 | LKPAAHLIGDPSKQNSLLWRANTDRAFLQD | OS = Homo sapiens |
| | (8 May 2019) | GFSLSNNSLLVPTSGIYFVYSQVVFSGKAY | OX = 9606 GN = LTA |
| | Sequence | SPKATSSPLYLAHEVQLFSSQYPFHVPLLS | PE = 1 SV = 2 |
| | version 2 | SQKMVYPGLQEPWLHSMYHGAAFQLTQGDQ | |
| | (1 Jul. 1989) | LSTHTDGIPHLVLSPSTVFFGAFAL | |
| 18 | Q06643 | MGALGLEGRGGRLQGRGSLLLAVAGATSLV | TNFC_HUMAN |
| | Entry version | TLLLAVPITVLAVLALVPQDQGGLVTETAD | Lymphotoxin-beta |
| | 172 | PGAQAQQGLGFQKLPEEEPETDLSPGLPAA | OS = Homo sapiens |
| | (8 May 2019) | HLIGAPLKGQGLGWETTKEQAFLTSGTQFS | OX = 9606 GN = LTB |
| | Sequence | DAEGLALPQDGLYYLYCLVGYRGRAPPGGG | PE = 1 SV = 1 |
| | version 1 | DPQGRSVTLRSSLYRAGGAYGPGTPELLLE | |
| | (1 Jun. 1994) | GAETVTPVLDPARRQGYGPLWYTSVGFGGL | |
| | | VQLRRGERVYVNISHPDMVDFARGKTFFGA | |
| | | VMVG | |
| 19 | Q7Z434 | MPFAEDKTYKYICRNFSNFCNVDVVEILPY | MAVS_HUMAN |
| | Entry version | LPCLTARDQDRLRATCTLSGNRDTLWHLFN | Mitochondrial |
| | 167 | TLQRRPGWVEYFIAALRGCELVDLADEVAS | antiviral-signaling |
| | (5 Jun. 2019) | VYQSYQPRTSDRPPDPLEPPSLPAERPGPP | protein OS = Homo |
| | Sequence | TPAAAHSIPYNSCREKEPSYPMPVQETQAP | sapiens OX = 9606 |
| | version 2 | ESPGENSEQALQTLSPRAIPRNPDGGPLES | GN = MAVS PE = 1 |
| | (10 May 2004) | SSDLAALSPLTSSGHQEQDTELGSTHTAGA | SV = 2 |
| | | TSSLTPSRGPVSPSVSFQPLARSTPRASRL | |
| | | PGPTGSVVSTGTSFSSSSPGLASAGAAEGK | |
| | | QGAESDQAEPIICSSGAEAPANSLPSKVPT | |
| | | TLMPVNTVALKVPANPASVSTVPSKLPTSS | |
| | | KPPGAVPSNALTNPAPSKLPINSTRAGMVP | |
| | | SKVPTSMVLTKVSASTVPTDGSSRNEETPA | |
| | | APTPAGATGGSSAWLDSSSENRGLGSELSK | |
| | | PGVLASQVDSPFSGCFEDLAISASTSLGMG | |
| | | PCHGPEENEYKSEGTFGIHVAENPSIQLLE | |
| | | GNPGPPADPDGGPRPQADRKFQEREVPCHR | |
| | | PSPGALWLQVAVTGVLVVTLLVVLYRRRLH | |
| 20 | P30793 | MEKGPVRAPAEKPRGARCSNGFPERDPPRP | GCH1_HUMAN GTP |
| | Entry version | GPSRPAEKPPRPEAKSAQPADGWKGERPRS | cyclohydrolase 1 |
| | 201 | EEDNELNLPNLAAAYSSILSSLGENPQRQG | OS = Homo sapiens |
| | (8 May 2019) | LLKTPWRAASAMQFFTKGYQETISDVLNDA | OX = 9606 GN = GCH1 |
| | Sequence | IFDEDHDEMVIVKDIDMFSMCEHHLVPFVG | PE = 1 SV = 1 |
| | version 1 | KVHIGYLPNKQVLGLSKLARIVEIYSRRLQ | |
| | (1 Jul. 1993) | VQERLTKQIAVAITEALRPAGVGVVVEATH | |
| | | MCMVMRGVQKMNSKTVTSTMLGVFREDPKT | |
| | | REEFLTLIRS | |
| 21 | P48745 | MQSVQSTSFCLRKQCLCLTFLLLHLLGQVA | CCN3_HUMAN CCN |
| | Entry version | ATQRCPPQCPGRCPATPPTCAPGVRAVLDG | family member 3 |
| | 161 | CSCCLVCARQRGESCSDLEPCDESSGLYCD | OS = Homo sapiens |
| | (8 May 2019) | RSADPSNQTGICTAVEGDNCVFDGVIYRSG | OX = 9606 GN = CCN3 |
| | Sequence | EKFQPSCKFQCTCRDGQIGCVPRCQLDVLL | PE = 1 SV = 1 |
| | version 1 | PEPNCPAPRKVEVPGECCEKWICGPDEEDS | |
| | (1 Feb. 1996) | LGGLTLAAYRPEATLGVEVSDSSVNCIEQT | |
| | | TEWTACSKSCGMGFSTRVTNRNRQCEMLKQ | |
| | | TRLCMVRPCEQEPEQPTDKKGKKCLRTKKS | |
| | | LKAIHLQFKNCTSLHTYKPRFCGVCSDGRC | |
| | | CTPHNTKTIQAEFQCSPGQIVKKPVMVIGT | |
| | | CTCHTNCPKNNEAFLQELELKTTRGKM | |
| 22 | Q08AG7 | MASSSGAGAAAAAAAANLNAVRETMDVLLE | MZT1_HUMAN |
| | Entry version | ISRILNTGLDMETLSICVRLCEQGINPEAL | Mitotic-spindle |
| | 94 | SSVIKELRKATEALKAAENMTS | organizing protein 1 |
| | (8 May 2019) | | OS = Homo sapiens |
| | Sequence | | OX = 9606 GN = MZT1 |
| | version 2 | | PE = 1 SV = 2 |
| | (20 May 2008) | | |
| 23 | O15068 | MFDCWRFILCKRPGSNSYSSPQRPNEAKKE | MCF2L_HUMAN |
| | Entry version | ETDHQIDVSDVIRLVQDTPEATAMATDEIM | Guanine nucleotide |
| | 180 | HQDIVPLCAADIQDQLKKRFAYLSGGRGQD | exchange factor DBS |
| | (8 May 2019) | GSPVITFPDYPAFSEIPDKEFQNVMTYLTS | OS = Homo sapiens |
| | Sequence | IPSLQDAGIGFILVIDRRRDKWTSVKASVL | OX = 9606 |
| | version 2 | RIAASFPANLQLVLVLRPTGFFQRTLSDIA | GN = MCF2L PE = 1 |
| | (12 Jun. 2007) | FKFNRDDFKMKVPVIMLSSVPDLHGYIDKS | SV = 2 |
| | | QLTEDLGGTLDYCHSRWLCQRTAIESFALM | |
| | | VKQTAQMLQSFGTELAETELPNDVQSTSSV | |
| | | LCAHTEKKDKAKEDLRLALKEGHSVLESLR | |
| | | ELQAEGSEPSVNQDQLDNQATVQRLLAQLN | |
| | | ETEAAFDEFWAKHQQKLEQCLQLRHFEQGF | |
| | | REVKAILDAASQKIATFTDIGNSLAHVEHL | |
| | | LRDLASFEEKSGVAVERARALSLDGEQLIG | |
| | | NKHYAVDSIRPKCQELRHLCDQFSAEIARR | |
| | | RGLLSKSLELHRRLETSMKWCDEGIYLLAS | |
| | | QPVDKCQSQDGAEAALQEIEKFLETGAENK | |
| | | IQELNAIYKEYESILNQDLMEHVRKVFQKQ | |
| | | ASMEEVFHRRQASLKKLAARQTRPVQPVAP | |
| | | RPEALAKSPCPSPGIRRGSENSSSEGGALR | |
| | | RGPYRRAKSEMSESRQGRGSAGEEEESLAI | |
| | | LRRHVMSELLDTERAYVEELLCVLEGYAAE | |
| | | MDNPLMAHLLSTGLHNKKDVLFGNMEEIYH | |
| | | FHNRIFLRELENYTDCPELVGRCFLERMED | |
| | | FQIYEKYCQNKPRSESLWRQCSDCPFFQEC | |
| | | QRKLDHKLSLDSYLLKPVQRITKYQLLLKE | |
| | | MLKYSRNCEGAEDLQEALSSILGILKAVND | |
| | | SMHLIAITGYDGNLGDLGKLLMQGSFSVWT | |
| | | DHKRGHTKVKELARFKPMQRHLFLHEKAVL | |
| | | FCKKREENGEGYEKAPSYSYKQSLNMAAVG | |
| | | ITENVKGDAKKFEIWYNAREEVYIVQAPTP | |
| | | EIKAAWVNEIRKVLTSQLQACREASQHRAL | |
| | | EQSQSLPLPAPTSTSPSRGNSRNIKKLEER | |
| | | KTDPLSLEGYVSSAPLTKPPEKGKGWSKTS | |
| | | HSLEAPEDDGGWSSAEEQINSSDAEEDGGL | |
| | | GPKKLVPGKYTVVADHEKGGPDALRVRSGD | |
| | | VVELVQEGDEGLWYVRDPTTGKEGWVPASS | |
| | | LSVRLGPSGSAQCLSSSGKAHVPRAHP | |
| 24 | P19320 | MPGKMVVILGASNILWIMFAASQAFKIETT | VCAM1_HUMAN |
| | Entry version | PESRYLAQIGDSVSLTCSTTGCESPFFSWR | Vascular cell |
| | 232 | TQIDSPLNGKVTNEGTTSTLTMNPVSFGNE | adhesion protein 1 |
| | (8 May 2019) | HSYLCTATCESRKLEKGIQVEIYSFPKDPE | OS = Homo sapiens |
| | Sequence | IHLSGPLEAGKPITVKCSVADVYPFDRLEI | OX = 9606 |
| | version 1 | DLLKGDHLMKSQEFLEDADRKSLETKSLEV | GN = VCAM1 PE = 1 |
| | (1 Nov. 1990) | TFTPVIEDIGKVLVCRAKLHIDEMDSVPTV | SV = 1 |
| | | RQAVKELQVYISPKNTVISVNPSTKLQEGG | |
| | | SVTMTCSSEGLPAPEIFWSKKLDNGNLQHL | |
| | | SGNATLTLIAMRMEDSGIYVCEGVNLIGKN | |
| | | RKEVELIVQEKPFTVEISPGPRIAAQIGDS | |
| | | VMLTCSVMGCESPSFSWRTQIDSPLSGKVR | |
| | | SEGTNSTLTLSPVSFENEHSYLCTVTCGHK | |
| | | KLEKGIQVELYSFPRDPEIEMSGGLVNGSS | |
| | | VTVSCKVPSVYPLDRLEIELLKGETILENI | |
| | | EFLEDTDMKSLENKSLEMTFIPTIEDTGKA | |
| | | LVCQAKLHIDDMEFEPKQRQSTQTLYVNVA | |
| | | PRDTTVLVSPSSILEEGSSVNMTCLSQGFP | |
| | | APKILWSRQLPNGELQPLSENATLTLISTK | |
| | | MEDSGVYLCEGINQAGRSRKEVELIIQVTP | |
| | | KDIKLTAFPSESVKEGDTVIISCTCGNVPE | |
| | | TWIILKKKAETGDTVLKSIDGAYTIRKAQL | |
| | | KDAGVYECESKNKVGSQLRSLTLDVQGREN | |
| | | NKDYFSPELLVLYFASSLIIPAIGMIIYFA | |
| | | RKANMKGSYSLVEAQKSKV | |
| 25 | P16152 | MSSGIHVALVTGGNKGIGLAIVRDLCRLFS | CBR1_HUMAN |
| | Entry version | GDVVLTARDVTRGQAAVQQLQAEGLSPRFH | Carbonyl reductase |
| | 207 | QLDIDDLQSIRALRDFLRKEYGGLDVLVNN | [NADPH] 1 |
| | (8 May 2019) | AGIAFKVADPTPFHIQAEVTMKTNFFGTRD | OS = Homo sapiens |
| | Sequence | VCTELLPLIKPQGRVVNVSSIMSVRALKSC | OX = 9606 GN = CBR1 |
| | version 3 | SPELQQKFRSETITEEELVGLMNKFVEDTK | PE = 1 SV = 3 |
| | (23 Jan. 2007) | KGVHQKEGWPSSAYGVTKIGVTVLSRIHAR | |
| | | KLSEQRKGDKILLNACCPGWVRTDMAGPKA | |
| | | TKSPEEGAETPVYLALLPPDAEGPHGQFVS | |
| | | EKRVEQW | |
| 26 | Q6JVE6 | MRQGLLVLALVLVLVLVLAAGSQVQEWYPR | LCN10_HUMAN |
| | Entry version | ESHALNWNKFSGFWYILATATDAQGFLPAR | Epididymal-specific |
| | 122 | DKRKLGASVVKVNKVGQLRVLLAFRRGQGC | lipocalin-10 |
| | (5 Jun. 2019) | GRAQPRHPGTSGHLWASLSVKGVKAFHVLS | OS = Homo sapiens |
| | Sequence | TDYSYGLVYLRLGRATQNYKNLLLFHRQNV | OX = 9606 GN = LCN10 |
| | version 1 | SSFQSLKEFMDACDILGLSKAAVILPKDAS | PE = 1 SV = 1 |
| | (5 Jul. 2004) | RTHTILP | |
| 27 | Q76M96 | MTWRMGPRFTMLLAMWLVCGSEPHPHATIR | CCD80_HUMAN |
| | Entry version | GSHGGRKVPLVSPDSSRPARFLRHTGRSRG | Coiled-coil domain- |
| | 122 | IERSTLEEPNLQPLQRRRSVPVLRLARPTE | containing protein 80 |
| | (8 May 2019) | PPARSDINGAAVRPEQRPAARGSPREMIRD | OS = Homo sapiens |
| | Sequence | EGSSARSRMLRFPSGSSSPNILASFAGKNR | OX = 9606 |
| | version 1 | VWVISAPHASEGYYRLMMSLLKDDVYCELA | GN = CCDC80 PE = 1 |
| | (5 Jul. 2004) | ERHIQQIVLFHQAGEEGGKVRRITSEGQIL | SV = 1 |
| | | EQPLDPSLIPKLMSFLKLEKGKFGMVLLKK | |
| | | TLQVEERYPYPVRLEAMYEVIDQGPIRRIE | |
| | | KIRQKGFVQKCKASGVEGQVVAEGNDGGGG | |
| | | AGRPSLGSEKKKEDPRRAQVPPTRESRVKV | |
| | | LRKLAATAPALPQPPSTPRATTLPPAPATT | |
| | | VTRSTSRAVTVAARPMTTTAFPTTQRPWTP | |
| | | SPSHRPPTTTEVITARRPSVSENLYPPSRK | |
| | | DQHRERPQTTRRPSKATSLESFTNAPPTTI | |
| | | SEP5TRAAGPGRFRDNRMDRREHGHRDPNV | |
| | | VPGPPKPAKEKPPKKKAQDKILSNEYEEKY | |
| | | DLSRPTASQLEDELQVGNVPLKKAKESKKH | |
| | | EKLEKPEKEKKKKMKNENADKLLKSEKQMK | |
| | | KSEKKSKQEKEKSKKKKGGKTEQDGYQKPT | |
| | | NKHFTQSPKKSVADLLGSFEGKRRLLLITA | |
| | | PKAENNMYVQQRDEYLESFCKMATRKISVI | |
| | | TIFGPVNNSTMKIDHFQLDNEKPMRVVDDE | |
| | | DLVDQRLISELRKEYGMTYNDFFMVLTDVD | |
| | | LRVKQYYEVPITMKSVFDLIDTFQSRIKDM | |
| | | EKQKKEGIVCKEDKKQSLENFLSRFRWRRR | |
| | | LLVISAPNDEDWAYSQQLSALSGQACNFGL | |
| | | RHITILKLLGVGEEVGGVLELFPINGSSVV | |
| | | EREDVPAHLVKDIRNYFQVSPEYFSMLLVG | |
| | | KDGNVKSWYPSPMWSMVIVYDLIDSMQLRR | |
| | | QEMAIQQSLGMRCPEDEYAGYGYHSYHQGY | |
| | | QDGYQDDYRHHESYHHGYPY | |
| 28 | P28676 | MAYPGYGGGFGNFSIQVPGMQMGQPVPETG | GRAN_HUMAN |
| | Entry version | PAILLDGYSGPAYSDTYSSAGDSVYTYFSA | Grancalcin OS = Homo |
| | 172 | VAGQDGEVDAEELQRCLTQSGINGTYSPFS | sapiens OX = 9606 |
| | (8 May 2019) | LETCRIMIAMLDRDHTGKMGFNAFKELWAA | GN = GCA PE = 1 SV = 2 |
| | Sequence | LNAWKENFMTVDQDGSGTVEHHELRQAIGL | |
| | version 2 | MGYRLSPQTLTTIVKRYSKNGRIFFDDYVA | |
| | (1 Nov. 1995) | CCVKLRALTDFFRKRDHLQQGSANFIYDDF | |
| | | LQGTMAI | |
| 29 | Q8N428 | MRKIRANAIAILTVAWILGTFYYLWQDNRA | GLT16_HUMAN |
| | Entry version | HAASSGGRGAQRAGRRSEQLREDRTIPLIV | Polypeptide N- |
| | 144 | TGTPSKGFDEKAYLSAKQLKAGEDPYRQHA | acetylgalactosaminyl- |
| | (8 May 2019) | FNQLESDKLSPDRPIRDTRHYSCPSVSYSS | transferase 16 |
| | Sequence | DLPATSVIITFHNEARSTLLRTVKSVLNRT | OS = Homo sapiens |
| | version 2 | PANLIQEIILVDDFSSDPEDCLLLTRIPKV | OX = 9606 |
| | (16 Aug. 2004) | KCLRNDRREGLIRSRVRGADVAAATVLTFL | GN = GALNT16 PE = 1 |
| | | DSHCEVNTEWLPPMLQRVKEDHTRVVSPII | SV = 2 |
| | | DVISLDNFAYLAASADLRGGFDWSLHFKWE | |
| | | QIPLEQKMTRTDPTRPIRTPVIAGGIFVID | |
| | | KSWFNHLGKYDAQMDIWGGENFELSFRVWM | |
| | | CGGSLEIVPCSRVGHVFRKRHPYNFPEGNA | |
| | | LTYIRNTKRTAEVWMDEYKQYYYEARPSAI | |
| | | GKAFGSVATRIEQRKKMNCKSFRWYLENVY | |
| | | PELTVPVKEALPGIIKQGVNCLESQGQNTA | |
| | | GDFLLGMGICRGSAKNPQPAQAWLFSDHLI | |
| | | QQQGKCLAATSTLMSSPGSPVILQMCNPRE | |
| | | GKQKWRRKGSFIQHSVSGLCLETKPAQLVT | |
| | | SKCQADAQAQQWQLLPHT | |
| 30 | P08603 | MRLLAKIICLMLWAICVAEDCNELPPRRNT | CFAH_HUMAN |
| | Entry version | EILTGSWSDQTYPEGTQAIYKCRPGYRSLG | Complement factor H |
| | 223 | NVIMVCRKGEWVALNPLRKCQKRPCGHPGD | OS = Homo sapiens |
| | (8 May 2019) | TPFGTFTLTGGNVFEYGVKAVYTCNEGYQL | OX = 9606 GN = CFH |
| | Sequence | LGEINYRECDTDGWTNDIPICEVVKCLPVT | PE = 1 SV = 4 |
| | version 4 | APENGKIVSSAMEPDREYHFGQAVRFVCNS | |
| | (11 Sep. 2007) | GYKIEGDEEMHCSDDGFWSKEKPKCVEISC | |
| | | KSPDVINGSPISQKIIYKENERFQYKCNMG | |
| | | YEYSERGDAVCTESGWRPLPSCEEKSCDNP | |
| | | YIPNGDYSPLRIKHRTGDEITYQCRNGFYP | |
| | | ATRGNTAKCTSTGWIPAPRCTLKPCDYPDI | |
| | | KHGGLYHENMRRPYFPVAVGKYYSYYCDEH | |
| | | FETPSGSYWDHIHCTQDGWSPAVPCLRKCY | |
| | | FPYLENGYNQNYGRKFVQGKSIDVACHPGY | |
| | | ALPKAQTTVTCMENGWSPTPRCIRVKTCSK | |
| | | SSIDIENGFISESQYTYALKEKAKYQCKLG | |
| | | YVTADGETSGSITCGKDGWSAQPTCIKSCD | |
| | | IPVFMNARTKNDFTWFKLNDTLDYECHDGY | |
| | | ESNTGSTTGSIVCGYNGWSDLPICYERECE | |
| | | LPKIDVHLVPDRKKDQYKVGEVLKFSCKPG | |
| | | FTIVGPNSVQCYHFGLSPDLPICKEQVQSC | |
| | | GPPPELLNGNVKEKTKEEYGHSEVVEYYCN | |
| | | PRFLMKGPNKIQCVDGEWTTLPVCIVEEST | |
| | | CGDIPELEHGWAQLSSPPYYYGDSVEFNCS | |
| | | ESFTMIGHRSITCIHGVWTQLPQCVAIDKL | |
| | | KKCKSSNLIILEEHLKNKKEFDHNSNIRYR | |
| | | CRGKEGWIHTVCINGRWDPEVNCSMAQIQL | |
| | | CPPPPQIPNSHNMTTTLNYRDGEKVSVLCQ | |
| | | ENYLIQEGEEITCKDGRWQSIPLCVEKIPC | |
| | | SQPPQIEHGTINSSRSSQESYAHGTKLSYT | |
| | | CEGGFRISEENETTCYMGKWSSPPQCEGLP | |
| | | CKSPPEISHGVVAHMSDSYQYGEEVTYKCF | |
| | | EGFGIDGPAIAKCLGEKWSHPPSCIKTDCL | |
| | | SLPSFENAIPMGEKKDVYKAGEQVTYTCAT | |
| | | YYKMDGASNVTCINSRWTGRPTCRDTSCVN | |
| | | PPTVQNAYIVSRQMSKYPSGERVRYQCRSP | |
| | | YEMFGDEEVMCLNGNWTEPPQCKDSTGKCG | |
| | | PPPPIDNGDITSFPLSVYAPASSVEYQCQN | |
| | | LYQLEGNKRITCRNGQWSEPPKCLHPCVIS | |
| | | REIMENYNIALRWTAKQKLYSRTGESVEFV | |
| | | CKRGYRLSSRSHTLRTTCWDGKLEYPTCAK | |
| | | R | |
| 31 | P24001 | MCFPKVLSDDMKKLKARMVMLLPTSAQGLG | IL32_HUMAN |
| | Entry version | AWVSACDTEDTVGHLGPWRDKDPALWCQLC | Interleukin-32 |
| | 135 | LSSQHQAIERFYDKMQNAESGRGQVMSSLA | OS = Homo sapiens |
| | (8 May 2019) | ELEDDFKEGYLETVAAYYEEQHPELTPLLE | OX = 9606 GN = IL32 |
| | Sequence | KERDGLRCRGNRSPVPDVEDPATEEPGESF | PE = 1 SV = 3 |
| | version 3 | CDKVMRWFQAMLQRLQTWWHGVLAWVKEKV | |
| | (12 Dec. 2006) | VALVHAVQALWKQFQSFCCSLSELFMSSFQ | |
| | | SYGAPRGDKEELTPQKCSEPQSSK | |
| 32 | P04049 | MEHIQGAWKTISNGFGFKDAVFDGSSCISP | RAF1_HUMAN RAF |
| | Entry version | TIVQQFGYQRRASDDGKLTDPSKTSNTIRV | proto-oncogene |
| | 233 | FLPNKQRTVVNVRNGMSLHDCLMKALKVRG | serine/threonine- |
| | (5 Jun. 2019) | LQPECCAVFRLLHEHKGKKARLDWNTDAAS | protein kinase |
| | Sequence | LIGEELQVDFLDHVPLTTHNFARKTFLKLA | OS = Homo sapiens |
| | version 1 | FCDICQKFLLNGFRCQTCGYKFHEHCSTKV | OX = 9606 GN = RAF1 |
| | (1 Nov. 1986) | PTMCVDWSNIRQLLLFPNSTIGDSGVPALP | PE = 1 SV = 1 |
| | | SLTMRRMRESVSRMPVSSQHRYSTPHAFTF | |
| | | NTSSPSSEGSLSQRQRSTSTPNVHMVSTTL | |
| | | PVDSRMIEDAIRSHSESASPSALSSSPNNL | |
| | | SPTGWSQPKTPVPAQRERAPVSGTQEKNKI | |
| | | RPRGQRDSSYYWEIEASEVMLSTRIGSGSF | |
| | | GTVYKGKWHGDVAVKILKVVDPTPEQFQAF | |
| | | RNEVAVLRKTRHVNILLFMGYMTKDNLAIV | |
| | | TQWCEGSSLYKHLHVQETKFQMFQLIDIAR | |
| | | QTAQGMDYLHAKNIIHRDMKSNNIFLHEGL | |
| | | TVKIGDFGLATVKSRWSGSQQVEQPTGSVL | |
| | | WMAPEVIRMQDNNPFSFQSDVYSYGIVLYE | |
| | | LMTGELPYSHINNRDQIIFMVGRGYASPDL | |
| | | SKLYKNCPKAMKRLVADCVKKVKEERPLFP | |
| | | QILSSIELLQHSLPKINRSASEPSLHRAAH | |
| | | TEDINACTLTTSPRLPVF | |
| 33 | O94923 | MRCLAARVNYKTLIIICALFTLVTVLLWNK | GLCE_HUMAN D- |
| | Entry version | CSSDKAIQFPRRSSSGFRVDGFEKRAAASE | glucuronyl C5- |
| | 146 | SNNYMNHVAKQQSEEAFPQEQQKAPPVVGG | epimerase OS = Homo |
| | (5 Jun. 2019) | FNSNVGSKVLGLKYEEIDCLINDEHTIKGR | sapiens OX = 9606 |
| | Sequence | REGNEVFLPFTWVEKYFDVYGKVVQYDGYD | GN = GLCE PE = 1 |
| | version 3 | RFEFSHSYSKVYAQRAPYHPDGVFMSFEGY | SV = 3 |
| | (16 Jun. 2009) | NVEVRDRVKCISGVEGVPLSTQWGPQGYFY | |
| | | PIQIAQYGLSHYSKNLTEKPPHIEVYETAE | |
| | | DRDKNKPNDWTVPKGCFMANVADKSRFTNV | |
| | | KQFIAPETSEGVSLQLGNTKDFIISFDLKF | |
| | | LTNGSVSVVLETTEKNQLFTIHYVSNAQLI | |
| | | AFKERDIYYGIGPRTSWSTVTRDLVTDLRK | |
| | | GVGLSNTKAVKPTKIMPKKVVRLIAKGKGF | |
| | | LDNITISTTAHMAAFFAASDWLVRNQDEKG | |
| | | GWPIMVTRKLGEGFKSLEPGWYSAMAQGQA | |
| | | ISTLVRAYLLTKDHIFLNSALRATAPYKFL | |
| | | SEQHGVKAVFMNKHDWYEEYPTTPSSFVLN | |
| | | GFMYSLIGLYDLKETAGEKLGKEARSLYER | |
| | | GMESLKAMLPLYDTGSGTIYDLRHFMLGIA | |
| | | PNLARWDYHTTHINQLQLLSTIDESPVFKE | |
| | | FVKRWKSYLKGSRAKHN | |
| 34 | Q16186 | MTTSGALFPSLVPGSRGASNKYLVEFRAGK | ADRM1_HUMAN |
| | Entry version | MSLKGTTVTPDKRKGLVYIQQTDDSLIHFC | Proteasomal ubiquitin |
| | 174 | WKDRTSGNVEDDLIIFPDDCEFKRVPQCPS | receptor ADRM1 |
| | (5 Jun. 2019) | GRVYVLKFKAGSKRLFFWMQEPKTDQDEEH | OS = Homo sapiens |
| | Sequence | CRKVNEYLNNPPMPGALGASGSSGHELSAL | OX = 9606 |
| | version 2 | GGEGGLQSLLGNMSHSQLMQLIGPAGLGGL | GN = ADRM1 PE = 1 |
| | (23 Jan. 2002) | GGLGALTGPGLASLLGSSGPPGSSSSSSSR | SV = 2 |
| | | SQSAAVTPSSTTSSTRATPAPSAPAAASAT | |
| | | SPSPAPSSGNGASTAASPTQPIQLSDLQSI | |
| | | LATMNVPAGPAGGQQVDLASVLTPEIMAPI | |
| | | LANADVQERLLPYLPSGESLPQTADEIQNT | |
| | | LTSPQFQQALGMFSAALASGQLGPLMCQFG | |
| | | LPAEAVEAANKGDVEAFAKAMQNNAKPEQK | |
| | | EGDTKDKKDEEEDMSLD | |
| 35 | Q14995 | MEVNAGGVIAYISSSSSASSPASCHSEGSE | NR1D2_HUMAN |
| | Entry version | NSFQSSSSSVPSSPNSSNSDTNGNPKNGDL | Nuclear receptor |
| | 180 | ANIEGILKNDRIDCSMKTSKSSAPGMTKSH | subfamily 1 group D |
| | (5 Jun. 2019) | SGVTKFSGMVLLCKVCGDVASGFHYGVHAC | member 2 OS = Homo |
| | Sequence | EGCKGFFRRSIQQNIQYKKCLKNENCSIMR | sapiens OX = 9606 |
| | version 3 | MNRNRCQQCRFKKCLSVGMSRDAVRFGRIP | GN = NR1D2 PE = 1 |
| | (25 Nov. 2008) | KREKQRMLIEMQSAMKTMMNSQFSGHLQND | SV = 3 |
| | | TLVEHHEQTALPAQEQLRPKPQLEQENIKS | |
| | | SSPPSSDFAKEEVIGMVTRAHKDTFMYNQE | |
| | | QQENSAESMQPQRGERIPKNMEQYNLNHDH | |
| | | CGNGLSSHFPCSESQQHLNGQFKGRNIMHY | |
| | | PNGHAICIANGHCMNFSNAYTQRVCDRVPI | |
| | | DGFSQNENKNSYLCNTGGRMHLVCPLSKSP | |
| | | YVDPHKSGHEIWEEFSMSFTPAVKEVVEFA | |
| | | KRIPGFRDLSQHDQVNLLKAGTFEVLMVRF | |
| | | ASLFDAKERTVTFLSGKKYSVDDLHSMGAG | |
| | | DLLNSMFEFSEKLNALQLSDEEMSLFTAVV | |
| | | LVSADRSGIENVNSVEALQETLIRALRTLI | |
| | | MKNHPNEASIFTKLLLKLPDLRSLNNMHSE | |
| | | ELLAFKVHP | |
| 36 | P43320 | MASDHQTQAGKPQSLNPKIIIFEQENFQGH | CRBB2_HUMAN |
| | Entry version | SHELNGPCPNLKETGVEKAGSVLVQAGPWV | Beta-crystallin B2 |
| | 180 | GYEQANCKGEQFVFEKGEYPRWDSWTSSRR | OS = Homo sapiens |
| | (5 Jun. 2019) | TDSLSSLRPIKVDSQEHKIILYENPNFTGK | OX = 9606 |
| | Sequence | KMEIIDDDVPSFHAHGYQEKVSSVRVQSGT | GN = CRYBB2 PE = 1 |
| | version 2 | WVGYQYPGYRGLQYLLEKGDYKDSSDFGAP | SV = 2 |
| | (23 Jan. 2007) | HPQVQSVRRIRDMQWHQRGAFHPSN | |
| 37 | P51814 | MAANGDSPPWSPALAAEGRGSSCEVRRERT | ZNF41_HUMAN Zinc |
| | Entry version | PEARIHSVKRYPDLSPGPKGRSSADHAALN | finger protein 41 |
| | 193 | SIVSLQASVSFEDVTVDFSKEEWQHLDPAQ | OS = Homo sapiens |
| | (8 May 2019) | RRLYWDVTLENYSHLLSVGYQIPKSEAAFK | OX = 9606 GN = ZNF41 |
| | Sequence | LEQGEGPWMLEGEAPHQSCSGEAIGKMQQQ | PE = 1 SV = 2 |
| | version 2 | GIPGGIFFHCERFDQPIGEDSLCSILEELW | |
| | (23 Jan. 2002) | QDNDQLEQRQENQNNLLSHVKVLIKERGYE | |
| | | HKNIEKIIHVTTKLVPSIKRLHNCDTILKH | |
| | | TLNSHNHNRNSATKNLGKIFGNGNNFPHSP | |
| | | SSTKNENAKTGANSCEHDHYEKHLSHKQAP | |
| | | THHQKIHPEEKLYVCTECVMGFTQKSHLFE | |
| | | HQRIHAGEKSRECDKSNKVFPQKPQVDVHP | |
| | | SVYTGEKPYLCTQCGKVFTLKSNLITHQKI | |
| | | HTGQKPYKCSECGKAFFQRSDLFRHLRIHT | |
| | | GEKPYECSECGKGFSQNSDLSIHQKTHTGE | |
| | | KHYECNECGKAFTRKSALRMHQRIHTGEKP | |
| | | YVCADCGKAFIQKSHFNTHQRIHTGEKPYE | |
| | | CSDCGKSFTKKSQLHVHQRIHTGEKPYICT | |
| | | ECGKVFTHRTNLTTHQKTHTGEKPYMCAEC | |
| | | GKAFTDQSNLIKHQKTHTGEKPYKCNGCGK | |
| | | AFIWKSRLKIHQKSHIGERHYECKDCGKAF | |
| | | IQKSTLSVHQRIHTGEKPYVCPECGKAFIQ | |
| | | KSHFIAHHRIHTGEKPYECSDCGKCFTKKS | |
| | | QLRVHQKIHTGEKPNICAECGKAFTDRSNL | |
| | | ITHQKIHTREKPYECGDCGKTFTWKSRLNI | |
| | | HQKSHTGERHYECSKCGKAFIQKATLSMHQ | |
| | | IIHTGKKPYACTECQKAFTDRSNLIKHQKM | |
| | | HSGEKRYKASD | |
| 38 | P19419 | MDPSVTLWQFLLQLLREQGNGHIISWTSRD | ELK1_HUMAN ETS |
| | Entry version | GGEFKLVDAEEVARLWGLRKNKTNMNYDKL | domain-containing |
| | 210 | SRALRYYYDKNIIRKVSGQKFVYKFVSYPE | protein Elk-1 |
| | (8 May 2019) | VAGCSTEDCPPQPEVSVTSTMPNVAPAAIH | OS = Homo sapiens |
| | Sequence | AAPGDTVSGKPGTPKGAGMAGPGGLARSSR | OX = 9606 GN = ELK1 |
| | version 2 | NEYMRSGLYSTFTIQSLQPQPPPHPRPAVV | PE = 1 SV = 2 |
| | (24 Jan. 2001) | LPSAAPAGAAAPPSGSRSTSPSPLEACLEA | |
| | | EEAGLPLQVILTPPEAPNLKSEELNVEPGL | |
| | | GRALPPEVKVEGPKEELEVAGERGFVPETT | |
| | | KAEPEVPPQEGVPARLPAVVMDTAGQAGGH | |
| | | AASSPEISQPQKGRKPRDLELPLSPSLLGG | |
| | | PGPERTPGSGSGSGLQAPGPALTPSLLPTH | |
| | | TLTPVLLTPSSLPPSIHFWSTLSPIAPRSP | |
| | | AKLSFQFPSSGSAQVHIPSISVDGLSTPVV | |
| | | LSPGPQKP | |
| 39 | P43080 | MGNVMEGKSVEELSSTECHQWYKKFMTECP | GUC1A_HUMAN |
| | Entry version | SGQLTLYEFRQFFGLKNLSPSASQYVEQMF | Guanylyl cyclase- |
| | 186 | ETFDFNKDGYIDFMEYVAALSLVLKGKVEQ | activating protein 1 |
| | (8 May 2019) | KLRWYFKLYDVDGNGCIDRDELLTIIQAIR | OS = Homo sapiens |
| | Sequence | AINPCSDTTMTAEEFTDTVFSKIDVNGDGE | OX = 9606 |
| | version 3 | LSLEEFIEGVQKDQMLLDTLTRSLDLTRIV | GN = GUCA1A PE = 1 |
| | (23 Jan. 2007) | RRLQNGEQDEEGADEAAEAAG | SV = 3 |
| 40 | P10914 | MPITRMRMRPWLEMQINSNQIPGLIWINKE | IRF1_HUMAN |
| | Entry version | EMIFQIPWKHAAKHGWDINKDACLFRSWAI | Interferon regulatory |
| | 179 | HTGRYKAGEKEPDPKTWKANFRCAMNSLPD | factor 1 OS = Homo |
| | (8 May 2019) | IEEVKDQSRNKGSSAVRVYRMLPPLTKNQR | sapiens OX = 9606 |
| | Sequence | KERKSKSSRDAKSKAKRKSCGDSSPDTFSD | GN = IRF1 PE = 1 SV = 2 |
| | version 2 | GLSSSTLPDDHSSYTVPGYMQDLEVEQALT | |
| | (16 Apr. 2002) | PALSPCAVSSTLPDWHIPVEVVPDSTSDLY | |
| | | NFQVSPMPSTSEATTDEDEEGKLPEDIMKL | |
| | | LEQSEWQPTNVDGKGYLLNEPGVQPTSVYG | |
| | | DFSCKEEPEIDSPGGDIGLSLQRVFTDLKN | |
| | | MDATWLDSLLTPVRLPSIQAIPCAP | |
| 41 | Q14457 | MEGSKTSNNSTMQVSFVCQRCSQPLKLDTS | BECN1_HUMAN |
| | Entry version | FKILDRVTIQELTAPLLTTAQAKPGETQEE | Beclin-1 OS = Homo |
| | 189 | ETNSGEEPFIETPRQDGVSRRFIPPARMMS | sapiens OX = 9606 |
| | (5 Jun. 2019) | TESANSFTLIGEASDGGTMENLSRRLKVTG | GN = BECN1 PE = 1 |
| | Sequence | DLFDIMSGQTDVDHPLCEECTDTLLDQLDT | SV = 2 |
| | version 2 | QLNVTENECQNYKRCLEILEQMNEDDSEQL | |
| | (21 Feb. 2001) | QMELKELALEEERLIQELEDVEKNRKIVAE | |
| | | NLEKVQAEAERLDQEEAQYQREYSEFKRQQ | |
| | | LELDDELKSVENQMRYAQTQLDKLKKTNVF | |
| | | NATFHIWHSGQFGTINNFRLGRLPSVPVEW | |
| | | NEINAAWGQTVLLLHALANKMGLKFQRYRL | |
| | | VPYGNHSYLESLTDKSKELPLYCSGGLRFF | |
| | | WDNKFDHAMVAFLDCVQQFKEEVEKGETRF | |
| | | CLPYRMDVEKGKIEDTGGSGGSYSIKTQFN | |
| | | SEEQWTKALKFMLTNLKWGLAWVSSQFYNK | |
| 42 | Q01968 | MEPPLPVGAQPLATVEGMEMKGPLREPCAL | OCRL_HUMAN |
| | Entry version | TLAQRNGQYELIIQLHEKEQHVQDIIPINS | Inositol |
| | 205 | HFRCVQEAEETLLIDIASNSGCKIRVQGDW | polyphosphate 5- |
| | (8 May 2019) | IRERRFEIPDEEHCLKFLSAVLAAQKAQSQ | phosphatase OCRL-1 |
| | Sequence | LLVPEQKDSSSWYQKLDTKDKPSVFSGLLG | OS = Homo sapiens |
| | version 3 | FEDNFSSMNLDKKINSQNQPTGIHREPPPP | OX = 9606 GN = OCRL |
| | (7 Jun. 2005) | PFSVNKMLPREKEASNKEQPKVTNTMRKLF | PE = 1 SV = 3 |
| | | VPNTQSGQREGLIKHILAKREKEYVNIQTF | |
| | | RFFVGTWNVNGQSPDSGLEPWLNCDPNPPD | |
| | | IYCIGFQELDLSTEAFFYFESVKEQEWSMA | |
| | | VERGLHSKAKYKKVQLVRLVGMMLLIFARK | |
| | | DQCRYIRDIATETVGTGIMGKMGNKGGVAV | |
| | | RFVFHNTTFCIVNSHLAAHVEDFERRNQDY | |
| | | KDICARMSFVVPNQTLPQLNIMKHEVVIWL | |
| | | GDLNYRLCMPDANEVKSLINKKDLQRLLKF | |
| | | DQLNIQRTQKKAFVDFNEGEIKFIPTYKYD | |
| | | SKTDRWDSSGKCRVPAWCDRILWRGTNVNQ | |
| | | LNYRSHMELKTSDHKPVSALFHIGVKVVDE | |
| | | RRYRKVFEDSVRIMDRMENDFLPSLELSRR | |
| | | EFVFENVKFRQLQKEKFQISNNGQVPCHFS | |
| | | FIPKLNDSQYCKPWLRAEPFEGYLEPNETV | |
| | | DISLDVYVSKDSVTILNSGEDKIEDILVLH | |
| | | LDRGKDYFLTISGNYLPSCFGTSLEALCRM | |
| | | KRPIREVPVTKLIDLEEDSFLEKEKSLLQM | |
| | | VPLDEGASERPLQVPKEIWLLVDHLFKYAC | |
| | | HQEDLFQTPGMQEELQQIIDCLDTSIPETI | |
| | | PGSNHSVAEALLIFLEALPEPVICYELYQR | |
| | | CLDSAYDPRICRQVISQLPRCHRNVFRYLM | |
| | | AFLRELLKFSEYNSVNANMIATLFTSLLLR | |
| | | PPPNLMARQTPSDRQRAIQFLLGFLLGSEE | |
| | | D | |
| 43 | P63172 | MEDYQAAEETAFVVDEVSNIVKEAIESAIG | DYLT1_HUMAN |
| | Entry version | GNAYQHSKVNQWTTNVVEQTLSQLTKLGKP | Dynein light chain |
| | 137 | FKYIVTCVIMQKNGAGLHTASSCFWDSSTD | Tctex-type 1 |
| | (8 May 2019) | GSCTVRWENKTMYCIVSAFGLSI | OS = Homo sapiens |
| | Sequence | | OX = 9606 |
| | version 1 | | GN = DYNLT1 PE = 1 |
| | (27 Sep. 2004) | | SV = 1 |
| 44 | P35442 | MVWRLVLLALWVWPSTQAGHQDKDTTFDLF | TSP2_HUMAN |
| | Entry version | SISNINRKTIGAKQFRGPDPGVPAYRFVRF | Thrombospondin-2 |
| | 192 | DYIPPVNADDLSKITKIMRQKEGFFLTAQL | OS = Homo sapiens |
| | (5 Jun. 2019) | KQDGKSRGTLLALEGPGLSQRQFEIVSNGP | OX = 9606 |
| | Sequence | ADTLDLTYWIDGTRHVVSLEDVGLADSQWK | GN = THBS2 PE = 1 |
| | version 2 | NVTVQVAGETYSLHVGCDLIDSFALDEPFY | SV = 2 |
| | (25 Nov. 2008) | EHLQAEKSRMYVAKGSARESHFRGLLQNVH | |
| | | LVFENSVEDILSKKGCQQGQGAEINAISEN | |
| | | TETLRLGPHVTTEYVGPSSERRPEVCERSC | |
| | | EELGNMVQELSGLHVLVNQLSENLKRVSND | |
| | | NQFLWELIGGPPKTRNMSACWQDGRFFAEN | |
| | | ETWVVDSCTTCTCKKFKTICHQITCPPATC | |
| | | ASPSFVEGECCPSCLHSVDGEEGWSPWAEW | |
| | | TQCSVTCGSGTQQRGRSCDVTSNTCLGPSI | |
| | | QTRACSLSKCDTRIRQDGGWSHWSPWSSCS | |
| | | VTCGVGNITRIRLCNSPVPQMGGKNCKGSG | |
| | | RETKACQGAPCPIDGRWSPWSPWSACTVTC | |
| | | AGGIRERTRVCNSPEPQYGGKACVGDVQER | |
| | | QMCNKRSCPVDGCLSNPCFPGAQCSSFPDG | |
| | | SWSCGSCPVGFLGNGTHCEDLDECALVPDI | |
| | | CFSTSKVPRCVNTQPGFHCLPCPPRYRGNQ | |
| | | PVGVGLEAAKTEKQVCEPENPCKDKTHNCH | |
| | | KHAECIYLGHFSDPMYKCECQTGYAGDGLI | |
| | | CGEDSDLDGWPNLNLVCATNATYHCIKDNC | |
| | | PHLPNSGQEDFDKDGIGDACDDDDDNDGVT | |
| | | DEKDNCQLLFNPRQADYDKDEVGDRCDNCP | |
| | | YVHNPAQIDTDNNGEGDACSVDIDGDDVFN | |
| | | ERDNCPYVYNTDQRDTDGDGVGDHCDNCPL | |
| | | VHNPDQTDVDNDLVGDQCDNNEDIDDDGHQ | |
| | | NNQDNCPYISNANQADHDRDGQGDACDPDD | |
| | | DNDGVPDDRDNCRLVFNPDQEDLDGDGRGD | |
| | | ICKDDFDNDNIPDIDDVCPENNAISETDFR | |
| | | NFQMVPLDPKGTTQIDPNWVIRHQGKELVQ | |
| | | TANSDPGIAVGFDEFGSVDFSGTFYVNTDR | |
| | | DDDYAGFVFGYQSSSRFYVVMWKQVTQTYW | |
| | | EDQPTRAYGYSGVSLKVVNSTTGTGEHLRN | |
| | | ALWHTGNTPGQVRTLWHDPRNIGWKDYTAY | |
| | | RWHLTHRPKTGYIRVLVHEGKQVMADSGPI | |
| | | YDQTYAGGRLGLFVFSQEMVYFSDLKYECR | |
| | | DI | |
| 45 | Q92583 | MAPLKMLALVTLLLGASLQHIHAARGTNVG | CCL17_HUMAN C-C |
| | Entry version | RECCLEYFKGAIPLRKLKTWYQTSEDCSRD | motif chemokine 17 |
| | 165 | AIVFVTVQGRAICSDPNNKRVKNAVKYLQS | OS = Homo sapiens |
| | (5 Jun. 2019) | LERS | OX = 9606 GN = CCL17 |
| | Sequence | | PE = 1 SV = 1 |
| | version 1 | | |
| | (1 Feb. 1997) | | |
| 46 | A6NNA5 | MFYFHCPPQLEGTATFGNHSSGDFDDGFLR | DRGX_HUMAN |
| | Entry version | RKQRRNRTTFTLQQLEALEAVFAQTHYPDV | Dorsal root ganglia |
| | 92 | FTREELAMKINLTEARVQVWFQNRRAKWRK | homeobox protein |
| | (8 May 2019) | TERGASDQEPGAKEPMAEVTPPPVRNINSP | OS = Homo sapiens |
| | Sequence | PPGDQARSKKEALEAQQSLGRTVGPAGPFF | OX = 9606 GN = DRGX |
| | version 1 | PSCLPGTLLNTATYAQALSHVASLKGGPLC | PE = 3 SV = 1 |
| | (24 Jul. 2007) | SCCVPDPMGLSFLPTYGCQSNRTASVATLR | |
| | | MKAREHSEAVLQSANLLPSTSSSPGPVAKP | |
| | | APPDGSQEKTSPTKEQSEAEKSV | |
| 47 | P01210 | MARFLTLCTWLLLLGPGLLATVRAECSQDC | PENK_HUMAN |
| | Entry version | ATCSYRLVRPADINFLACVMECEGKLPSLK | Proenkephalin-A |
| | 168 | IWETCKELLQLSKPELPQDGTSTLRENSKP | OS = Homo sapiens |
| | (8 May 2019) | EESHLLAKRYGGFMKRYGGFMKKMDELYPM | OX = 9606 GN = PENK |
| | Sequence | EPEEEANGSEILAKRYGGFMKKDAEEDDSL | PE = 1 SV = 1 |
| | version 1 | ANSSDLLKELLETGDNRERSHHQDGSDNEE | |
| | (21 Jul. 1986) | EVSKRYGGFMRGLKRSPQLEDEAKELQKRY | |
| | | GGFMRRVGRPEWWMDYQKRYGGFLKRFAEA | |
| | | LPSDEEGESYSKEVPEMEKRYGGFMRF | |
| 48 | Q9HBG7 | MVAPKSHTDDWAPGPFSSKPQRSQLQIFSS | LY9_HUMAN T- |
| | Entry version | VLQTSLLFLLMGLRASGKDSAPTVVSGILG | lymphocyte surface |
| | 149 | GSVTLPLNISVDTEIENVIWIGPKNALAFA | antigen Ly-9 |
| | (8 May 2019) | RPKENVTIMVKSYLGRLDITKWSYSLCISN | OS = Homo sapiens |
| | Sequence | LTLNDAGSYKAQINQRNFEVTTEEEFTLFV | OX = 9606 GN = LY9 |
| | version 3 | YEQLQEPQVTMKSVKVSENFSCNITLMCSV | PE = 1 SV = 3 |
| | (19 Jul. 2005) | KGAEKSVLYSWTPREPHASESNGGSILTVS | |
| | | RTPCDPDLPYICTAQNPVSQRSSLPVHVGQ | |
| | | FCTDPGASRGGTTGETVVGVLGEPVTLPLA | |
| | | LPACRDTEKVVWLFNTSIISKEREEAATAD | |
| | | PLIKSRDPYKNRVWVSSQDCSLKISQLKIE | |
| | | DAGPYHAYVCSEASSVTSMTHVTLLIYRRL | |
| | | RKPKITWSLRHSEDGICRISLTCSVEDGGN | |
| | | TVMYTWTPLQKEAVVSQGESHLNVSWRSSE | |
| | | NHPNLTCTASNPVSRSSHQFLSENICSGPE | |
| | | RNTKLWIGLFLMVCLLCVGIFSWCIWKRKG | |
| | | RCSVPAFCSSQAEAPADTPEPTAGHTLYSV | |
| | | LSQGYEKLDTPLRPARQQPTPTSDSSSDSN | |
| | | LTTEEDEDRPEVHKPISGRYEVFDQVTQEG | |
| | | AGHDPAPEGQADYDPVTPYVTEVESVVGEN | |
| | | TMYAQVFNLQGKTPVSQKEESSATIYCSIR | |
| | | KPQVVPPPQQNDLEIPESPTYENFT | |
| 49 | O15146 | MRELVNIPLVHILTLVAFSGTEKLPKAPVI | MUSK_HUMAN |
| | Entry version | TTPLETVDALVEEVATFMCAVESYPQPEIS | Muscle, skeletal |
| | 169 | WTRNKILIKLFDTRYSIRENGQLLTILSVE | receptor tyrosine- |
| | (8 May 2019) | DSDDGIYCCTANNGVGGAVESCGALQVKMK | protein kinase |
| | Sequence | PKITRPPINVKIIEGLKAVLPCTTMGNPKP | OS = Homo sapiens |
| | version 1 | SVSWIKGDSPLRENSRIAVLESGSLRIHNV | OX = 9606 GN = MUSK |
| | (1 Jan. 1998) | QKEDAGQYRCVAKNSLGTAYSKVVKLEVEV | PE = 1 SV = 1 |
| | | FARILRAPESHNVTFGSFVTLHCTATGIPV | |
| | | PTITWIENGNAVSSGSIQESVKDRVIDSRL | |
| | | QLFITKPGLYTCIATNKHGEKFSTAKAAAT | |
| | | ISIAEWSKPQKDNKGYCAQYRGEVCNAVLA | |
| | | KDALVFLNTSYADPEEAQELLVHTAWNELK | |
| | | VVSPVCRPAAEALLCNHIFQECSPGVVPTP | |
| | | IPICREYCLAVKELFCAKEWLVMEEKTHRG | |
| | | LYRSEMHLLSVPECSKLPSMHWDPTACARL | |
| | | PHLDYNKENLKTFPPMTSSKPSVDIPNLPS | |
| | | SSSSSFSVSPTYSMTVIISIMSSFAIFVLL | |
| | | TITTLYCCRRRKQWKNKKRESAAVTLTTLP | |
| | | SELLLDRLHPNPMYQRMPLLLNPKLLSLEY | |
| | | PRNNIEYVRDIGEGAFGRVFQARAPGLLPY | |
| | | EPFTMVAVKMLKEEASADMQADFQREAALM | |
| | | AEFDNPNIVKLLGVCAVGKPMCLLFEYMAY | |
| | | GDLNEFLRSMSPHTVCSLSHSDLSMRAQVS | |
| | | SPGPPPLSCAEQLCIARQVAAGMAYLSERK | |
| | | FVHRDLATRNCLVGENMVVKIADFGLSRNI | |
| | | YSADYYKANENDAIPIRWMPPESIFYNRYT | |
| | | TESDVWAYGVVLWEIFSYGLQPYYGMAHEE | |
| | | VIYYVRDGNILSCPENCPVELYNLMRLCWS | |
| | | KLPADRPSFTSIHRILERMCERAEGTVSV | |
| 50 | P28698 | MRPAVLGSPDRAPPEDEGPVMVKLEDSEEE | MZF1_HUMAN |
| | Entry version | GEAALWDPGPEAARLRFRCFRYEEATGPQE | Myeloid zinc finger 1 |
| | 194 | ALAQLRELCRQWLRPEVRSKEQMLELLVLE | OS = Homo sapiens |
| | (8 May 2019) | QFLGALPPEIQARVQGQRPGSPEEAAALVD | OX = 9606 GN = MZF1 |
| | Sequence | GLRREPGGPRRWVTVQVQGQEVLSEKMEPS | PE = 1 SV = 3 |
| | version 3 | SFQPLPETEPPTPEPGPKTPPRTMQESPLG | |
| | (25 Nov. 2008) | LQVKEESEVTEDSDFLESGPLAATQESVPT | |
| | | LLPEEAQRCGTVLDQIFPHSKTGPEGPSWR | |
| | | EHPRALWHEEAGGIFSPGFALQLGSISAGP | |
| | | GSVSPHLHVPWDLGMAGLSGQIQSPSREGG | |
| | | FAHALLLPSDLRSEQDPTDEDPCRGVGPAL | |
| | | ITTRWRSPRGRSRGRPSTGGGVVRGGRCDV | |
| | | CGKVFSQRSNLLRHQKIHTGERPFVCSECG | |
| | | RSFSRSSHLLRHQLTHTEERPFVCGDCGQG | |
| | | FVRSARLEEHRRVHTGEQPFRCAECGQSFR | |
| | | QRSNLLQHQRIHGDPPGPGAKPPAPPGAPE | |
| | | PPGPFPCSECRESFARRAVLLEHQAVHTGD | |
| | | KSFGCVECGERFGRRSVLLQHRRVHSGERP | |
| | | FACAECGQSFRQRSNLTQHRRIHTGERPFA | |
| | | CAECGKAFRQRPTLTQHLRVHTGEKPFACP | |
| | | ECGQRFSQRLKLTRHQRTHTGEKPYHCGEC | |
| | | GLGFTQVSRLTEHQRIHTGERPFACPECGQ | |
| | | SFRQHANLTQHRRIHTGERPYACPECGKAF | |
| | | RQRPTLTQHLRTHRREKPFACQDCGRRFHQ | |
| | | STKLIQHQRVHSAE | |
| 51 | Q14129 | MERYAGALEEVADGARQQERHYQLLSALQS | DGCR6_HUMAN |
| | Entry version | LVKELPSSFQQRLSYTTLSDLALALLDGTV | Protein DGCR6 |
| | 141 | FEIVQGLLEIQHLTEKSLYNQRLRLQNEHR | OS = Homo sapiens |
| | (5 Jun. 2019) | VLRQALRQKHQEAQQACRPHNLPVLQAAQQ | OX = 9606 |
| | Sequence | RELEAVEHRIREEQRAMDQKIVLELDRKVA | GN = DGCR6 PE = 1 |
| | version 3 | DQQSTLEKAGVAGFYVTTNPQELMLQMNLL | SV = 3 |
| | (26 Jul. 2002) | ELIRKLQQRGCWAGKAALGLGGPWQLPAAQ | |
| | | CDQKGSPVPP | |
| 52 | Q9UNE0 | MAHVGDCTQTPWLPVLVVSLMCSARAEYSN | EDAR_HUMAN |
| | Entry version | CGENEYYNQTTGLCQECPPCGPGEEPYLSC | Tumor necrosis factor |
| | 158 | GYGTKDEDYGCVPCPAEKFSKGGYQICRRH | receptor superfamily |
| | (8 May 2019) | KDCEGFFRATVLTPGDMENDAECGPCLPGY | member EDAR |
| | Sequence | YMLENRPRNIYGMVCYSCLLAPPNTKECVG | OS = Homo sapiens |
| | version 1 | ATSGASANFPGTSGSSTLSPFQHAHKELSG | OX = 9606 GN = EDAR |
| | (1 May 2000) | QGHLATALIIAMSTIFIMAIAIVLIIMFYI | PE = 1 SV = 1 |
| | | LKTKPSAPACCTSHPGKSVEAQVSKDEEKK | |
| | | EAPDNVVMFSEKDEFEKLTATPAKPTKSEN | |
| | | DASSENEQLLSRSVDSDEEPAPDKQGSPEL | |
| | | CLLSLVHLAREKSATSNKSAGIQSRRKKIL | |
| | | DVYANVCGVVEGLSPTELPFDCLEKTSRML | |
| | | SSTYNSEKAVVKTWRHLAESFGLKRDEIGG | |
| | | MTDGMQLFDRISTAGYSIPELLTKLVQIER | |
| | | LDAVESLCADILEWAGVVPPASQPHAAS | |
| 53 | Q9UN72 | MVCPNGYDPGGRHLLLFIIILAAWEAGRGQ | PCDA7_HUMAN |
| | Entry version | LHYSVPEEAKHGNFVGRIAQDLGLELAELV | Protocadherin alpha- |
| | 155 | PRLFRAVCKFRGDLLEVNLQNGILFVNSRI | 7 OS = Homo sapiens |
| | (8 May 2019) | DREELCGRSAECSIHLEVIVERPLQVFHVD | OX = 9606 |
| | Sequence | VEVKDINDNPPVFPATQRNLFIAESRPLDS | GN = PCDHA7 PE = 2 |
| | version 1 | RFPLEGASDADIGENALLTYRLSPNEYFFL | SV = 1 |
| | (1 May 2000) | DVPTSNQQVKPLGLVLRKLLDREETPELHL | |
| | | LLTATDGGKPELTGTVQLLITVLDNNDNAP | |
| | | VFDRTLYTVKLPENVSIGTLVIHPNASDLD | |
| | | EGLNGDIIYSFSSDVSPDIKSKFHMDPLSG | |
| | | AITVIGHMDFEESRAHKIPVEAVDKGFPPL | |
| | | AGHCTVLVEVVDVNDNAPQLTLTSLSLPIP | |
| | | EDAQPGTVITLISVFDRDFGVNGQVTCSLT | |
| | | PRVPFKLVSTFKNYYSLVLDSALDRESVSA | |
| | | YELVVTARDGGSPSLWATASVSVEVADVND | |
| | | NAPAFAQPEYTVFVKENNPPGCHIFTVSAG | |
| | | DADAQKNALVSYSLVELRVGERALSSYVSV | |
| | | HAESGKVYALQPLDHEELELLQFQVSARDA | |
| | | GVPPLGSNVTLQVFVLDENDNAPALLAPRV | |
| | | GGTGGAVRELVPRSVGAGHVVAKVRAVDAD | |
| | | SGYNAWLSYELQPVAAGASIPFRVGLYTGE | |
| | | ISTTRALDETDAPRHRLLVLVKDHGEPSLT | |
| | | ATATVLVSLVESGQAPKASSRASLGIAGPE | |
| | | TELVDVNVYLIIAICAVSSLLVLTLLLYTA | |
| | | LRCSAPSSEGACSLVKPTLVCSSAVGSWSF | |
| | | SQQRRQRVCSGEGPPKTDLMAFSPSLPQGP | |
| | | SSTDNPRQPNPDWRYSASLRAGMHSSVHLE | |
| | | EAGILRAGPGGPDQQWPTVSSATPEPEAGE | |
| | | VSPPVGAGVNSNSWTFKYGPGNPKQSGPGE | |
| | | LPDKFIIPGSPAIISIRQEPTNSQIDKSDF | |
| | | ITFGKKEETKKKKKKKKGNKTQEKKEKGNS | |
| | | TTDNSDQ | |
| 54 | P12314 | MWFLTTLLLWVPVDGQVDTTKAVITLQPPW | FCGR1_HUMAN |
| | Entry version | VSVFQEETVTLHCEVLHLPGSSSTQWFLNG | High affinity |
| | 195 | TATQTSTPSYRITSASVNDSGEYRCQRGLS | immunoglobulin |
| | (5 Jun. 2019) | GRSDPIQLEIHRGWLLLQVSSRVFTEGEPL | gamma Fc receptor I |
| | Sequence | ALRCHAWKDKLVYNVLYYRNGKAFKFFHWN | OS = Homo sapiens |
| | version 2 | SNLTILKTNISHNGTYHCSGMGKHRYTSAG | OX = 9606 |
| | (19 Jul. 2004) | ISVTVKELFPAPVLNASVTSPLLEGNLVTL | GN = FCGR1A PE = 1 |
| | | SCETKLLLQRPGLQLYFSFYMGSKTLRGRN | SV = 2 |
| | | TSSEYQILTARREDSGLYWCEAATEDGNVL | |
| | | KRSPELELQVLGLQLPTPVWFHVLFYLAVG | |
| | | IMFLVNTVLWVTIRKELKRKKKWDLEISLD | |
| | | SGHEKKVISSLQEDRHLEEELKCQEQKEEQ | |
| | | LQEGVHRKEPQGAT | |
| 55 | P29965 | MIETYNQTSPRSAATGLPISMKIFMYLLTV | CD40L_HUMAN |
| | Entry version | FLITQMIGSALFAVYLHRRLDKIEDERNLH | CD40 ligand |
| | 208 | EDFVFMKTIQRCNTGERSLSLLNCEEIKSQ | OS = Homo sapiens |
| | (8 May 2019) | FEGFVKDIMLNKEETKKENSFEMQKGDQNP | OX = 9606 |
| | Sequence | QIAAHVISEASSKTTSVLQWAEKGYYTMSN | GN = CD40LG PE = 1 |
| | version 1 | NLVTLENGKQLTVKRQGLYYIYAQVTFCSN | SV = 1 |
| | (1 Apr. 1993) | REASSQAPFIASLCLKSPGRFERILLRAAN | |
| | | THSSAKPCGQQSIHLGGVFELQPGASVFVN | |
| | | VTDPSQVSHGTGFTSFGLLKL | |
| 56 | Q9UBU2 | MAALMRSKDSSCCLLLLAAVLMVESSQIGS | DKK2_HUMAN |
| | Entry version | SRAKLNSIKSSLGGETPGQAANRSAGMYQG | Dickkopf-related |
| | 148 | LAFGGSKKGKNLGQAYPCSSDKECEVGRYC | protein 2 OS = Homo |
| | (8 May 2019) | HSPHQGSSACMVCRRKKKRCHRDGMCCPST | sapiens OX = 9606 |
| | Sequence | RCNNGICIPVTESILTPHIPALDGTRHRDR | GN = DKK2 PE = 2 |
| | version 1 | NHGHYSNHDLGWQNLGRPHTKMSHIKGHEG | SV = 1 |
| | (1 May 2000) | DPCLRSSDCIEGFCCARHFWTKICKPVLHQ | |
| | | GEVCTKQRKKGSHGLEIFQRCDCAKGLSCK | |
| | | VWKDATYSSKARLHVCQKI | |
| 57 | P09341 | MARAALSAAPSNPRLLRVALLLLLLVAAGR | GROA_HUMAN |
| | Entry version | RAAGASVATELRCQCLQTLQGIHPKNIQSV | Growth-regulated |
| | 193 | NVKSPGPHCAQTEVIATLKNGRKACLNPAS | alpha protein |
| | (5 Jun. 2019) | PIVKKIIEKMLNSDKSN | OS = Homo sapiens |
| | Sequence | | OX = 9606 |
| | version 1 | | GN = CXCL1 PE = 1 |
| | (1 Jul. 1989) | | SV = 1 |
| 58 | P16035 | MGAAARTLRLALGLLLLATLLRPADACSCS | TIMP2_HUMAN |
| | Entry version | PVHPQQAFCNADVVIRAKAVSEKEVDSGND | Metalloproteinase |
| | 196 | IYGNPIKRIQYEIKQIKMFKGPEKDIEFIY | inhibitor 2 OS = Homo |
| | (8 May 2019) | TAPSSAVCGVSLDVGGKKEYLIAGKAEGDG | sapiens OX = 9606 |
| | Sequence | KMHITLCDFIVPWDTLSTTQKKSLNHRYQM | GN = TIMP2 PE = 1 |
| | version 2 | GCECKITRCPMIPCYISSPDECLWMDWVTE | SV = 2 |
| | (1 Nov. 1990) | KNINGHQAKFFACIKRSDGSCAWYRGAAPP | |
| | | KQEFLDIEDP | |
| 59 | Q9BWV1 | MLRGTMTAWRGMRPEVTLACLLLATAGCFA | BOC_HUMAN |
| | Entry version | DLNEVPQVTVQPASTVQKPGGTVILGCVVE | Brother of CDO |
| | 149 | PPRMNVTWRLNGKELNGSDDALGVLITHGT | OS = Homo sapiens |
| | (8 May 2019) | LVITALNNHTVGRYQCVARMPAGAVASVPA | OX = 9606 GN = BOC |
| | Sequence | TVTLANLQDFKLDVQHVIEVDEGNTAVIAC | PE = 1 SV = 1 |
| | version 1 | HLPESHPKAQVRYSVKQEWLEASRGNYLIM | |
| | (1 Jun. 2001) | PSGNLQIVNASQEDEGMYKCAAYNPVTQEV | |
| | | KTSGSSDRLRVRRSTAEAARIIYPPEAQTI | |
| | | IVTKGQSLILECVASGIPPPRVTWAKDGSS | |
| | | VTGYNKTRFLLSNLLIDTTSEEDSGTYRCM | |
| | | ADNGVGQPGAAVILYNVQVFEPPEVTMELS | |
| | | QLVIPWGQSAKLTCEVRGNPPPSVLWLRNA | |
| | | VPLISSQRLRLSRRALRVLSMGPEDEGVYQ | |
| | | CMAENEVGSAHAVVQLRTSRPSITPRLWQD | |
| | | AELATGTPPVSPSKLGNPEQMLRGQPALPR | |
| | | PPTSVGPASPQCPGEKGQGAPAEAPIILSS | |
| | | PRTSKTDSYELVWRPRHEGSGRAPILYYVV | |
| | | KHRKVTNSSDDWTISGIPANQHRLTLTRLD | |
| | | PGSLYEVEMAAYNCAGEGQTAMVTFRTGRR | |
| | | PKPEIMASKEQQIQRDDPGASPQSSSQPDH | |
| | | GRLSPPEAPDRPTISTASETSVYVTWIPRG | |
| | | NGGFPIQSFRVEYKKLKKVGDWILATSAIP | |
| | | PSRLSVEITGLEKGTSYKFRVRALNMLGES | |
| | | EPSAPSRPYVVSGYSGRVYERPVAGPYITF | |
| | | TDAVNETTIMLKWMYIPASNNNTPIHGFYI | |
| | | YYRPTDSDNDSDYKKDMVEGDKYWHSISHL | |
| | | QPETSYDIKMQCFNEGGESEFSNVMICETK | |
| | | ARKSSGQPGRLPPPTLAPPQPPLPETIERP | |
| | | VGTGAMVARSSDLPYLIVGVVLGSIVLIIV | |
| | | TFIPFCLWRAWSKQKHTTDLGFPRSALPPS | |
| | | CPYTMVPLGGLPGHQASGQPYLSGISGRAC | |
| | | ANGIHMNRGCPSAAVGYPGMKPQQHCPGEL | |
| | | QQQSDTSSLLRQTHLGNGYDPQSHQITRGP | |
| | | KSSPDEGSFLYTLPDDSTHQLLQPHHDCCQ | |
| | | RQEQPAAVGQSGVRRAPDSPVLEAVWDPPF | |
| | | HSGPPCCLGLVPVEEVDSPDSCQVSGGDWC | |
| | | PQHPVGAYVGQEPGMQLSPGPLVRVSFETP | |
| | | PLTI | |
| 60 | Q15018 | MAASISGYTFSAVCFHSANSNADHEGFLLG | ABRX2_HUMAN |
| | Entry version | EVRQEETFSISDSQISNTEFLQVIEIHNHQ | BRISC complex |
| | 142 | PCSKLFSFYDYASKVNEESLDRILKDRRKK | subunit Abraxas 2 |
| | (8 May 2019) | VIGWYRFRRNTQQQMSYREQVLHKQLTRIL | OS = Homo sapiens |
| | Sequence | GVPDLVFLLFSFISTANNSTHALEYVLFRP | OX = 9606 |
| | version 2 | NRRYNQRISLAIPNLGNTSQQEYKVSSVPN | GN = ABRAXAS2 |
| | (20 Dec. 2005) | TSQSYAKVIKEHGTDFFDKDGVMKDIRAIY | PE = 1 SV = 2 |
| | | QVYNALQEKVQAVCADVEKSERVVESCQAE | |
| | | VNKLRRQITQRKNEKEQERRLQQAVLSRQM | |
| | | PSESLDPAFSPRMPSSGFAAEGRSTLGDAE | |
| | | ASDPPPPYSDFHPNNQESTLSHSRMERSVF | |
| | | MPRPQAVGSSNYASTSAGLKYPGSGADLPP | |
| | | PQRAAGDSGEDSDDSDYENLIDPTEPSNSE | |
| | | YSHSKDSRPMAHPDEDPRNTQTSQI | |
| 61 | Q9BS26 | MHPAVFLSLPDLRCSLLLLVTWVFTPVTTE | ERP44_HUMAN |
| | Entry version | ITSLDTENIDEILNNADVALVNFYADWCRF | Endoplasmic |
| | 172 | SQMLHPIFEEASDVIKEEFPNENQVVFARV | reticulum resident |
| | (8 May 2019) | DCDQHSDIAQRYRISKYPTLKLFRNGMMMK | protein 44 OS = Homo |
| | Sequence | REYRGQRSVKALADYIRQQKSDPIQEIRDL | sapiens OX = 9606 |
| | version 1 | AEITTLDRSKRNIIGYFEQKDSDNYRVFER | GN = ERP44 PE = 1 |
| | (1 Jun. 2001) | VANILHDDCAFLSAFGDVSKPERYSGDNII | SV = 1 |
| | | YKPPGHSAPDMVYLGAMTNFDVTYNWIQDK | |
| | | CVPLVREITFENGEELTEEGLPFLILFHMK | |
| | | EDTESLEIFQNEVARQLISEKGTINFLHAD | |
| | | CDKFRHPLLHIQKTPADCPVIAIDSFRHMY | |
| | | VFGDFKDVLIPGKLKQFVFDLHSGKLHREF | |
| | | HHGPDPTDTAPGEQAQDVASSPPESSFQKL | |
| | | APSEYRYTLLRDRDEL | |
| 62 | O86476 | MKKRIDYLSNKQNKYSIRRFTVGTTSVIVG | CLFB_STAAE |
| | Entry version | ATILFGIGNHQAQASEQSNDTTQSSKNNAS | Clumping factor B |
| | 109 | ADSEKNNMIETPQLNTTANDTSDISANTNS | OS = Staphylococcus |
| | (8 May 2019) | ANVDSTTKPMSTQTSNTTTTEPASTNETPQ | aureus (strain |
| | Sequence | PTAIKNQATAAKMQDQTVPQEANSQVDNKT | Newman) |
| | version 2 | TNDANSIATNSELKNSQTLDLPQSSPQTIS | OX = 426430 GN = clfB |
| | (5 Feb. 2008) | NAQGTSKPSVRTRAVRSLAVAEPVVNAADA | PE = 1 SV = 2 |
| | | KGTNVNDKVTASNFKLEKTTFDPNQSGNTF | |
| | | MAANFTVTDKVKSGDYFTAKLPDSLTGNGD | |
| | | VDYSNSNNTMPIADIKSTNGDVVAKATYDI | |
| | | LTKTYTFVFTDYVNNKENINGQFSLPLFTD | |
| | | RAKAPKSGTYDANINIADEMFNNKITYNYS | |
| | | SPIAGIDKPNGANISSQIIGVDTASGQNTY | |
| | | KQTVFVNPKQRVLGNTWVYIKGYQDKIEES | |
| | | SGKVSATDTKLRIFEVNDTSKLSDSYYADP | |
| | | NDSNLKEVTDQFKNRIYYEHPNVASIKFGD | |
| | | ITKTYVVLVEGHYDNTGKNLKTQVIQENVD | |
| | | PVTNRDYSIFGWNNENVVRYGGGSADGDSA | |
| | | VNPKDPTPGPPVDPEPSPDPEPEPTPDPEP | |
| | | SPDPEPEPSPDPDPDSDSDSDSGSDSDSGS | |
| | | DSDSESDSDSDSDSDSDSDSDSESDSDSES | |
| | | DSDSDSDSDSDSDSDSDSDSDSDSDSDSDS | |
| | | DSDSESDSDSESDSESDSDSDSDSDSDSDS | |
| | | DSDSDSDSDSDSDSDSDSDSDSDSDSDSDS | |
| | | DSDSDSDSDSDSDSDSDSDSDSDSDSDSDS | |
| | | DSDSDSDSDSDSDSDSDSDSDSDSDSDSDS | |
| | | DSDSDSDSDSDSDSDSDSDSDSDSDSDSDS | |
| | | DSDSDSDSDSDSDSDSDSDSDSDSDSDSDS | |
| | | RVTPPNNEQKAPSNPKGEVNHSNKVSKQHK | |
| | | TDALPETGDKSENTNATLFGAMMALLGSLL | |
| | | LFRKRKQDHKEKA | |
| 63 | P45452 | MHPGVLAAFLFLSWTHCRALPLPSGGDEDD | MMP13_HUMAN |
| | Entry version | LSEEDLQFAERYLRSYYHPTNLAGILKENA | Collagenase 3 |
| | 196 | ASSMTERLREMQSFFGLEVTGKLDDNTLDV | OS = Homo sapiens |
| | (5 Jun. 2019) | MKKPRCGVPDVGEYNVFPRTLKWSKMNLTY | OX = 9606 |
| | Sequence | RIVNYTPDMTHSEVEKAFKKAFKVWSDVTP | GN = MMP13 PE = 1 |
| | version 1 | LNFTRLHDGIADIMISFGIKEHGDFYPFDG | SV = 1 |
| | (1 Nov. 1995) | PSGLLAHAFPPGPNYGGDAHFDDDETWTSS | |
| | | SKGYNLFLVAAHEFGHSLGLDHSKDPGALM | |
| | | FPIYTYTGKSHFMLPDDDVQGIQSLYGPGD | |
| | | EDPNPKHPKTPDKCDPSLSLDAITSLRGET | |
| | | MIFKDRFFWRLHPQQVDAELFLTKSFWPEL | |
| | | PNRIDAAYEHPSHDLIFIFRGRKFWALNGY | |
| | | DILEGYPKKISELGLPKEVKKISAAVHFED | |
| | | TGKTLLFSGNQVWRYDDTNHIMDKDYPRLI | |
| | | EEDFPGIGDKVDAVYEKNGYIYFFNGPIQF | |
| | | EYSIWSNRIVRVMPANSILWC | |
| 64 | Q9HC23 | MRSLCCAPLLLLLLLPPLLLTPRAGDAAVI | PROK2_HUMAN |
| | Entry version | TGACDKDSQCGGGMCCAVSIWVKSIRICTP | Prokineticin-2 |
| | 146 | MGKLGDSCHPLTRKNNFGNGRQERRKRKRS | OS = Homo sapiens |
| | (8 May 2019) | KRKKEVPFFGRRMHHTCPCLPGLACLRTSF | OX = 9606 |
| | Sequence | NRFICLAQK | GN = PROK2 PE = 1 |
| | version 2 | | SV = 2 |
| | (26 Sep. 2001) | | |
| 65 | Q13356 | MGKRQHQKDKMYITCAEYTHFYGGKKPDLP | PPIL2_HUMAN |
| | Entry version | QTNFRRLPFDHCSLSLQPFVYPVCTPDGIV | RING-type E3 |
| | 177 | FDLLNIVPWLKKYGTNPSNGEKLDGRSLIK | ubiquitin-protein |
| | (8 May 2019) | LNFSKNSEGKYHCPVLFTVFTNNTHIVAVR | ligase PPIL2 |
| | Sequence | TTGNVYAYEAVEQLNIKAKNFRDLLTDEPF | OS = Homo sapiens |
| | version 1 | SRQDIITLQDPTNLDKFNVSNFYHVKNNMK | OX = 9606 GN = PPIL2 |
| | (1 Nov. 1996) | IIDPDEEKAKQDPSYYLKNTNAETRETLQE | PE = 1 SV = 1 |
| | | LYKEFKGDEILAATMKAPEKKKVDKLNAAH | |
| | | YSTGKVSASFTSTAMVPETTHEAAAIDEDV | |
| | | LRYQFVKKKGYVRLHTNKGDLNLELHCDLT | |
| | | PKTCENFIRLCKKHYYDGTIFHRSIRNFVI | |
| | | QGGDPTGTGTGGESYWGKPFKDEFRPNLSH | |
| | | TGRGILSMANSGPNSNRSQFFITFRSCAYL | |
| | | DKKHTIFGRVVGGFDVLTAMENVESDPKTD | |
| | | RPKEEIRIDATTVFVDPYEEADAQIAQERK | |
| | | TQLKVAPETKVKSSQPQAGSQGPQTFRQGV | |
| | | GKYINPAATKRAAEEEPSTSATVPMSKKKP | |
| | | SRGFGDFSSW | |
| 66 | Q969J5 | MMPKHCFLGFLISFFLTGVAGTQSTHESLK | I22R2_HUMAN |
| | Entry version | PQRVQFQSRNFHNILQWQPGRALTGNSSVY | Interleukin-22 |
| | 144 | FVQYKIMFSCSMKSSHQKPSGCWQHISCNF | receptor subunit |
| | (8 May 2019) | PGCRTLAKYGQRQWKNKEDCWGTQELSCDL | alpha-2 OS = Homo |
| | Sequence | TSETSDIQEPYYGRVRAASAGSYSEWSMTP | sapiens OX = 9606 |
| | version 1 | RFTPWWETKIDPPVMNITQVNGSLLVILHA | GN = IL22RA2 PE = 1 |
| | (1 Dec. 2001) | PNLPYRYQKEKNVSIEDYYELLYRVFIINN | SV = 1 |
| | | SLEKEQKVYEGAHRAVEIEALTPHSSYCVV | |
| | | AEIYQPMLDRRSQRSEERCVEIP | |
| 67 | Q16585 | MAAAAAAAAEQQSSNGPVKKSMREKAVERR | SGCB_HUMAN Beta- |
| | Entry version | SVNKEHNSNFKAGYIPIDEDRLHKTGLRGR | sarcoglycan |
| | 166 | KGNLAICVIILLFILAVINLIITLVIWAVI | OS = Homo sapiens |
| | (8 May 2019) | RIGPNGCDSMEFHESGLLRFKQVSDMGVIH | OX = 9606 GN = SGCB |
| | Sequence | PLYKSTVGGRRNENLVITGNNQPIVFQQGT | PE = 1 SV = 1 |
| | version 1 | TKLSVENNKTSITSDIGMQFFDPRTQNILF | |
| | (1 Nov. 1996) | STDYETHEFHLPSGVKSLNVQKASTERITS | |
| | | NATSDLNIKVDGRAIVRGNEGVFIMGKTIE | |
| | | FHMGGNMELKAENSIILNGSVMVSTTRLPS | |
| | | SSSGDQLGSGDWVRYKLCMCADGTLFKVQV | |
| | | TSQNMGCQISDNPCGNTH | |
| 68 | Q14956 | MECLYYFLGFLLLAARLPLDAAKRFHDVLG | GPNMB_HUMAN |
| | Entry version | NERPSAYMREHNQLNGWSSDENDWNEKLYP | Transmembrane |
| | 157 | VWKRGDMRWKNSWKGGRVQAVLTSDSPALV | glycoprotein NMB |
| | (8 May 2019) | GSNITFAVNLIFPRCQKEDANGNIVYEKNC | OS = Homo sapiens |
| | Sequence | RNEAGLSADPYVYNWTAWSEDSDGENGTGQ | OX = 9606 |
| | version 2 | SHHNVFPDGKPFPHHPGWRRWNFIYVFHTL | GN = GPNMB PE = 1 |
| | (15 Mar. 2005) | GQYFQKLGRCSVRVSVNTANVTLGPQLMEV | SV = 2 |
| | | TVYRRHGRAYVPIAQVKDVYVVTDQIPVFV | |
| | | TMFQKNDRNSSDETFLKDLPIMFDVLIHDP | |
| | | SHFLNYSTINYKWSFGDNTGLFVSTNHTVN | |
| | | HTYVLNGTFSLNLTVKAAAPGPCPPPPPPP | |
| | | RPSKPTPSLATTLKSYDSNTPGPAGDNPLE | |
| | | LSRIPDENCQINRYGHFQATITIVEGILEV | |
| | | NIIQMTDVLMPVPWPESSLIDFVVTCQGSI | |
| | | PTEVCTIISDPTCEITQNTVCSPVDVDEMC | |
| | | LLTVRRTFNGSGTYCVNLTLGDDTSLALTS | |
| | | TLISVPDRDPASPLRMANSALISVGCLAIF | |
| | | VTVISLLVYKKHKEYNPIENSPGNVVRSKG | |
| | | LSVFLNRAKAVFFPGNQEKDPLLKNQEFKG | |
| | | VS | |
| 69 | O00300 | MNNLLCCALVFLDISIKWTTQETFPPKYLH | TR11B_HUMAN |
| | Entry version | YDEETSHQLLCDKCPPGTYLKQHCTAKWKT | Tumor necrosis factor |
| | 164 | VCAPCPDHYYTDSWHTSDECLYCSPVCKEL | receptor superfamily |
| | (8 May 2019) | QYVKQECNRTHNRVCECKEGRYLEIEFCLK | member 11B |
| | Sequence | HRSCPPGFGVVQAGTPERNTVCKRCPDGFF | OS = Homo sapiens |
| | version 3 | SNETSSKAPCRKHTNCSVFGLLLTQKGNAT | OX = 9606 |
| | (8 Feb. 2011) | HDNICSGNSESTQKCGIDVTLCEEAFFRFA | GN = TNFRSF11B |
| | | VPTKFTPNWLSVLVDNLPGTKVNAESVERI | PE = 1 SV = 3 |
| | | KRQHSSQEQTFQLLKLWKHQNKDQDIVKKI | |
| | | IQDIDLCENSVQRHIGHANLTFEQLRSLME | |
| | | SLPGKKVGAEDIEKTIKACKPSDQILKLLS | |
| | | LWRIKNGDQDTLKGLMHALKHSKTYHFPKT | |
| | | VTQSLKKTIRFLHSFTMYKLYQKLFLEMIG | |
| | | NQVQSVKISCL | |
| 70 | P55083 | MKALLALPLLLLLSTPPCAPQVSGIRGDAL | MFAP4_HUMAN |
| | Entry version | ERFCLQQPLDCDDIYAQGYQSDGVYLIYPS | Microfibril-associated |
| | 155 | GPSVPVPVFCDMTTEGGKWTVFQKRFNGSV | glycoprotein 4 |
| | (5 Jun. 2019) | SFFRGWNDYKLGFGRADGEYWLGLQNMHLL | OS = Homo sapiens |
| | Sequence | TLKQKYELRVDLEDFENNTAYAKYADFSIS | OX = 9606 |
| | version 2 | PNAVSAEEDGYTLFVAGFEDGGAGDSLSYH | GN = MFAP4 PE = 1 |
| | (1 Nov. 1997) | SGQKFSTFDRDQDLFVQNCAALSSGAFWFR | SV = 2 |
| | | SCHFANLNGFYLGGSHLSYANGINWAQWKG | |
| | | FYYSLKRTEMKIRRA | |
| 71 | P12110 | MLQGTCSVLLLWGILGAIQAQQQEVISPDT | CO6A2_HUMAN |
| | Entry version | TERNNNCPEKTDCPIHVYFVLDTSESVTMQ | Collagen alpha-2(VI) |
| | 215 | SPTDILLFHMKQFVPQFISQLQNEFYLDQV | chain OS = Homo |
| | (5 Jun. 2019) | ALSWRYGGLHFSDQVEVFSPPGSDRASFIK | sapiens OX = 9606 |
| | Sequence | NLQGISSFRRGTFTDCALANMTEQIRQDRS | GN = COL6A2 PE = 1 |
| | version 4 | KGTVHFAVVITDGHVTGSPCGGIKLQAERA | SV = 4 |
| | (6 Feb. 2007) | REEGIRLFAVAPNQNLKEQGLRDIASTPHE | |
| | | LYRNDYATMLPDSTEIDQDTINRIIKVMKH | |
| | | EAYGECYKVSCLEIPGPSGPKGYRGQKGAK | |
| | | GNMGEPGEPGQKGRQGDPGIEGPIGFPGPK | |
| | | GVPGFKGEKGEFGADGRKGAPGLAGKNGTD | |
| | | GQKGKLGRIGPPGCKGDPGNRGPDGYPGEA | |
| | | GSPGERGDQGGKGDPGRPGRRGPPGEIGAK | |
| | | GSKGYQGNSGAPGSPGVKGAKGGPGPRGPK | |
| | | GEPGRRGDPGTKGSPGSDGPKGEKGDPGPE | |
| | | GPRGLAGEVGNKGAKGDRGLPGPRGPQGAL | |
| | | GEPGKQGSRGDPGDAGPRGDSGQPGPKGDP | |
| | | GRPGFSYPGPRGAPGEKGEPGPRGPEGGRG | |
| | | DFGLKGEPGRKGEKGEPADPGPPGEPGPRG | |
| | | PRGVPGPEGEPGPPGDPGLTECDVMTYVRE | |
| | | TCGCCDCEKRCGALDVVFVIDSSESIGYTN | |
| | | FTLEKNFVINVVNRLGAIAKDPKSETGTRV | |
| | | GVVQYSHEGTFEAIQLDDERIDSLSSFKEA | |
| | | VKNLEWIAGGTWTPSALKFAYDRLIKESRR | |
| | | QKTRVFAVVITDGRHDPRDDDLNLRALCDR | |
| | | DVTVTAIGIGDMFHEKHESENLYSIACDKP | |
| | | QQVRNMTLFSDLVAEKFIDDMEDVLCPDPQ | |
| | | IVCPDLPCQTELSVAQCTQRPVDIVFLLDG | |
| | | SERLGEQNFHKARRFVEQVARRLTLARRDD | |
| | | DPLNARVALLQFGGPGEQQVAFPLSHNLTA | |
| | | IHEALETTQYLNSFSHVGAGVVHAINAIVR | |
| | | SPRGGARRHAELSFVFLTDGVTGNDSLHES | |
| | | AHSMRKQNVVPTVLALGSDVDMDVLTTLSL | |
| | | GDRAAVFHEKDYDSLAQPGFFDRFIRWIC | |
| 72 | O00472 | MAAGGTGGLREEQRYGLSCGRLGQDNITVL | ELL2_HUMAN RNA |
| | Entry version | HVKLTETAIRALETYQSHKNLIPFRPSIQF | polymerase II |
| | 150 | QGLHGLVKIPKNDPLNEVHNFNFYLSNVGK | elongation factor |
| | (8 May 2019) | DNPQGSFDCIQQTFSSSGASQLNCLGFIQD | ELL2 OS = Homo |
| | Sequence | KITVCATNDSYQMTRERMTQAEEESRNRST | sapiens OX = 9606 |
| | version 2 | KVIKPGGPYVGKRVQIRKAPQAVSDTVPER | GN = ELL2 PE = 1 |
| | (28 Jul. 2009) | KRSTPMNPANTIRKTHSSSTISQRPYRDRV | SV = 2 |
| | | IHLLALKAYKKPELLARLQKDGVNQKDKNS | |
| | | LGAILQQVANLNSKDLSYTLKDYVFKELQR | |
| | | DWPGYSEIDRRSLESVLSRKLNPSQNAAGT | |
| | | SRSESPVCSSRDAVSSPQKRLLDSEFIDPL | |
| | | MNKKARISHLTNRVPPTLNGHLNPTSEKSA | |
| | | AGLPLPPAAAAIPTPPPLPSTYLPISHPPQ | |
| | | IVNSNSNSPSTPEGRGTQDLPVDSFSQNDS | |
| | | IYEDQQDKYTSRTSLETLPPGSVLLKCPKP | |
| | | MEENHSMSHKKSKKKSKKHKEKDQIKKHDI | |
| | | ETIEEKEEDLKREEEIAKLNNSSPNSSGGV | |
| | | KEDCTASMEPSAIELPDYLIKYIAIVSYEQ | |
| | | RQNYKDDFNAEYDEYRALHARMETVARRFI | |
| | | KLDAQRKRLSPGSKEYQNVHEEVLQEYQKI | |
| | | KQSSPNYHEEKYRCEYLHNKLAHIKRLIGE | |
| | | FDQQQAESWS | |
| 73 | O75071 | MKKRKELNALIGLAGDSRRKKPKKGPSSHR | EFC14_HUMAN EF- |
| | Entry version | LLRTEPPDSDSESSSEEEEEFGVVGNRSRF | hand calcium-binding |
| | 149 | AKGDYLRCCKICYPLCGFVILAACVVACVG | domain-containing |
| | (8 May 2019) | LVWMQVALKEDLDALKEKFRTMESNQKSSF | protein 14 OS = Homo |
| | Sequence | QEIPKLNEELLSKQKQLEKIESGEMGLNKV | sapiens OX = 9606 |
| | version 1 | WINITEMNKQISLLTSAVNHLKANVKSAAD | GN = EFCAB14 PE = 1 |
| | (1 Nov. 1998) | LISLPTTVEGLQKSVASIGNTLNSVHLAVE | SV = 1 |
| | | ALQKTVDEHKKTMELLQSDMNQHFLKETPG | |
| | | SNQIIPSPSATSELDNKTHSENLKQDILYL | |
| | | HNSLEEVNSALVGYQRQNDLKLEGMNETVS | |
| | | NLTQRVNLIESDVVAMSKVEKKANLSFSMM | |
| | | GDRSATLKRQSLDQVTNRTDTVKIQSIKKE | |
| | | DSSNSQVSKLREKLQLISALTNKPESNRPP | |
| | | ETADEEQVESFTSKPSALPKFSQFLGDPVE | |
| | | KAAQLRPISLPGVSSTEDLQDLFRKTGQDV | |
| | | DGKLTYQEIWTSLGSAMPEPESLRAFDSDG | |
| | | DGRYSFLELRVALGI | |
| 74 | Q9H4I9 | MASGAARWLVLAPVRSGALRSGPSLRKDGD | EMRE_HUMAN |
| | Entry version | VSAAWSGSGRSLVPSRSVIVTRSGAILPKP | Essential MCU |
| | 116 | VKMSFGLLRVFSIVIPFLYVGTLISKNFAA | regulator, |
| | (8 May 2019) | LLEEHDIFVPEDDDDDD | mitochondrial |
| | Sequence | | OS = Homo sapiens |
| | version 1 | | OX = 9606 |
| | (1 Mar. 2001) | | GN = SMDT1 PE = 1 |
| | | | SV = 1 |
| 75 | P52292 | MSTNENANTPAARLHRFKNKGKDSTEMRRR | IMA1_HUMAN |
| | Entry version | RIEVNVELRKAKKDDQMLKRRNVSSFPDDA | Importin subunit |
| | 223 | TSPLQENRNNQGTVNWSVDDIVKGINSSNV | alpha-1 OS = Homo |
| | (5 Jun. 2019) | ENQLQATQAARKLLSREKQPPIDNIIRAGL | sapiens OX = 9606 |
| | Sequence | IPKFVSFLGRTDCSPIQFESAWALTNIASG | GN = KPNA2 PE = 1 |
| | version 1 | TSEQTKAVVDGGAIPAFISLLASPHAHISE | SV = 1 |
| | (1 Oct. 1996) | QAVWALGNIAGDGSVFRDLVIKYGAVDPLL | |
| | | ALLAVPDMSSLACGYLRNLTWTLSNLCRNK | |
| | | NPAPPIDAVEQILPTLVRLLHHDDPEVLAD | |
| | | TCWAISYLTDGPNERIGMVVKTGVVPQLVK | |
| | | LLGASELPIVTPALRAIGNIVTGTDEQTQV | |
| | | VIDAGALAVFPSLLTNPKTNIQKEATWTMS | |
| | | NITAGRQDQIQQVVNHGLVPFLVSVLSKAD | |
| | | FKTQKEAVWAVTNYTSGGTVEQIVYLVHCG | |
| | | IIEPLMNLLTAKDTKIILVILDAISNIFQA | |
| | | AEKLGETEKLSIMIEECGGLDKIEALQNHE | |
| | | NESVYKASLSLIEKYFSVEEEEDQNVVPET | |
| | | TSEGYTFQVQDGAPGTFNF | |
| 76 | Q9BY71 | MGTVRPPRPSLLLVSTRESCLFLLFCLHLG | LRRC3_HUMAN |
| | Entry version | AACPQPCRCPDHAGAVAVFCSLRGLQEVPE | Leucine-rich repeat- |
| | 139 | DIPANTVLLKLDANKISHLPDGAFQHLHRL | containing protein 3 |
| | (8 May 2019) | RELDLSHNAIEAIGSATFAGLAGGLRLLDL | OS = Homo sapiens |
| | Sequence | SYNRIQRIPKDALGKLSAKIRLSHNPLHCE | OX = 9606 |
| | version 1 | CALQEALWELKLDPDSVDEIACHTSVQEEF | GN = LRRC3 PE = 1 |
| | (1 Jun. 2001) | VGKPLVQALDAGASLCSVPHRTTDVAMLVT | SV = 1 |
| | | MFGWFAMVIAYVVYYVRHNQEDARRHLEYL | |
| | | KSLPSAPASKDPIGPGP | |
| 77 | Q96IQ7 | MAELPGPFLCGALLGFLCLSGLAVEVKVPT | VSIG2_HUMAN V-set |
| | Entry version | EPLSTPLGKTAELTCTYSTSVGDSFALEWS | and immunoglobulin |
| | 138 | FVQPGKPISESHPILYFTNGHLYPTGSKSK | domain-containing |
| | (8 May 2019) | RVSLLQNPPTVGVATLKLTDVHPSDTGTYL | protein 2 OS = Homo |
| | Sequence | CQVNNPPDFYTNGLGLINLTVLVPPSNPLC | sapiens OX = 9606 |
| | version 1 | SQSGQTSVGGSTALRCSSSEGAPKPVYNWV | GN = VSIG2 PE = 1 |
| | (1 Dec. 2001) | RLGTFPTPSPGSMVQDEVSGQLILTNLSLT | SV = 1 |
| | | SSGTYRCVATNQMGSASCELTLSVTEPSQG | |
| | | RVAGALIGVLLGVLLLSVAAFCLVRFQKER | |
| | | GKKPKETYGGSDLREDAIAPGISEHTCMRA | |
| | | DSSKGFLERPSSASTVTTTKSKLPMVV | |
| 78 | Q1W4C9 | MAAFPHKIIFFLVCSTLTHVAFSGIFNKRD | ISK13_HUMAN |
| | Entry version | FTRWPKPRCKMYIPLDPDYNADCPNVTAPV | Serine protease |
| | 96 | CASNGHTFQNECFFCVEQREFHYRIKFEKY | inhibitor Kazal-type |
| | (8 May 2019) | GKCD | 13 OS = Homo sapiens |
| | Sequence | | OX = 9606 |
| | version 1 | | GN = SPINK13 PE = 3 |
| | (2 May 2006) | | SV = 1 |
| 79 | P08833 | MSEVPVARVWLVLLLLTVQVGVTAGAPWQC | IBP1_HUMAN |
| | Entry version | APCSAEKLALCPPVSASCSEVTRSAGCGCC | Insulin-like growth |
| | 207 | PMCALPLGAACGVATARCARGLSCRALPGE | factor-binding protein |
| | (8 May 2019) | QQPLHALTRGQGACVQESDASAPHAAEAGS | 1 OS = Homo sapiens |
| | Sequence | PESPESTEITEEELLDNFHLMAPSEEDHSI | OX = 9606 |
| | version 1 | LWDAISTYDGSKALHVTNIKKWKEPCRIEL | GN = IGFBP1 PE = 1 |
| | (1 Nov. 1988) | YRVVESLAKAQETSGEEISKFYLPNCNKNG | SV = 1 |
| | | FYHSRQCETSMDGEAGLCWCVYPWNGKRIP | |
| | | GSPEIRGDPNCQIYFNVQN | |
| 80 | Q8N688 | MKLLLLTLTVLLLLSQLTPGGTQRCWNLYG | DB123_HUMAN |
| | Entry version | KCRYRCSKKERVYVYCINNKMCCVKPKYQP | Beta-defensin 123 |
| | 119 | KERWWPF | OS = Homo sapiens |
| | (8 May 2019) | | OX = 9606 |
| | Sequence | | GN = DEFB123 PE = 2 |
| | version 1 | | SV = 1 |
| | (1 Oct. 2002) | | |
| 81 | Q9BWV2 | MPIKPVGWICGQVLKNFSGRIEGIQKAIMD | SPAT9_HUMAN |
| | Entry version | LVDEFKDEFPTILRLSQSNQKREPAQKTSK | Spermatogenesis- |
| | 103 | IRMAIALAKINRATLIRGLNSISRSSKSVA | associated protein 9 |
| | (8 May 2019) | KLLHPQLACRLLELRDISGRLLREVNAPRQ | OS = Homo sapiens |
| | Sequence | PLYNIQVRKGSLFEIISFPAKTALTSIIYA | OX = 9606 |
| | version 2 | SYAALIYLAVCVNAVLKKVKNIFQEEESIR | GN = SPATA9 PE = 2 |
| | (20 Feb. 2007) | QNREESENCRKAFSEPVLSEPMFAEGEIKA | SV = 2 |
| | | KPYRSLPEKPDISDYPKLLANKQSNNIQVL | |
| | | HSVFDQSAEMNEQI | |
| 82 | P61978 | METEQPEETFPNTETNGEFGKRPAEDMEEE | HNRPK_HUMAN |
| | Entry version | QAFKRSRNTDEMVELRILLQSKNAGAVIGK | Heterogeneous |
| | 190 | GGKNIKALRTDYNASVSVPDSSGPERILSI | nuclear |
| | (5 Jun. 2019) | SADIETIGEILKKIIPTLEEGLQLPSPTAT | ribonucleoprotein K |
| | Sequence | SQLPLESDAVECLNYQHYKGSDFDCELRLL | OS = Homo sapiens |
| | version 1 | IHQSLAGGIIGVKGAKIKELRENTQTTIKL | OX = 9606 |
| | (7 Jun. 2004) | FQECCPHSTDRVVLIGGKPDRVVECIKIIL | GN = HNRNPK PE = 1 |
| | | DLISESPIKGRAQPYDPNFYDETYDYGGFT | SV = 1 |
| | | MMFDDRRGRPVGFPMRGRGGFDRMPPGRGG | |
| | | RPMPPSRRDYDDMSPRRGPPPPPPGRGGRG | |
| | | GSRARNLPLPPPPPPRGGDLMAYDRRGRPG | |
| | | DRYDGMVGFSADETWDSAIDTWSPSEWQMA | |
| | | YEPQGGSGYDYSYAGGRGSYGDLGGPIITT | |
| | | QVTIPKDLAGSIIGKGGQRIKQIRHESGAS | |
| | | IKIDEPLEGSEDRIITITGTQDQIQNAQYL | |
| | | LQNSVKQYSGKFF | |
| 83 | Q9UJU6 | MAANLSRNGPALQEAYVRVVTEKSPTDWAL | DBNL_HUMAN |
| | Entry version | FTYEGNSNDIRVAGTGEGGLEEMVEELNSG | Drebrin-like protein |
| | 175 | KVMYAFCRVKDPNSGLPKFVLINWTGEGVN | OS = Homo sapiens |
| | (8 May 2019) | DVRKGACASHVSTMASFLKGAHVTINARAE | OX = 9606 GN = DBNL |
| | Sequence | EDVEPECIMEKVAKASGANYSFHKESGRFQ | PE = 1 SV = 1 |
| | version 1 | DVGPQAPVGSVYQKTNAVSEIKRVGKDSFW | |
| | (1 May 2000) | AKAEKEEENRRLEEKRRAEEAQRQLEQERR | |
| | | ERELREAARREQRYQEQGGEASPQRTWEQQ | |
| | | QEVVSRNRNEQESAVHPREIFKQKERAMST | |
| | | TSISSPQPGKLRSPFLQKQLTQPETHFGRE | |
| | | PAAAISRPRADLPAEEPAPSTPPCLVQAEE | |
| | | EAVYEEPPEQETFYEQPPLVQQQGAGSEHI | |
| | | DHHIQGQGLSGQGLCARALYDYQAADDTEI | |
| | | SFDPENLITGIEVIDEGWWRGYGPDGHFGM | |
| | | FPANYVELIE | |
| 84 | O43323 | MALLTNLLPLCCLALLALPAQSCGPGRGPV | DHH_HUMAN Desert |
| | Entry version | GRRRYARKQLVPLLYKQFVPGVPERTLGAS | hedgehog protein |
| | 178 | GPAEGRVARGSERFRDLVPNYNPDIIFKDE | OS = Homo sapiens |
| | (5 Jun. 2019) | ENSGADRLMTERCKERVNALAIAVMNMWPG | OX = 9606 GN = DHH |
| | Sequence | VRLRVTEGWDEDGHHAQDSLHYEGRALDIT | PE = 1 SV = 1 |
| | version 1 | TSDRDRNKYGLLARLAVEAGFDWVYYESRN | |
| | (1 Jun. 1998) | HVHVSVKADNSLAVRAGGCFPGNATVRLWS | |
| | | GERKGLRELHRGDWVLAADASGRVVPTPVL | |
| | | LFLDRDLQRRASFVAVETEWPPRKLLLTPW | |
| | | HLVFAARGPAPAPGDFAPVFARRLRAGDSV | |
| | | LAPGGDALRPARVARVAREEAVGVFAPLTA | |
| | | HGTLLVNDVLASCYAVLESHQWAHRAFAPL | |
| | | RLLHALGALLPGGAVQPTGMHWYSRLLYRL | |
| | | AEELLG | |
| 85 | P08476 | MPLLWLRGFLLASCWIIVRSSPTPGSEGHS | INHBA_HUMAN |
| | Entry version | AAPDCPSCALAALPKDVPNSQPEMVEAVKK | Inhibin beta A chain |
| | 209 | HILNMLHLKKRPDVTQPVPKAALLNAIRKL | OS = Homo sapiens |
| | (8 May 2019) | HVGKVGENGYVEIEDDIGRRAEMNELMEQT | OX = 9606 GN = INHBA |
| | Sequence | SEIITFAESGTARKTLHFEISKEGSDLSVV | PE = 1 SV = 2 |
| | version 2 | ERAEVWLFLKVPKANRTRTKVTIRLFQQQK | |
| | (1 Jul. 1989) | HPQGSLDTGEEAEEVGLKGERSELLLSEKV | |
| | | VDARKSTWHVFPVSSSIQRLLDQGKSSLDV | |
| | | RIACEQCQESGASLVLLGKKKKKEEEGEGK | |
| | | KKGGGEGGAGADEEKEQSHRPFLMLQARQS | |
| | | EDHPHRRRRRGLECDGKVNICCKKQFFVSF | |
| | | KDIGWNDWIIAPSGYHANYCEGECPSHIAG | |
| | | TSGSSLSFHSTVINHYRMRGHSPFANLKSC | |
| | | CVPTKLRPMSMLYYDDGQNIIKKDIQNMIV | |
| | | EECGCS | |
| 86 | P09529 | MDGLPGRALGAACLLLLAAGWLGPEAWGSP | INHBB_HUMAN |
| | Entry version | TPPPTPAAPPPPPPPGSPGGSQDTCTSCGG | Inhibin beta B chain |
| | 176 | FRRPEELGRVDGDFLEAVKRHILSRLQMRG | OS = Homo sapiens |
| | (8 May 2019) | RPNITHAVPKAAMVTALRKLHAGKVREDGR | OX = 9606 GN = INHBB |
| | Sequence | VEIPHLDGHASPGADGQERVSEIISFAETD | PE = 1 SV = 2 |
| | version 2 | GLASSRVRLYFFISNEGNQNLFVVQASLWL | |
| | (1 Oct. 1996) | YLKLLPYVLEKGSRRKVRVKVYFQEQGHGD | |
| | | RWNMVEKRVDLKRSGWHTFPLTEAIQALFE | |
| | | RGERRLNLDVQCDSCQELAVVPVFVDPGEE | |
| | | SHRPFVVVQARLGDSRHRIRKRGLECDGRT | |
| | | NLCCRQQFFIDFRLIGWNDWIIAPTGYYGN | |
| | | YCEGSCPAYLAGVPGSASSFHTAVVNQYRM | |
| | | RGLNPGTVNSCCIPTKLSTMSMLYFDDEYN | |
| | | IVKRDVPNMIVEECGCA | |
| 87 | Q99969 | MRRLLIPLALWLGAVGVGVAELTEAQRRGL | RARR2_HUMAN |
| | Entry version | QVALEEFHKHPPVQWAFQETSVESAVDTPF | Retinoic acid receptor |
| | 149 | PAGIFVRLEFKLQQTSCRKRDWKKPECKVR | responder protein 2 |
| | (5 Jun. 2019) | PNGRKRKCLACIKLGSEDKVLGRLVHCPIE | OS = Homo sapiens |
| | Sequence | TQVLREAEEHQETQCLRVQRAGEDPHSFYF | OX = 9606 |
| | version 1 | PGQFAFSKALPRS | GN = RARRES2 PE = 1 |
| | (1 May 1997) | | SV = 1 |
| 88 | P22888 | MKQRFSALQLLKLLLLLQPPLPRALREALC | LSHR_HUMAN |
| | Entry version | PEPCNCVPDGALRCPGPTAGLTRLSLAYLP | Lutropin- |
| | 210 | VKVIPSQAFRGLNEVIKIEISQIDSLERIE | choriogonadotropic |
| | (8 May 2019) | ANAFDNLLNLSEILIQNTKNLRYIEPGAFI | hormone receptor |
| | Sequence | NLPRLKYLSICNTGIRKFPDVTKVFSSESN | OS = Homo sapiens |
| | version 4 | FILEICDNLHITTIPGNAFQGMNNESVTLK | OX = 9606 |
| | (15 Dec. 2009) | LYGNGFEEVQSHAFNGTTLTSLELKENVHL | GN = LHCGR PE = 1 |
| | | EKMHNGAFRGATGPKTLDISSTKLQALPSY | SV = 4 |
| | | GLESIQRLIATSSYSLKKLPSRETFVNLLE | |
| | | ATLTYPSHCCAFRNLPTKEQNFSHSISENF | |
| | | SKQCESTVRKVNNKTLYSSMLAESELSGWD | |
| | | YEYGFCLPKTPRCAPEPDAFNPCEDIMGYD | |
| | | FLRVLIWLINILAIMGNMTVLFVLLTSRYK | |
| | | LTVPRFLMCNLSFADFCMGLYLLLIASVDS | |
| | | QTKGQYYNHAIDWQTGSGCSTAGFFTVFAS | |
| | | ELSVYTLTVITLERWHTITYAIHLDQKLRL | |
| | | RHAILIMLGGWLFSSLIAMLPLVGVSNYMK | |
| | | VSICFPMDVETTLSQVYILTILILNVVAFF | |
| | | IICACYIKIYFAVRNPELMATNKDTKIAKK | |
| | | MAILIFTDFTCMAPISFFAISAAFKVPLIT | |
| | | VTNSKVLLVLFYPINSCANPFLYAIFTKTF | |
| | | QRDFFLLLSKFGCCKRRAELYRRKDFSAYT | |
| | | SNCKNGFTGSNKPSQSTLKLSTLHCQGTAL | |
| | | LDKTRYTEC | |
| 89 | P35443 | MLAPRGAAVLLLHLVLQRWLAAGAQATPQV | TSP4_HUMAN |
| | Entry version | FDLLPSSSQRLNPGALLPVLTDPALNDLYV | Thrombospondin-4 |
| | 186 | ISTFKLQTKSSATIFGLYSSTDNSKYFEFT | OS = Homo sapiens |
| | (8 May 2019) | VMGRLNKAILRYLKNDGKVHLVVFNNLQLA | OX = 9606 |
| | Sequence | DGRRHRILLRLSNLQRGAGSLELYLDCIQV | GN = THBS4 PE = 1 |
| | version 2 | DSVHNLPRAFAGPSQKPETIELRTFQRKPQ | SV = 2 |
| | (23 Nov. 2004) | DFLEELKLVVRGSLFQVASLQDCFLQQSEP | |
| | | LAATGTGDFNRQFLGQMTQLNQLLGEVKDL | |
| | | LRQQVKETSFLRNTIAECQACGPLKFQSPT | |
| | | PSTVVPPAPPAPPTRPPRRCDSNPCFRGVQ | |
| | | CTDSRDGFQCGPCPEGYTGNGITCIDVDEC | |
| | | KYHPCYPGVHCINLSPGFRCDACPVGFTGP | |
| | | MVQGVGISFAKSNKQVCTDIDECRNGACVP | |
| | | NSICVNTLGSYRCGPCKPGYTGDQIRGCKA | |
| | | ERNCRNPELNPCSVNAQCIEERQGDVTCVC | |
| | | GVGWAGDGYICGKDVDIDSYPDEELPCSAR | |
| | | NCKKDNCKYVPNSGQEDADRDGIGDACDED | |
| | | ADGDGILNEQDNCVLIHNVDQRNSDKDIFG | |
| | | DACDNCLSVLNNDQKDTDGDGRGDACDDDM | |
| | | DGDGIKNILDNCPKFPNRDQRDKDGDGVGD | |
| | | ACDSCPDVSNPNQSDVDNDLVGDSCDTNQD | |
| | | SDGDGHQDSTDNCPTVINSAQLDTDKDGIG | |
| | | DECDDDDDNDGIPDLVPPGPDNCRLVPNPA | |
| | | QEDSNSDGVGDICESDFDQDQVIDRIDVCP | |
| | | ENAEVTLTDFRAYQTVVLDPEGDAQIDPNW | |
| | | VVLNQGMEIVQTMNSDPGLAVGYTAFNGVD | |
| | | FEGTFHVNTQTDDDYAGFIFGYQDSSSFYV | |
| | | VMWKQTEQTYWQATPFRAVAEPGIQLKAVK | |
| | | SKTGPGEHLRNSLWHTGDTSDQVRLLWKDS | |
| | | RNVGWKDKVSYRWFLQHRPQVGYIRVRFYE | |
| | | GSELVADSGVTIDTTMRGGRLGVFCFSQEN | |
| | | IIWSNLKYRCNDTIPEDFQEFQTQNFDRFD | |
| | | N | |
| 90 | P14136 | MERRRITSAARRSYVSSGEMMVGGLAPGRR | GFAP_HUMAN Glial |
| | Entry version | LGPGTRLSLARMPPPLPTRVDFSLAGALNA | fibrillary acidic |
| | 207 | GFKETRASERAEMMELNDRFASYIEKVRFL | protein |
| | (5 Jun. 2019) | EQQNKALAAELNQLRAKEPTKLADVYQAEL | OS = Homo sapiens |
| | Sequence | RELRLRLDQLTANSARLEVERDNLAQDLAT | OX = 9606 GN = GFAP |
| | version 1 | VRQKLQDETNLRLEAENNLAAYRQEADEAT | PE = 1 SV = 1 |
| | (1 Jan. 1990) | LARLDLERKIESLEEEIRFLRKIHEEEVRE | |
| | | LQEQLARQQVHVELDVAKPDLTAALKEIRT | |
| | | QYEAMASSNMHEAEEWYRSKFADLTDAAAR | |
| | | NAELLRQAKHEANDYRRQLQSLTCDLESLR | |
| | | GTNESLERQMREQEERHVREAASYQEALAR | |
| | | LEEEGQSLKDEMARHLQEYQDLLNVKLALD | |
| | | IEIATYRKLLEGEENRITIPVQTFSNLQIR | |
| | | ETSLDTKSVSEGHLKRNIVVKTVEMRDGEV | |
| | | IKESKQEHKDVM | |
| 91 | Q7Z6M3 | MWSHLNRLLF WSIFSSVTCR | MILR1_HUMAN |
| | Entry version | KAVLDCEAMK TNEFPSPCLD | Allergin-1 OS = Homo |
| | 110 | SKTKVVMKGQ | sapiens OX = 9606 |
| | (5 Jun. 2019) | NVSMFCSHKN KSLQITYSLF | GN = MILR1 PE = 1 |
| | Sequence | RRKTHLGTQD GKGEPAIFNL | SV = 2 |
| | version 2 | SITEAHESGP | |
| | (23 Oct. 2007) | YKCKAQVTSC SKYSRDFSFT | |
| | | IVDPVTSPVL NIMVIQTETD | |
| | | RHITLHCLSV | |
| | | NGSLPINYTF FENHVAISPA | |
| | | ISKYDREPAE FNLTKKNPGE | |
| | | EEEYRCEAKN | |
| | | RLPNYATYSH PVTMPSTGGD | |
| | | SCPFCLKLLL PGLLLLLVVI | |
| | | ILILAFWVLP | |
| | | KYKTRKAMRN NVPRDRGDTA | |
| | | MEVGIYANIL EKQAKEESVP | |
| | | EVGSRPCVST | |
| | | AQDEAKHSQE LQYATPVFQE | |
| | | VAPREQEACD SYKSGYVYSE | |
| | | LNF | |

### EXAMPLES

### Materials & Methods

CSF samples were collected from 41 patients with CDMS, 71 patients with CIS, and 64 non-MS controls and analysed using a omics methodology, including multi-omics methodology. This approach used nuclear magnetic resonance spectroscopy to measure over 100 CSF and serum metabolite concentrations and an aptamer-based proteomics assay (SOMAscan) to measure over 5000 CSF protein levels combined with multivariate feature selection and pathway analysis.

### Biofluid Sample Collection

Plasma: Blood was collected into BD vacutainer lithium-heparin tubes (product number 367375) and stored at room temperature for 30 mins before centrifugation at 2,200×g for 10 mins and plasma immediately aliquoted and stored at −80° C. Serum: Blood was collected into BD additive free tubes (product number) and stored at room temperature for 30 mins before centrifugation at 2,200×g for 10 mins and serum immediately aliquoted and stored at −80° C. Cerebrospinal fluid (CSF): samples were collected in to additive free tubes and immediately aliquoted and stored at −80° C.

### Proteomics Analysis of CSF Samples

Proteomic profiles were characterised using the SOMAscan Assay (SomaLogic, Inc.; Boulder, Colo., USA) at the Trans-NIH Center for Human Immunology, Autoimmunity, and Inflammation (CHI), National Institutes of Health (Bethesda, Md., USA) as previously described (Proc. Natl. Acad. Sci. U.S.A. 109, 19971-19976 (2012), Proc. Natl. Acad. Sci. U.S.A. 112, 7153-7158 (2015), Science. 2018 Aug. 24; 361(6404):769-773.).

In brief, 5,457 individual serum samples were treated with the detergent Tween-20 to prevent loss of reagent material to tube walls and for lysis of exosomes, and then incubated with the mixture of 5,034 SOMAmers to generate SOMAmerprotein complexes. Unbound SOMAmers and unbound or non-specifically bound proteins were eliminated by 2 bead-based immobilization wash steps and the use of polyanionic competitors. After eluting the enriched SOMAmers from their target proteins they were directly quantified on an Agilent hybridization array (Agilent Technologies). Hybridization controls were used to correct for systematic variability in detection and calibrator samples of three dilution sets (40%, 1% and 0.005%) were included so that the degree of fluorescence was a quantitative reflection of protein concentration. All scale factors were then used to normalize the protein data. To avoid batch or time of processing biases, both sample collection and sample processing for protein measurements were randomized and all samples run as a single set. The 5,034 SOMAmers that passed quality control had median intra-assay and inter-assay coefficient of variation, CV=100×σ/μ, or similar to that reported on variability in the 4 SOMAscan assays (Sci. Rep. 7, 14248 (2017)).

### NMR Metabolomics Analysis

NMR metabolomics analysis of CSF/serum/plasma was carried out as previously described (J Crohns Colitis. 2018 Nov. 15; 12(11):1326-1337., Acta Neuropathol Commun. 2017 Dec. 6; 5(1):95.) and is discussed in more detail below.

### NMR Sample Preparation

Plasma/serum samples were defrosted at room temperature and centrifuged at 100,000×g for 30 minutes at 4° C. 150 μL of the plasma/serum supernatant was then diluted with 450 μL of 75 mM sodium phosphate buffer prepared in D₂O (pH 7.4). Samples were then centrifuged at 16,000×g for 3 minutes to remove any precipitate before transferring to a 5-mm NMR tube.

CSF samples were defrosted at room temperature and centrifuged at 100,000×g for 30 minutes at 4° C. 75 μL of the CSF supernatant was then diluted with 525 μL of 75 mM sodium phosphate buffer prepared in D₂O (pH 7.4)

### NMR Spectroscopy

All NMR spectra were acquired using a 700-MHz Bruker AVII spectrometer operating at 16.4 T equipped with a ¹H (¹³C/¹⁵N) TCI cryoprobe. Sample temperature was stable at 310K. ¹H NMR spectra were acquired using a 1D NOESY presaturation scheme for attenuation of the water resonance with a 2 s presaturation. A spin-echo Carr-Purcell-Meiboom-Gill (CPMG) sequence with a T interval of 400 μs, 80 loops, 32 data collections, an acquisition time of 1.5 s, a relaxation delay of 2 s, and a fixed receiver gain was used to supress broad signals arising from large molecular weight plasma components. ¹H correlation spectroscopy (COSY, TOCSY) spectra were acquired on at least one sample in each classification to aid in metabolite identification. For quality control, pooled plasma samples were spread throughout the run to monitor technical variation.

### NMR Data Preprocessing

Resulting free induction decays (FIDs) were zero-filled by a factor of 2 and multiplied by an exponential function corresponding to 0.30 Hz line broadening prior to Fourier transformation. All spectra were phased, baseline corrected (using a 3^{rd} degree polynomial), and chemical shifts referenced to the lactate-CHs doublet resonance at δ=1.33 ppm in Topspin 2.1 (Bruker, Germany). Spectra were visually examined for errors in baseline correction, referencing, spectral distortion, or contamination and then exported to ACD/Labs Spectrus Processor Academic Edition 12.01 (Advanced Chemistry Development, Inc.). The regions of the spectra between 0.08-4.20 ppm and 5.20-8.50 ppm were divided in to 0.02 ppm width ‘buckets’ and the absolute value of the integral of each spectral bucket was Pareto scaled. Resonances were assigned by reference to literature values [Anal Biochem 325:260-272, J Pharm Biomed Anal 33:1103-1115] and the Human Metabolome Database [Nucleic Acids Res 41:D801-807. doi:10.1093/nar/gks1065, Nucleic Acids Res 37:D603-610. doi:10.1093/nar/gkn810, Nucleic Acids Res 35:D521-526. doi:10.1093/nar/gk1923] and further confirmed by inspection of the 2D spectra, spiking of known compounds, and 1D-TOCSY spectra.

### Statistical Analysis

The bucketed integrals were imported into R (R foundation for statistical computing, Vienna, Austria) [Team RC R: A Language and Environment for Statistical Computing. R Found Stat Comput]. All multivariate analysis was carried out using in-house R scripts and the ropls package [J Proteome Res 14:3322-3335]. Principal component analysis (PCA) was used to visualize the degree of separation between the disease classifications and detect potential outliers. An elastic net feature selection method was used to first identify discriminatory features within the datasets. Finally, the selected, cross-validated, features were input into an orthogonal partial least squares discriminatory analysis (OPLS-DA) to generate diagnostic mathematical models. Analysis was performed on each biofluid and dataset independently (proteomics and metabolomics) and all combined permutations in a combined-omics approach. All OPLS-DA models were optimized by internal 7-fold cross-validation. The quality of classification was assessed using a 10-fold external cross-validation scheme with 1000 repetitions in total (to correct for unequal class sizes). This validation scheme involves multiple iterations of splitting the data into training and testing sets. The training data is used to estimate the model parameters and learn the underlying discriminatory patterns between the groups under consideration, whereas the independent test set is employed to assess the accuracy and generalizability of the trained models in the ensemble. We quantified the response of the ensemble of models by calculating the accuracy, sensitivity, and specificity of each model from the predicted classifications of the external, independent test set (i.e. which is not used in model building). It is important to appreciate that the classifier (OPLS-DA) was blinded to the test set during the process of model training. This validation scheme tends to avoid over-fitting and helps assess the generalizability of the model to previously unseen datasets. For an exhaustive discussion on validation see Arlot and Celisse (2010) [Statistics surveys 4:40-79]. These values were compared with those of a null distribution (obtained from randomly permuting the classifications) using the two-sided Kolmogorov-Smirnov test (significant if p-value 0.05 or less). Discriminators were identified by calculating the variable importance (VIP) score (cut-off of 1.5). The fold changes of these variables were further investigated by analysis-of-variance (ANOVA) followed by Tukey's honest significant difference (HSD) post-hoc test. The p-values obtained were then corrected for multiple comparisons using the Bonferroni correction.

### Example 1

Metabolomics analysis was able to diagnose CDMS and CIS with accuracies of 71±4% and 66±2% respectively. Interestingly, removal of CIS patients who tested negative for OCBs resulted in an improved accuracy of 68±3%.

Table 1 shows top protein biomarkers identified by feature selection and multivariate analysis and ranked by importance in the multivariate model. Univariate p-value <0.05, 0.01, 0.001 represented by *,**,*** respectively. Univariate p-value <0.05, 0.01, 0.001 following Bonferonni correction for multiple comparisons represented by ‡, ‡‡, ‡‡‡ respectively:

| | | Uniprot # | | | | |
|---|---|---|---|---|---|---|
| | | (Entry | | | | |
| | | Version & | | | | |
| | | Sequence | | | | |
| | | Version | | | | |
| | | as per the | | Early | Non- | Fold Change |
| | | Sequence | | converter | converter | (univariate |
| Rank | Target | Listing) | Protein Name | mean ± sd | mean ± sd | significance) |
| 1 | RSK- | O75582 | Ribosomal | 24.3 ± 3.7 | 27.9 ± 2.5 | 0.87 (‡) |
| | like | | protein S6 | | | |
| | protein | | kinase alpha-5 | | | |
| | kinase | | | | | |
| 2 | XRCC1 | P18887 | DNA repair | 68.3 ± 2.4 | 72.1 ± 3.6 | 0.95 (***) |
| | | | protein XRCC1 | | | |
| 3 | BACH | O00154 | Cytosolic acyl | 131.3 ± 22.3 | 110.3 ± 25.6 | 1.19 (**) |
| | | | coenzyme A | | | |
| | | | thioester | | | |
| | | | hydrolase | | | |
| 4 | BMX | P51813 | Cytoplasmic | 75.2 ± 4.4 | 79.5 ± 4.6 | 0.95 (***) |
| | | | tyrosine- | | | |
| | | | protein kinase | | | |
| | | | BMX | | | |
| 5 | cathepsin | P43235 | Cathepsin K | 217.9 ± 60.3 | 285.6 ± 69.7 | 0.76 (***) |
| | K | | | | | |
| 6 | Tropomyosin | P06753 | Tropomyosin | 139.3 ± 6.8 | 153.5 ± 17.3 | 0.91 (***) |
| | alpha-3 | | alpha-3 chain | | | |
| | chain | | | | | |
| 7 | ARHG2 | Q92974 | Rho guanine | 74.7 ± 4.6 | 69.9 ± 6.5 | 1.07 (**) |
| | | | nucleotide | | | |
| | | | exchange | | | |
| | | | factor 2 | | | |
| 8 | PKHA1 | Q9HB21 | Pleckstrin | 38.3 ± 3.3 | 41.4 ± 3 | 0.92 (***) |
| | | | homology | | | |
| | | | domain-containing | | | |
| | | | family A | | | |
| | | | member 1 | | | |
| 9 | EFHA1 | Q8IYU8 | Calcium | 326.5 ± 46.7 | 376.3 ± 50.7 | 0.87 (***) |
| | | | uptake protein | | | |
| | | | 2, | | | |
| | | | mitochondrial | | | |
| 10 | RN165 | Q6ZSG1 | RING finger | 337.8 ± 142.2 | 215.4 ± 85.2 | 1.57 (***) |
| | | | protein 165 | | | |
| 11 | NKp46 | O76036 | Natural | 229.9 ± 26.4 | 206.8 ± 42.4 | 1.11 (*) |
| | | | cytotoxicity | | | |
| | | | triggering | | | |
| | | | receptor 1 | | | |
| 12 | AP1G2 | O75843 | AP-1 complex | 646.4 ± 83 | 769 ± 161.1 | 0.84 (***) |
| | | | subunit | | | |
| | | | gamma-like 2 | | | |
| 13 | LTB4DH | Q14914 | Prostaglandin | 149.5 ± 32.6 | 194.7 ± 55.3 | 0.77 (***) |
| | | | reductase 1 | | | |
| 14 | IL-5 Ra | Q01344 | Interleukin-5 | 108.7 ± 21.8 | 91.1 ± 14.3 | 1.19 (***) |
| | | | receptor | | | |
| | | | subunit alpha | | | |
| 15 | TSSK2 | Q96PF2 | Testis-specific | 79 ± 4.3 | 83.7 ± 5.5 | 0.94 (***) |
| | | | serine/threonine- | | | |
| | | | protein | | | |
| | | | kinase 2 | | | |
| 16 | BLK | P51451 | Tyrosine- | 54.7 ± 4.2 | 59.2 ± 5.4 | 0.92 (***) |
| | | | protein kinase | | | |
| | | | BLK | | | |
| 17 | Lymphotoxin | P01374, | Lymphotoxin | 406.3 ± 176.7 | 266.8 ± 116.8 | 1.52 (***) |
| | a2/b1 | Q06643 | alpha2:beta1 | | | |
| 18 | MAVS | Q7Z434 | Mitochondrial | 79.2 ± 17.2 | 63.6 ± 13.8 | 1.25 (***) |
| | | | antiviral- | | | |
| | | | signaling | | | |
| | | | protein | | | |
| 19 | GCH1 | P30793 | GTP | 91.3 ± 8.2 | 83.6 ± 8.1 | 1.09 (***) |
| | | | cyclohydrolase | | | |
| | | | 1 | | | |
| 20 | NovH | P48745 | Protein NOV | 4128.4 ± 1423.4 | 5498 ± 1798.2 | 0.75 (**) |
| | | | homolog | | | |
| 21 | MZT1 | Q08AG7 | Mitotic-spindle | 69 ± 3.2 | 72 ± 4.4 | 0.96 (**) |
| | | | organizing | | | |
| | | | protein 1 | | | |
| 22 | MCF2L | O15068 | Guanine | 103 ± 8.7 | 96.2 ± 7.3 | 1.07 (**) |
| | | | nucleotide | | | |
| | | | exchange | | | |
| | | | factor DBS | | | |
| 23 | VCAM-1 | P19320 | Vascular cell | 228.1 ± 56.4 | 181.9 ± 46.6 | 1.25 (***) |
| | | | adhesion | | | |
| | | | protein 1 | | | |
| 24 | CBR1 | P16152 | Carbonyl | 1765.1 ± 408.5 | 2220.6 ± 628 | 0.79 (**) |
| | | | reductase | | | |
| | | | [NADPH] 1 | | | |
| 25 | LCN10 | Q6JVE6 | Epididymal- | 59.4 ± 5.6 | 55.2 ± 3.5 | 1.08 (***) |
| | | | specific | | | |
| | | | lipocalin-10 | | | |
| 26 | URB | Q76M96 | Coiled-coil | 295 ± 42 | 337.9 ± 52.4 | 0.87 (**) |
| | | | domain- | | | |
| | | | containing | | | |
| | | | protein 80 | | | |
| 27 | GRAN | P28676 | Grancalcin | 49 ± 2.3 | 52.7 ± 6 | 0.93 (**) |
| 28 | GLTL1 | Q8N428 | Polypeptide N- | 3933 ± 761.3 | 4858.3 ± 1019.3 | 0.81 (***) |
| | | | acetylgalactos | | | |
| | | | aminyltransferase | | | |
| | | | 16 | | | |
| 29 | Factor H | P08603 | Complement | 4372.3 ± 473.8 | 4797.6 ± 519.4 | 0.91 (**) |
| | | | factor H | | | |
| 30 | IL32 | P24001 | Interleukin-32 | 64.2 ± 2.6 | 68.3 ± 5.3 | 0.94 (***) |
| 31 | c-Raf | P04049 | RAF proto- | 192.8 ± 56.6 | 217.4 ± 46 | 0.89 (*) |
| | | | oncogene | | | |
| | | | serine/threonine- | | | |
| | | | protein kinase | | | |
| 32 | GLCE | O94923 | D-glucuronyl | 426.4 ± 66.3 | 479.6 ± 66.8 | 0.89 (**) |
| | | | C5-epimerase | | | |
| 33 | ADRM1 | Q16186 | Proteasomal | 61.7 ± 3.8 | 66.4 ± 6.3 | 0.93(**) |
| | | | ubiquitin | | | |
| | | | receptor | | | |
| | | | ADRM1 | | | |
| 34 | NR1D2 | Q14995 | Nuclear | 51.2 ± 2.1 | 53.8 ± 4.5 | 0.95 (**) |
| | | | receptor | | | |
| | | | subfamily 1 | | | |
| | | | group D | | | |
| | | | member 2 | | | |
| 35 | CRBB2 | P43320 | Beta-crystallin | 145.2 ± 15.9 | 151.1 ± 63 | 0.96 (ns) |
| | | | B2 | | | |
| 36 | ZNF41 | P51814 | Zinc finger | 83.6 ± 7.3 | 90.7 ± 31.6 | 0.92 (ns) |
| | | | protein 41 | | | |
| 37 | ELK1 | P19419 | ETS domain- | 154 ± 32 | 146.5 ± 17.6 | 1.05 (ns) |
| | | | containing | | | |
| | | | protein Elk-1 | | | |
| 38 | GUC1A | P43080 | Guanylyl | 77.8 ± 5.6 | 78.5 ± 6.1 | 0.99 (ns) |
| | | | cyclase-activating | | | |
| | | | protein 1 | | | |
| 39 | IRF1 | P10914 | Interferon | 63.2 ± 3.2 | 63.7 ± 2.9 | 0.99 (ns) |
| | | | regulatory | | | |
| | | | factor 1 | | | |
| 40 | BECN1 | Q14457 | Beclin-1 | 81.8 ± 13.9 | 80.1 ± 6 | 1.02 (ns) |
| 41 | OCRL | Q01968 | Inositol | 222.6 ± 14 | 226.5 ± 12.1 | 0.98 (ns) |
| | | | polyphosphate | | | |
| | | | 5-phosphatase | | | |
| | | | OCRL-1 | | | |
| 42 | DYLT1 | P63172 | Dynein light | 114 ± 15.2 | 105.4 ± 12.3 | 1.08(*) |
| | | | chain Tctex- | | | |
| | | | type 1 | | | |
| 43 | TSP2 | P35442 | Thrombospondin- | 36082.5 ± 7662.1 | 42065.7 ± 6172.7 | 0.86 (**) |
| | | | 2 | | | |
| 44 | TARC | Q92583 | C-C motif | 117.4 ± 62.8 | 76.8 ± 19.3 | 1.53 (***) |
| | | | chemokine 17 | | | |
| 45 | DRGX | A6NNA5 | Dorsal root | 92.4 ± 11 | 102.4 ± 15 | 0.9 (**) |
| | | | ganglia | | | |
| | | | homeobox | | | |
| | | | protein | | | |
| 46 | PENK | P01210 | Proenkephalin- | 523.1 ± 139.9 | 800.7 ± 576.7 | 0.65 (*) |
| | | | A | | | |
| 47 | LY9 | Q9HBG7 | T-lymphocyte | 190.1 ± 57.7 | 146.3 ± 36.1 | 1.3 (***) |
| | | | surface | | | |
| | | | antigen Ly-9 | | | |
| 48 | MUSK | O15146 | Muscle, | 131.3 ± 23.9 | 149.6 ± 29.1 | 0.88 (**) |
| | | | skeletal | | | |
| | | | receptor | | | |
| | | | tyrosine- | | | |
| | | | protein kinase | | | |
| 49 | MZF1 | P28698 | Myeloid zinc | 190.3 ± 28.7 | 170.1 ± 25.7 | 1.12 (**) |
| | | | finger 1 | | | |
| 50 | DGCR6 | Q14129 | Protein | 100.2 ± 9.8 | 93 ± 7.3 | 1.08 (**) |
| | | | DGCR6 | | | |
| 51 | EDAR | Q9UNE0 | Tumor | 384.6 ± 50.9 | 449.6 ± 80.4 | 0.86 (***) |
| | | | necrosis factor | | | |
| | | | receptor | | | |
| | | | superfamily | | | |
| | | | member EDAR | | | |
| 52 | PCDA7 | Q9UN72 | Protocadherin | 436.5 ± 89.9 | 530.4 ± 130.1 | 0.82 (**) |
| | | | alpha-7 | | | |
| 53 | FCGR1 | P12314 | High affinity | | | |
| | | | immunoglobulin | | | |
| | | | gamma Fc | 162.3 ± 45.9 | 202.9 ± 58 | 0.8 (**) |
| | | | receptor I | | | |
| 54 | CD40 | P29965 | CD40 ligand | 53.8 ± 4.7 | 57.2 ± 5.6 | 0.94 (*) |
| | ligand, | | | | | |
| | soluble | | | | | |
| 55 | DKK2 | Q9UBU2 | Dickkopf- | 1298 ± 452 | 1653 ± 497.3 | 0.79 (**) |
| | | | related protein | | | |
| | | | 2 | | | |
| 56 | Gro-a | P09341 | Growth- | 1163.1 ± 366.9 | 891.8 ± 262.7 | 1.3 (***) |
| | | | regulated | | | |
| | | | alpha protein | | | |
| 57 | TIMP-2 | P16035 | Metalloproteinase | 1662.3 ± 125.3 | 1761.2 ± 163.9 | 0.94 (**) |
| | | | inhibitor 2 | | | |
| 58 | BOC | Q9BWV1 | Brother of | 4235.3 ± 813.8 | 5147.9 ± 1057.1 | 0.82 (***) |
| | | | CDO | | | |
| 59 | F175B | Q15018 | BRISC | 31.7 ± 3.6 | 34.1 ± 3.8 | 0.93 (*) |
| | | | complex | | | |
| | | | subunit Abro1 | | | |
| 60 | TXNDC4 | Q9BS26 | Endoplasmic | 494.1 ± 91.5 | 549.8 ± 95.4 | 0.9 (*) |
| | | | reticulum | | | |
| | | | resident | | | |
| | | | protein 44 | | | |
| 61 | CLFB_STAAE | O86476 | Clumping | 193.8 ± 45.9 | 171.3 ± 37.8 | 1.13 (*) |
| | | | factor B | | | |
| 62 | MMP-13 | P45452 | Collagenase 3 | 98.7 ± 10.3 | 111.7 ± 23.6 | 0.88 (**) |
| 63 | Prokineticin-2 | Q9H023 | Prokineticin-2 | 133 ± 24.7 | 153.9 ± 31.2 | 0.86 (**) |
| 64 | PPIL2 | Q13356 | Peptidyl-prolyl | 97.6 ± 10.3 | 90.8 ± 9 | 1.08 (**) |
| | | | cis-trans | | | |
| | | | isomerase-like | | | |
| | | | 2 | | | |
| 65 | IL-22BP | Q969J5 | Interleukin-22 | 196.5 ± 43.3 | 167 ± 14.4 | 1.18 (***) |
| | | | receptor | | | |
| | | | subunit alpha- | | | |
| | | | 2 | | | |
| 66 | SGCB | Q16585 | Beta- | 106.4 ± 17.8 | 92.1 ± 15.2 | 1.16 (**) |
| | | | sarcoglycan | | | |
| 67 | GPNMB | Q14956 | Transmembrane | 857 ± 285.9 | 660.4 ± 188.9 | 1.3 (**) |
| | | | glycoprotein | | | |
| | | | NMB | | | |
| 68 | OPG | O00300 | Tumor | 9157.2 ± 1882 | 11251.8 ± 2706.5 | 0.81 (**) |
| | | | necrosis factor | | | |
| | | | receptor | | | |
| | | | superfamily | | | |
| | | | member 11B | | | |
| 69 | MFAP4 | P55083 | Microfibril- | | | |
| | | | associated | 9029 ± 3146.1 | 12137.1 ± 3213.7 | 0.74 (***) |
| | | | glycoprotein 4 | | | |
| 70 | C06A2 | P12110 | Collagen | 29.7 ± 4 | 27.3 ± 3.1 | 1.09 (**) |
| | | | alpha-2(VI) | | | |
| | | | chain | | | |
| 71 | ELL2 | O00472 | RNA | 261.7 ± 17.3 | 277.2 ± 21 | 0.94 (**) |
| | | | polymerase II | | | |
| | | | elongation | | | |
| | | | factor ELL2 | | | |
| 72 | K0494 | O75071 | EF-hand | 507.5 ± 176.9 | 806.9 ± 484.6 | 0.63 (**) |
| | | | calcium-binding | | | |
| | | | domain- | | | |
| | | | containing | | | |
| | | | protein 14 | | | |
| 73 | CV032 | Q9H4I9 | Essential MCU | 23.1 ± 2.3 | 25.1 ± 2.3 | 0.92 (**) |
| | | | regulator, | | | |
| | | | mitochondrial | | | |
| 74 | Karyopherin- | P52292 | Importin | 47.9 ± 3.8 | 51.6 ± 4.7 | 0.93 (**) |
| | a2 | | subunit alpha- | | | |
| | | | 1 | | | |
| 75 | LRRC3 | Q9BY71 | Leucine-rich | 122.1 ± 9.8 | 131.3 ± 24.1 | 0.93 (*) |
| | | | repeat- | | | |
| | | | containing | | | |
| | | | protein 3 | | | |
| 76 | VSIG2 | Q96IQ7 | V-set and | 240.2 ± 25.6 | 264 ± 33.1 | 0.91 (**) |
| | | | immunoglobulin | | | |
| | | | domain- | | | |
| | | | containing | | | |
| | | | protein 2 | | | |
| 77 | ISK13 | Q1W4C9 | Serine | 60.9 ± 3.6 | 67.7 ± 20.1 | 0.9 (ns) |
| | | | protease | | | |
| | | | inhibitor Kazal- | | | |
| | | | type 13 | | | |
| 78 | IGFBP-1 | P08833 | Insulin-like | 59.2 ± 10.2 | 65.5 ± 19.5 | 0.9 (ns) |
| | | | growth factor- | | | |
| | | | binding protein | | | |
| | | | 1 | | | |
| 79 | DB123 | Q8N688 | Beta-defensin | 84.4 ± 24.7 | 66.2 ± 14 | 1.28 (***) |
| | | | 123 | | | |
| 80 | SPAT9 | Q9BWV2 | Spermatogene | 209.4 ± 39.8 | 233.8 ± 37.5 | 0.9 (*) |
| | | | sis-associated | | | |
| | | | protein 9 | | | |
| 81 | hnRNPK | P61978 | Heterogeneous | 54.7 ± 2.2 | 56.5 ± 3.1 | 0.97 (*) |
| | | | nuclear | | | |
| | | | ribonucleoprotein | | | |
| | | | K | | | |
| 82 | DBNL | Q9UJU6 | Drebrin-like | 348.7 ± 49.4 | 390.4 ± 67.4 | 0.89 (**) |
| | | | protein | | | |
| 83 | DHH | O43323 | Desert | 575.8 ± 108.8 | 715.6 ± 199.6 | 0.8 (**) |
| | | | hedgehog | | | |
| | | | protein N- | | | |
| | | | product | | | |
| 84 | Activin | P08476 | Inhibin beta A | 411.7 ± 107.4 | 505.8 ± 117.1 | 0.81 (**) |
| | AB | P09529 | chain:Inhibin | | | |
| | | | beta B chain | | | |
| | | | heterodimer | | | |
| 85 | TIG2 | Q99969 | Retinoic acid | 4348.9 ± 847.7 | 4858.7 ± 808.3 | 0.9 (*) |
| | | | receptor | | | |
| | | | responder | | | |
| | | | protein 2 | | | |
| 86 | LSHR | P22888 | Lutropin- | 47.4 ± 2.5 | 49.6 ± 6.7 | 0.96 (ns) |
| | | | choriogonadotropic | | | |
| | | | hormone | | | |
| | | | receptor | | | |
| 87 | TSP4 | P35443 | Thrombospondin- | 253.5 ± 49.2 | 285.8 ± 57 | 0.89 (*) |
| | | | 4 | | | |
| 88 | GFAP | P14136 | Glial fibrillary | 16249.3 ± 16945.5 | 10561.9 ± 15391.6 | 1.54 (ns) |
| | | | acidic protein | | | |
| 89 | MILR1 | Q7Z6M3 | Allergin-1 | 100.6 ± 7.4 | 106.4 ± 6.2 | 0.95 (**) |

Table 2 shows CSF NMR metabolomics hits identified. Rank in combined 'omics mode. Mean±standard deviation relative spectral intensity. Fold change of early converters relative to non-converters. Univariate p-value <0.05, 0.01, 0.001 represented by *,**,*** respectively. Univariate p-value <0.05, 0.01, 0.001 following Bonferonni correction for multiple comparisons represented by ‡, ‡‡, ‡‡‡ respectively.

| | Metabolite | | | | | |
|---|---|---|---|---|---|---|
| | (multiplicity | | | | Fold | |
| Chemical | of major | | Early | non- | Change | |
| Shift | selected | | converter | converter | (univariate | |
| (ppm) | peak) | Rank | mean ± sd | mean ± sd | significance) | |
| 3.03-3.05 | creatinine (s), | 1 | 0.005 ± 0.0007 | 0.005 ± 0.0005 | 0.96 | (ns) |
| | creatine (s) | | | | | |
| 0.95-0.97 | isoleucine (t) | 2 | 0.000454669 | 0.002 ± 0.0004 | 0.93 | (ns) |
| 0.97-0.99 | leucine (d) | 3 | 0.000408751 | 0.002 ± 0.0004 | 0.94 | (ns) |
| 3.93-3.95 | Betaine (s) | 4 | 0.005 ± 0.0005 | 0.005 ± 0.0005 | 0.95 | (*) |
| 8.45-8.47 | formate (s) | 5 | 0.011 ± 0.0012 | 0.011 ± 0.0013 | 1.04 | (ns) |
| 3.03-3.05, | myo- | 6 | 0.004 ± 0.0006 | 0.005 ± 0.0005 | 0.93 | (*) |
| 4.05-4.07 | inositol (t) | | | | | |
| 3.39-3.91 | glucose | 7 | 0.046 ± 0.0037 | 0.044 ± 0.0032 | 1.05 | (*) |
| | (multiple in | | | | | |
| | this region) | | | | | |
| 2.13-2.15 | glutamine (m) | 8 | 0.013 ± 0.0013 | 0.013 ± 0.0016 | 0.99 | (ns) |
| 1.31-1.35 | lactate (d) | 9 | 0.047 ± 0.0049 | 0.046 ± 0.0045 | 1.03 | (ns) |

Key: s-singlet; d-doublet; t-triplet; m-multiplet.

Table 3 shows serum NMR metabolomics hits identified. Rank in combined 'omics mode. Mean±standard deviation relative spectral intensity. Fold change of early converters relative to non-converters. Univariate p-value <0.05, 0.01, 0.001 represented by *,**,*** respectively. Univariate p-value <0.05, 0.01, 0.001 following Bonferonni correction for multiple comparisons represented by ‡, ‡‡, ‡‡‡ respectively.

| | | | | | Fold |
|---|---|---|---|---|---|
| Chemical | | | Early | non- | Change |
| Shift | | | converter | converter | (univariate |
| (ppm) | Metabolite | Rank | mean ± sd | mean ± sd | significance) |
| 1.22-1.28 | mobile | 1 | 0.018 ± 0.003 | 0.017 ± 0.0025 | 1.07 (ns) |
| | lipoprotein | | | | |
| | (—CH2—)n | | | | |
| | resonances | | | | |
| | (VLDL and LDL) | | | | |
| 3.4-3.90 | glucose | 2 | 0.015 ± 0.002 | 0.015 ± 0.0026 | 0.96 (ns) |
| 0.80-0.88 | Mobile lipoprotein | 3 | 0.014 ± 0.0019 | 0.013 ± 0.002 | 1.03 (ns) |
| | —CH3 resonances | | | | |
| | (HDL and LDL) | | | | |
| 3.20-3.22 | mobile- | 4 | 0.02 ± 0.002 | 0.019 ± 0.0023 | 1.01 (ns) |
| | N(CH3)3/ | | | | |
| | free choline | | | | |
| 1.18-1.22 | 3-hydroxybutyrate | 5 | 0.004 ± 0.0009 | 0.005 ± 0.0016 | 0.96 (ns) |
| 2.04-2.06 | NAC1/═CH— | 6 | 0.033 ± 0.0045 | 0.032 ± 0.0048 | 1.01 (ns) |
| | CH2—CH2— | | | | |

Table 4 shows Clinical Chemistry parameters included in models. Rank in combined 'omics mode. Mean±standard deviation. Fold change of early converters relative to non-converters. Univariate p-value <0.05, 0.01, 0.001 represented by *,**,*** respectively. Univariate p-value <0.05, 0.01, 0.001 following Bonferonni correction for multiple comparisons represented by ‡, ‡‡, ‡‡‡ respectively.

| Other | | | | | |
|---|---|---|---|---|---|
| measures | | | | Fold | |
| included in | | Early | non- | Change | |
| combined | | converter | converter | (univariate | |
| omics | Rank | mean ± sd | mean ± sd | significance) | |
| OCB status | 1 | NA | NA | NA | |
| CSF leukocytes | 2 | 10.9 ± 9.1 | 4.6 ± 4.7 | 2.36 | (***) |
| mononuclear | 3 | 10.7 ± 8.8 | 7.2 ± 17.4 | 1.5 | (ns) |
| polynuclear | 4 | 0.3 ± 0.7 | 0.2 ± 0.6 | 1.42 | (ns) |
| CSF/serum | 5 | 4.9 ± 2 | 5.2 ± 1.7 | 0.94 | (ns) |
| albumin ratio | | | | | |
| CSF total | 6 | 367.7 ± 122.5 | 373.3 ± 95.7 | 0.98 | (ns) |
| protein | | | | | |

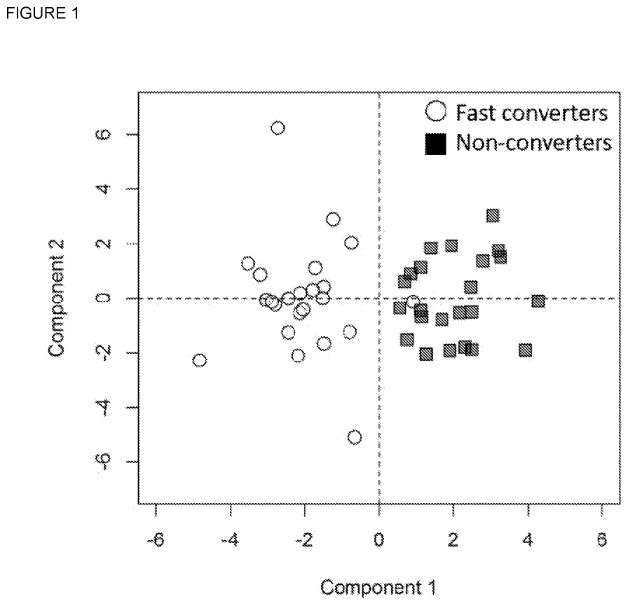

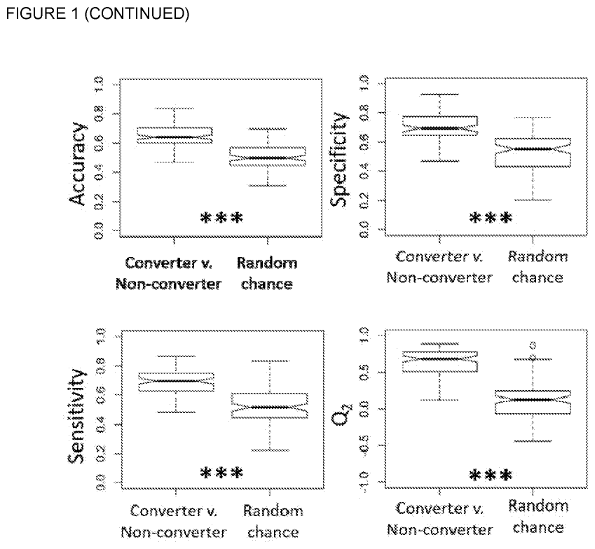

FIG. 1 shows representative OPLS-DA scores plot illustrating excellent discrimination between the non-converter (black square) and early converter (white circle) CIS patients using CSF proteomics features combined with CSF NMR metabolomic features. The accuracy, sensitivity, specificity, and cumulative Q² of the ensemble of 1000 early converter V. non-converter models, as determined by classification of an independent test set, is significantly greater than that of random data confirming that the models are well-validated and significant. Kolmogorov-Smirnov test p-values <0.001 are represented by ***.

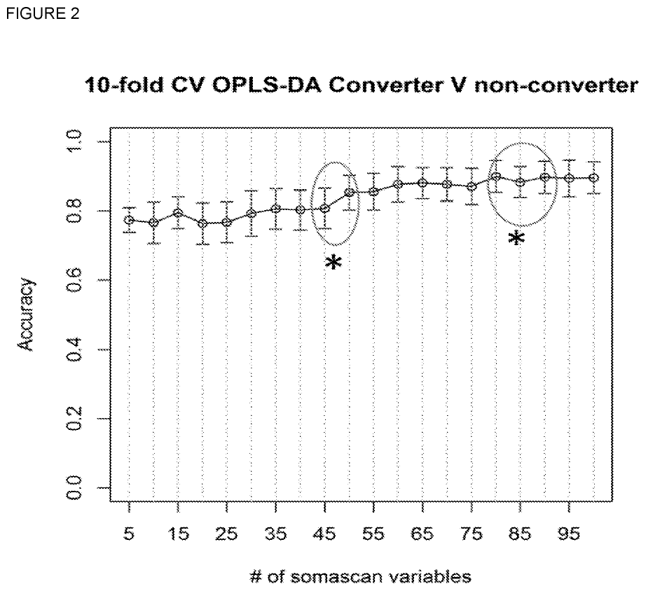

FIG. 2 shows accuracy of 10-fold cross-validated OPLS-DA models at discriminating fast and slow converters with increasing numbers of protein features. Diagnostic accuracy increases as the number of protein features included in the models increases. An accuracy of 77% is achieved with only 5 protein hits. A significant increase in accuracy to 85% is observed when the number of features is increased to 55. A maximum accuracy of 90% is observed with between 80-90 features.

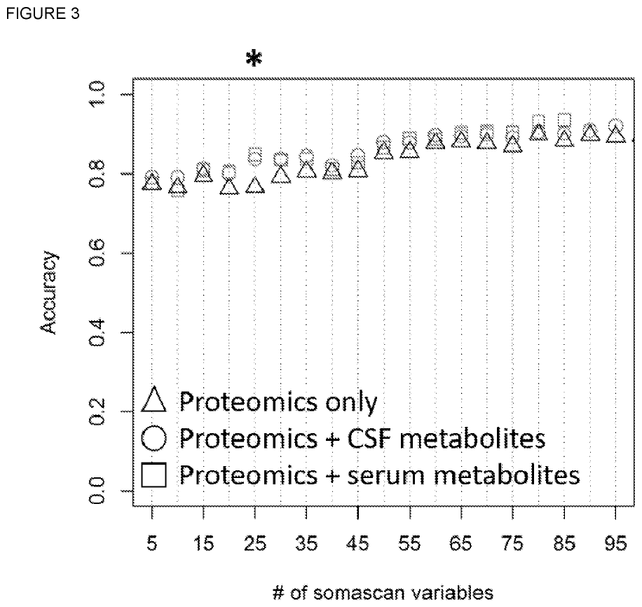

FIG. 3 shows accuracy of 10-fold cross-validated OPLS-DA models at discriminating fast and slow converters with increasing numbers of protein features (triangle), including CSF metabolite hits (circle) or serum metabolites hits (square). Addition of the top metabolites hits from either CSF or serum results in an increased accuracy of 85% using only 25 protein markers, in contrast to the 55 protein variables required to achieve the same accuracy using the proteomics analysis alone.

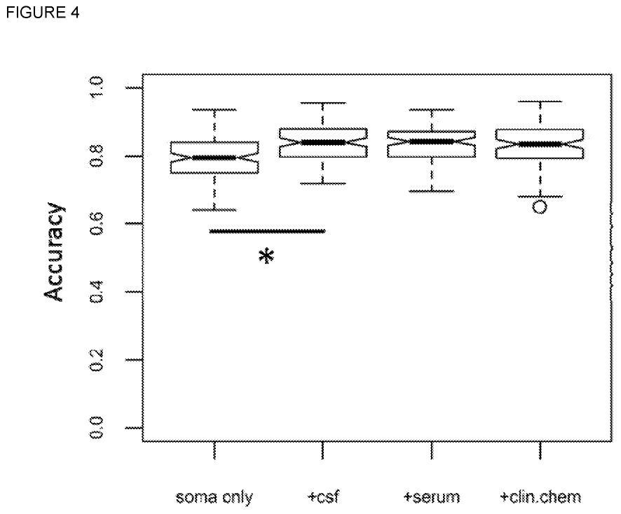

FIG. 4 shows accuracy of 10-fold cross-validated OPLS-DA models discriminating between fast and slow converters using proteomics features only (soma only), proteomics and CSF metabolomics features (+csf), proteomics and serum metabolomics features (+serum), and proteomics plus clinical chemistry parameters (+clin.chem). Addition of either CSF, serum, or clinical chemistry metabolite features to the identified protein biomarkers improves the diagnostic accuracy, highlighting the advantage of using a combined-omics methodology.

These results indicate that metabolomics and proteomics analyses could not only be used in diagnosis of CDMS but could also be used as a prognostic test to identify CIS patients at high risk of a second clinical attack within 4 years of onset.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for determining conversion of a subject from clinically isolated syndrome (CIS) to clinically definite multiple sclerosis (CDMS), the method comprising:
a. providing a sample obtained from the subject, wherein the subject has, or is suspected of having, CIS;
b. measuring a concentration of:
i. one or more polypeptides in the sample; and/or
ii. one or more metabolites in the sample;
c. comparing the measured concentration with the concentration of the same one or more polypeptides and/or metabolites, respectively, in a reference standard; and
d. determining that the subject will convert from CIS to CDMS based on the comparison, or determining that the subject will not convert from CIS to CDMS based on the comparison.

2. The method according to claim 1, wherein the reference standard is a non-convertor reference standard from a subject who has CIS.

3. The method according to claim 1, wherein the reference standard is a convertor reference standard from a subject who has CDMS.

4. The method according to any one of the preceding claims, wherein the method determines whether or not a subject will convert from CIS to CDMS within a period of 10 years or 5 years of CIS or preferably within a period of 4 years of CIS.

5. The method according to any one of the preceding claims, wherein step b. comprises measuring the concentration of one or more metabolites and one or more polypeptides in the sample.

6. The method according to any one of the preceding claims, wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1.

7. The method according to any one of the preceding claims, wherein the one or more metabolites are selected from: creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate.

8. The method according to any one of the preceding claims, wherein the one or more metabolites are:
one or more cerebrospinal fluid metabolites selected from: creatinine, creatine, isoleucine, leucine, betaine, formate, myo-inositol, glucose, glutamine, and lactate; and/or
one or more serum metabolites selected from: mobile lipoprotein (—CH2-)n resonances (VLDL and LDL), glucose, mobile lipoprotein —CH3 resonances (HDL and LDL), mobile —N(CH3)3/free choline, 3-hydroxybutyrate, and NAC1/=CH—CH2-CH2-.

9. The method according to any one of the preceding claims, wherein step d. comprises:
determining that the subject will convert from CIS to CDMS when:
i. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is increased when compared to the reference standard when the reference standard is a non-convertor reference standard; or
ii. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is decreased when compared to the reference standard when the reference standard is a non-convertor reference standard; or
iii. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is increased or the same when compared to the reference standard when the reference standard is a convertor reference standard; or
iv. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is decreased or the same when compared to the reference standard when the reference standard is a convertor reference standard; or
determining that the subject will not convert from CIS to CDMS when:
i. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is decreased or the same when compared to the reference standard when the reference standard is a non-convertor reference standard; or
ii. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is increased or the same when compared to the reference standard when the reference standard is a non-convertor reference standard; or
iii. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is decreased when compared to the reference standard when the reference standard is a convertor reference standard; or
iv. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is increased when compared to the reference standard when the reference standard is a convertor reference standard.

10. The method according to any one of the preceding claims, wherein step d. comprises:
determining that the subject will convert from CIS to CDMS when:
i. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the measured concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
iv. the measured concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard; or
determining that the subject will not convert from CIS to CDMS when:
i. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
ii. the measured concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
iii. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
iv. the measured concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard.

11. The method according to any one of the preceding claims, wherein step d. comprises:
i. determining that the subject will convert from CIS to CDMS and identifying the subject as a fast converter based on the comparison; or
ii. determining that the subject will not convert from CIS to CDMS and identifying the subject as a slow converter or a non-convertor based on the comparison.

12. The method according to any one of the preceding claims, further comprising measuring leukocyte concentration, mononuclear cell concentration, polynuclear cell concentration, serum albumin ratio and total protein concentration in a sample obtained from the subject.

13. The method according to claim 12, wherein the concentration of leukocytes, mononuclear and/or polynuclear cells in a sample obtained from the subject is:
i. decreased or the same when compared to a non-convertor reference standard and determines that a subject will not convert from CIS to CDMS;
ii. decreased when compared to a convertor reference standard and determines that a subject will not convert from CIS to CDMS;
iii. increased or the same when compared to a convertor reference standard and determines that a subject will convert from CIS to CDMS; or
iv. increased when compared to a non-convertor reference standard and determines that a subject will convert from CIS to CDMS.

14. The method according to claim 12 or 13, wherein the serum/albumin ratio and/or total protein concentration in a sample obtained from the subject is:
i. decreased or the same when compared to a convertor reference standard and determines that a subject will convert from CIS to CDMS;
ii. decreased when compared to a non-convertor reference standard and determines that a subject will convert from CIS to CDMS;
iii. increased or the same when compared to a non-convertor reference standard and determines that a subject will not convert from CIS to CDMS; or
iv. increased when compared to a convertor reference standard and determines that a subject will not convert from CIS to CDMS.

15. A method for diagnosing multiple sclerosis (MS), the method comprising:
a. providing a sample obtained from a subject;
b. measuring a concentration of:
i. one or more polypeptides in the sample, wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
ii. one or more metabolites in the sample, wherein the one or more metabolites are selected from: creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the measured concentration with the concentration of the same one or more polypeptides and/or metabolites, respectively, in a reference standard; and
d. diagnosing MS, or not diagnosing MS based on the comparison.

16. The method according to claim 15, wherein the reference standard is a non-convertor reference standard from a subject who has CIS.

17. The method according to claim 15, wherein the reference standard is a convertor reference standard from a subject who has CDMS.

18. The method according to any one of claims 15-17, wherein the method is for diagnosing clinically definite multiple sclerosis (CDMS), and wherein step d. comprises diagnosing CDMS based on the comparison, or not diagnosing CDMS or diagnosing CIS based on the comparison.

19. The method according to any one of claims 15-18, wherein step d. comprises:
diagnosing MS when:
i. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is increased when compared to the reference standard when the reference standard is a non-convertor reference standard; or
ii. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is decreased when compared to the reference standard when the reference standard is a non-convertor reference standard; or
iii. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is increased or the same when compared to the reference standard when the reference standard is a convertor reference standard; or
iv. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4m and Allergin-1 is decreased or the same when compared to the reference standard when the reference standard is a convertor reference standard; or
not diagnosing MS when:
i. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is decreased or the same when compared to the reference standard when the reference standard is a non-convertor reference standard; or
ii. the measured concentration of one or more polypeptides selected from:
Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is increased or the same when compared to the reference standard when the reference standard is a non-convertor reference standard; or
iii. the measured concentration of one or more polypeptides selected from:
Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is decreased when compared to the reference standard when the reference standard is a convertor reference standard; or
iv. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is increased when compared to the reference standard when the reference standard is a convertor reference standard.

20. The method according to any one of claims 15-19, wherein step d. comprises:
diagnosing MS when:
i. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the measured concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
iv. the measured concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard; or
not diagnosing MS when:
i. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
ii. the measured concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
iii. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
iv. the measured concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard.

21. The method according to any one of claims 15-20, further comprising measuring leukocyte concentration, mononuclear cell concentration, polynuclear cell concentration, serum albumin ratio and total protein concentration in a sample obtained from the subject.

22. The method according to claim 21, wherein the concentration of leukocytes, mononuclear and/or polynuclear cells in a sample obtained from the subject is:
i. decreased or the same when compared to a non-convertor reference standard and does not diagnose a subject with MS;
ii. decreased when compared to a convertor reference standard and does not diagnose a subject with MS;
iii. increased or the same when compared to a convertor reference standard and diagnoses a subject with MS; or
iv. increased when compared to a non-convertor reference standard and diagnoses a subject with MS.

23. The method according to claim 21 or 22, wherein the serum/albumin ratio and/or total protein concentration in a sample obtained from the subject is:
i. decreased or the same when compared to a convertor reference standard and diagnoses a subject with MS;
ii. decreased when compared to a non-convertor reference standard and diagnoses a subject with MS;
iii. increased or the same when compared to a non-convertor reference standard and does not diagnose a subject with MS;
iv. increased when compared to a convertor reference standard and does not diagnose a subject with MS.

24. A method for determining prognosis of multiple sclerosis (MS), the method comprising:
a. providing a sample obtained from a subject;
b. measuring a concentration of:
i. one or more polypeptides in the sample, wherein the one or more polypeptides are selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
ii. one or more metabolites in the sample, wherein the one or more metabolites are selected from: creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the concentration with the concentration of the same one or more polypeptides and/or metabolites, respectively, in a reference standard; and
d. determining that the subject's prognosis is poor based on the comparison or determining that the subject's prognosis is good based on the comparison.

25. The method according to claim 24, wherein the reference standard is a non-convertor reference standard from a subject who has CIS.

26. The method according to claim 24, wherein the reference standard is a convertor reference standard from a subject who has CDMS.

27. The method according to any one of claims 24-26, wherein step d. comprises:
determining that the subject's prognosis is poor when:
i. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is increased when compared to the reference standard when the reference standard is a non-convertor reference standard; or
ii. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is decreased when compared to the reference standard when the reference standard is a non-convertor reference standard; or
iii. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is increased or the same when compared to the reference standard when the reference standard is a convertor reference standard; or
iv. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is decreased or the same when compared to the reference standard when the reference standard is a convertor reference standard; or
determining that the subject's prognosis is good when:
i. the measured concentration of one or more polypeptides selected from:
Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is decreased or the same when compared to the reference standard when the reference standard is a non-convertor reference standard; or
ii. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is increased or the same when compared to the reference standard when the reference standard is a non-convertor reference standard; or
iii. the measured concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein is decreased when compared to the reference standard when the reference standard is a convertor reference standard; or
iv. the measured concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 is increased when compared to the reference standard when the reference standard is a convertor reference standard.

28. The method according to any one of claims 24-27, wherein step d. comprises:
determining that the subject's prognosis is poor when:
i. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the measured concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
iv. the measured concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard; or
determining that the subject's prognosis is good when:
i. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
ii. the measured concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
iii. the measured concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
iv. the measured concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard.

29. The method according to any one of claims 24-28, further comprising measuring leukocyte concentration, mononuclear cell concentration, polynuclear cell concentration, serum albumin ratio and total protein concentration in a sample obtained from the subject.

30. The method according to claim 29, wherein the concentration of leukocytes, mononuclear and/or polynuclear cells in a sample obtained from the subject is:
i. decreased or the same when compared to a non-convertor reference standard and determines that a subject's prognosis is good;
ii. decreased when compared to a convertor reference standard and determines that a subject's prognosis is good;
iii. increased or the same when compared to a convertor reference standard and determines that a subject's prognosis is poor; or
iv. increased when compared to a non-convertor reference standard and determines that a subject's prognosis is poor.

31. The method according to claim 29 or 30, wherein the serum/albumin ratio and/or total protein concentration in a sample obtained from the subject is:
i. decreased or the same when compared to a convertor reference standard and determines that a subject's prognosis is poor;
ii. decreased when compared to a non-convertor reference standard and determines that a subject's prognosis is poor;
iii. increased or the same when compared to a non-convertor reference standard and determines that a subject's prognosis is good; or
iv. increased when compared to a convertor reference standard and determines that a subject's prognosis is good.

32. The method according to any one of the preceding claims, wherein at least one of the polypeptides is selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, and RING finger protein 165.

33. The method according to any one of the preceding claims, wherein at least one of the polypeptides is selected from: Ribosomal protein S6 kinase alpha-5, DNA repair protein XRCC1, Cytosolic acyl coenzyme A thioester hydrolase, Cytoplasmic tyrosine-protein kinase BMX, and Cathepsin K.

34. The method according to any one of the preceding claims, wherein at least one of the metabolites is selected from: creatinine, creatine, mobile lipoprotein (—CH2-)n resonances (VLDL and LDL), isoleucine, and glucose (serum).

35. The method according to any one of the preceding claims, wherein the concentration of the one or more metabolites is determined using a technique selected from: Nuclear Magnetic Resonance (NMR) spectroscopy, mass spectrometry, HPLC-UV, and infrared spectrometry, preferably wherein the NMR spectroscopy is ¹H-NMR spectroscopy.

36. The method according to any one of the preceding claims, wherein the concentration of the one or more polypeptides is determined indirectly by assessing gene expression.

37. The method according to claim 36, wherein gene expression is assessed by a technique selected from: transcriptomics, Northern blotting, quantitative reverse transcription (RT-PCR) and RNA sequencing (RNA-Seq).

38. The method according to any one of claims 1-35, wherein the concentration of the one or more polypeptides is determined directly by analysing polypeptide amounts/concentrations.

39. The method according to claim 38, wherein the polypeptide amounts/concentrations are analysed by a technique selected from: mass spectrometry, enzyme-linked immunosorbent assay (ELISA) and Luminex assay, more preferably wherein the concentration of the one or more polypeptides is determined directly by a SOMAscan Assay.

40. The method according to any one of the preceding claims, wherein the concentrations of at least 5 polypeptides are measured.

41. The method according to any one of the preceding claims, wherein the concentrations of at least 2 metabolites are measured.

42. The method according to any one of the preceding claims, wherein the concentrations of at least 5 polypeptides and the concentrations of at least 2 metabolites are measured.

43. The method according to any one of the preceding claims, wherein the one or more polypeptides comprise a polypeptide sequence having at least 70% sequence identity to any one of SEQ ID NOs: 1-91.

44. The method according to any one of the preceding claims, wherein the one or more polypeptides comprise a polypeptide sequence having at least 80% sequence identity to any one of SEQ ID NOs: 1-91.

45. The method according to any one of the preceding claims, wherein the one or more polypeptides comprise a polypeptide sequence having at least 90% sequence identity to any one of SEQ ID NOs: 1-91.

46. The method according to any one of the preceding claims, wherein the one or more polypeptides comprise a polypeptide sequence having at least 95% sequence identity to any one of SEQ ID NOs: 1-91.

47. The method according to any one of the preceding claims, wherein the one or more polypeptides comprise a polypeptide sequence comprising any one of SEQ ID NOs: 1-91.

48. The method according to any one of the preceding claims, wherein the biofluid sample is a cell-free biofluid sample.

49. The method according to claim 48, wherein the cell-free biofluid sample is not enriched for white blood cells.

50. The method according to claims 48 and 49, wherein the cell-free biofluid sample is not enriched for peripheral blood mononuclear cells (PBMCs), T-cells and/or monocytes.

51. The method according to any one of the preceding claims, wherein the sample is a biofluid sample selected from cerebrospinal fluid, blood (e.g. plasma), and urine.

52. The method according to any one of the preceding claims, wherein the convertor reference standard is a reference standard from a subject that has MS.

53. The method according to any one of the preceding claims, wherein the convertor reference standard is a reference standard from a subject that has CDMS.

54. The method according to any one of the preceding claims, wherein the convertor reference standard is a reference standard from a subject that has RRMS.

55. The method according to any one of the preceding claims, wherein the non-convertor reference standard is a reference standard from a subject that does not have MS (e.g. a healthy subject).

56. The method according to any one of the preceding claims, wherein the non-convertor reference standard is a reference standard from a subject that has CIS.

57. The method according to claim 56, wherein the non-convertor reference standard is from a subject that is a slow convertor.

58. A method, comprising:
a. obtaining a biofluid sample derived from a subject having, or suspected of having, CIS;
b. assaying the biofluid sample for a concentration of:
i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate.

59. The method of claim 58, further comprising:
c. comparing the assayed concentration with the concentration of the same one or more polypeptides and/or metabolites, respectively, in a reference standard; and
d. determining that the subject will convert from CIS to CDMS based on the comparison when:
i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
determining that the subject will not convert from CIS to CDMS based on the comparison when:
i. the assayed concentration of one or more polypeptides selected from:
Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
ii. the assayed concentration of one or more polypeptides selected from:
Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
iii. the assayed concentration of one or more polypeptides selected from:
Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
iv. the assayed concentration of one or more polypeptides selected from:
Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard.

60. A method for predicting whether a subject will convert from CIS to CDMS, the method comprising:
a. obtaining a biofluid sample derived from a subject having, or suspected of having, CIS;
b. assaying the biofluid sample for a concentration of:
i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the assayed concentration with the concentration of the same one or more polypeptides and/or metabolites, respectively, in a reference standard; and
d. determining that the subject will convert from CIS to CDMS based on the comparison when:
i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free increased or the same when compared to the reference standard when the reference standard is a convertor reference standard; or
iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
determining that the subject will not convert from CIS to CDMS based on the comparison when:
i. the assayed concentration of one or more polypeptides selected from:
Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard, thereby predicting whether a subject will convert from CIS to CDMS.

61. The method according to any one of claims 58-60, further comprising administering to the subject predicted to be a convertor a suitable therapeutic that delays conversion.

62. The method according to any one of claims 58-61, wherein the method predicts whether or not a subject will convert from CIS to CDMS within a period of 10 years, 5 years, or 4 years of CIS.

63. A method for diagnosing MS, the method comprising:
a. obtaining a biofluid sample derived from a subject;
b. assaying the biofluid sample for a concentration of:
i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2—CH2, glucose, glutamine, and lactate;
c. comparing the assayed concentration with the concentration of the same one or more polypeptides and/or metabolites, respectively, in a reference standard; and
d. diagnosing MS based on the comparison when:
i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
iv. the assayed concentration of one or more polypeptides selected from:
Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased or the same when compared to the reference standard when the reference standard is a convertor reference standard;
or
not diagnosing MS when:
v. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
vi. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
vii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
viii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard, thereby diagnosing or not diagnosing MS.

64. A method for predicting prognosis of MS, the method comprising:
a. obtaining a biofluid sample derived from a subject;
b. assaying the biofluid sample for a concentration of:
i. one or more polypeptides in the biofluid sample selected from Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, RING finger protein 165, Natural cytotoxicity triggering receptor 1, AP-1 complex subunit gamma-like 2, Prostaglandin reductase 1, Interleukin-5 receptor subunit alpha, Testis-specific serine/threonine-protein kinase 2, Tyrosine-protein kinase BLK, Lymphotoxin alpha2:beta1, Mitochondrial antiviral-signaling protein, GTP cyclohydrolase 1, Protein NOV homolog, Mitotic-spindle organizing protein 1, Guanine nucleotide exchange factor DBS, Vascular cell adhesion protein 1, Carbonyl reductase [NADPH] 1, Epididymal-specific lipocalin-10, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, ETS domain-containing protein Elk-1, Guanylyl cyclase-activating protein 1, Interferon regulatory factor 1, Beclin-1, Inositol polyphosphate 5-phosphatase OCRL-1, Dynein light chain Tctex-type 1, Thrombospondin-2, C—C motif chemokine 17, Dorsal root ganglia homeobox protein, Proenkephalin-A, T-lymphocyte surface antigen Ly-9, Muscle, skeletal receptor tyrosine-protein kinase, Myeloid zinc finger 1, Protein DGCR6, Tumor necrosis factor receptor superfamily member EDAR, Protocadherin alpha-7, High affinity immunoglobulin gamma Fc receptor I, CD40 ligand, Dickkopf-related protein 2, Growth-regulated alpha protein, Metalloproteinase inhibitor 2, Brother of CDO, BRISC complex subunit Abro1, Endoplasmic reticulum resident protein 44, Clumping factor B, Collagenase 3, Prokineticin-2, Peptidyl-prolyl cis-trans isomerase-like 2, Interleukin-22 receptor subunit alpha-2, Beta-sarcoglycan, Transmembrane glycoprotein NMB, Tumor necrosis factor receptor superfamily member 11B, Microfibril-associated glycoprotein 4, Collagen alpha-2(VI) chain, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Beta-defensin 123, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, Glial fibrillary acidic protein, and Allergin-1; and/or
ii. one or more metabolites in the biofluid sample selected from creatinine, creatine, mobile lipoprotein (—CH2-)n resonances (VLDL and LDL), isoleucine, leucine, mobile lipoprotein —CH3 resonances (HDL and LDL), betaine, mobile —N(CH3)3/free choline, formate, 3-hydroxybutyrate, myo-inositol, NAC1/=CH—CH2-CH2-, glucose, glutamine, and lactate;
c. comparing the assayed concentration with the concentration of the same one or more polypeptides and/or metabolites, respectively, in a reference standard; and
d. determining that the subject's prognosis is poor based on the comparison when:
i. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased when compared to the reference standard when the reference standard is a non-convertor reference standard; or
ii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is decreased when compared to the reference standard when the reference standard is a non-convertor reference standard; or
iii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is increased or the same when compared to the reference standard when the reference standard is a convertor reference standard; or
iv. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, when compared to the reference standard when the reference standard is a convertor reference standard;
or
determining that the subject's prognosis is good based on the comparison when:
v. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased or the same when compared to the reference standard when the reference standard is a non-convertor reference standard; or
vi. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased or the same when compared to the reference standard when the reference standard is a non-convertor reference standard; or
vii. the assayed concentration of one or more polypeptides selected from: Cytosolic acyl coenzyme A thioester hydrolase, Rho guanine nucleotide exchange factor 2, RING finger protein 165, Natural cytotoxicity triggering receptor 1, Interleukin-5 receptor subunit alpha, Lymphotoxin alpha2:beta1, Mitochondrial antiviral-signaling protein, GTP cyclohydrolase 1, Guanine nucleotide exchange factor DBS, Vascular cell adhesion protein 1, Epididymal-specific lipocalin-10, ETS domain-containing protein Elk-1, Beclin-1, Dynein light chain Tctex-type 1, C—C motif chemokine 17, T-lymphocyte surface antigen Ly-9, Myeloid zinc finger 1, Protein DGCR6, Growth-regulated alpha protein, Clumping factor B, Peptidyl-prolyl cis-trans isomerase-like 2, Interleukin-22 receptor subunit alpha-2, Beta-sarcoglycan, Transmembrane glycoprotein NMB, Collagen alpha-2(VI) chain, Beta-defensin 123, and Glial fibrillary acidic protein and/or the assayed concentration of one or more metabolites selected from: mobile lipoprotein (—CH2-)n resonances (VLDL and LDL), mobile lipoprotein —CH3 resonances (HDL and LDL), mobile —N(CH3)3/free choline, formate, glucose (CSF), NAC1/=CH—CH2-CH2-, and lactate is decreased when compared to the reference standard when the reference standard is a convertor reference standard; or
viii. the assayed concentration of one or more polypeptides selected from: Ribosomal protein S6 kinase alpha-5, DNA repair protein XRCC1, Cytoplasmic tyrosine-protein kinase BMX, Cathepsin K, Tropomyosin alpha-3 chain, Pleckstrin homology domain-containing family A member 1, Calcium uptake protein 2, mitochondrial, AP-1 complex subunit gamma-like 2, Prostaglandin reductase 1, Testis-specific serine/threonine-protein kinase 2, Tyrosine-protein kinase BLK, Protein NOV homolog, Mitotic-spindle organizing protein 1, Carbonyl reductase [NADPH] 1, Coiled-coil domain-containing protein 80, Grancalcin, Polypeptide N-acetylgalactosaminyltransferase 16, Complement factor H, Interleukin-32, RAF proto-oncogene serine/threonine-protein kinase, D-glucuronyl C5-epimerase, Proteasomal ubiquitin receptor ADRM1, Nuclear receptor subfamily 1 group D member 2, Beta-crystallin B2, Zinc finger protein 41, Guanylyl cyclase-activating protein 1, Interferon regulatory factor 1, Inositol polyphosphate 5-phosphatase OCRL-1, Thrombospondin-2, Dorsal root ganglia homeobox protein, Proenkephalin-A, Muscle, skeletal receptor tyrosine-protein kinase, Tumor necrosis factor receptor superfamily member EDAR, Protocadherin alpha-7, High affinity immunoglobulin gamma Fc receptor I, CD40 ligand, Dickkopf-related protein 2, Metalloproteinase inhibitor 2, Brother of CDO, BRISC complex subunit Abro1, Endoplasmic reticulum resident protein 44, Collagenase 3, Prokineticin-2, Tumor necrosis factor receptor superfamily member 11B, Microfibril-associated glycoprotein 4, RNA polymerase II elongation factor ELL2, EF-hand calcium-binding domain-containing protein 14, Essential MCU regulator (mitochondrial), Importin subunit alpha-1, Leucine-rich repeat-containing protein 3, V-set and immunoglobulin domain-containing protein 2, Serine protease inhibitor Kazal-type 13, Insulin-like growth factor-binding protein 1, Spermatogenesis-associated protein 9, Heterogeneous nuclear ribonucleoprotein K, Drebrin-like protein, Desert hedgehog protein N-product, Inhibin beta A chain:Inhibin beta B chain heterodimer, Retinoic acid receptor responder protein 2, Lutropin-choriogonadotropic hormone receptor, Thrombospondin-4, and Allergin-1 and/or the assayed concentration of one or more metabolites selected from: creatinine, creatine, isoleucine, leucine, betaine, 3-hydroxybutyrate, myo-inositol, glucose (serum), and glutamine is increased when compared to the reference standard when the reference standard is a convertor reference standard, thereby predicting the prognosis of MS.

65. The method according to any one of claims 58-64, wherein step b. comprises assaying the concentration of one or more metabolites and one or more polypeptides in the biofluid sample.

66. The method according to any one of claims 58-65, wherein at least one of the polypeptides is selected from: Ribosomal protein S6 kinase alpha-5, DNA repair protein XRCC1, Cytosolic acyl coenzyme A thioester hydrolase, Cytoplasmic tyrosine-protein kinase BMX, Cathepsin K, Tropomyosin alpha-3 chain, Rho guanine nucleotide exchange factor 2, Pleckstrin homology domain-containing family A member 1, Calcium uptake protein 2, mitochondrial, and RING finger protein 165.

67. The method according to any one of claims 58-66, wherein at least one of the polypeptides is selected from: Ribosomal protein S6 kinase alpha-5, DNA repair protein XRCC1, Cytosolic acyl coenzyme A thioester hydrolase, Cytoplasmic tyrosine-protein kinase BMX, and Cathepsin K.

68. The method according to any one of claims 58-67, wherein the concentration of the one or more polypeptides is assayed using a technique selected from: Northern blot, quantitative reverse transcription PCR (RT-PCR), RNA sequencing, transcriptomics, proteomics, mass-spectrometry, tandem mass-spectrometry, a gel-based technique, and differential in-gel electrophoresis.

69. The method according to any one of claims 58-68, wherein the concentration of the one or more metabolites is assayed using a technique selected from: Nuclear Magnetic Resonance (NMR) spectroscopy, mass spectrometry, HPLC-UV, and infrared spectrometry.

70. The method according to any one of claims 58-69 wherein the biofluid sample is a cell-free biofluid sample.

71. The method according to any one of claims 58-70, wherein the cell-free biofluid sample is not enriched for white blood cells.

72. The method according to claim 70 or 71, wherein the cell-free biofluid sample is not enriched for peripheral blood mononuclear cells (PBMCs), T-cells and/or monocytes.

73. The method according to any one of claims 58-72, wherein the biofluid sample is selected from cerebrospinal fluid, blood (e.g. plasma), and urine.

74. The method according to any one of claims 58-73, wherein the reference standard is a convertor reference standard.

75. The method according to claim 74, wherein the convertor reference standard is a reference standard from a subject that has MS.

76. The method according to claim 74 or 75, wherein the convertor reference standard is a reference standard from a subject that has CDMS.

77. The method according to claim 74 or 75, wherein the convertor reference standard is a reference standard from a subject that has RRMS.

78. The method according to any one of claims 58-73, wherein the reference standard is a non-convertor reference standard.

79. The method according to claim 78, wherein the non-convertor reference standard is a reference standard from a subject that does not have MS (e.g. a healthy subject).

80. The method according to claim 78 or 79, wherein the non-convertor reference standard is a reference standard from a subject that has CIS.

81. The method according to any one of the preceding claims, further comprising recording the output of at least one step on a data-storage medium.

82. A data-storage medium, comprising data obtained by the method according to any one of the preceding claims.

83. A device for use in the method according to any one of claims 1-81, wherein said device is capable of performing the step of identifying: a concentration difference of one or more polypeptides and/or one or more metabolites in the sample when compared to the reference standard.

84. A therapeutic for use in a method of treating MS in a subject, said method comprising:
a. obtaining the results of a method of the invention according to any one of claim 1-14, 32-62 or 65-81; and
b. administering a therapeutic for MS when it has been determined that a subject will convert from CIS to CDMS.

85. A therapeutic for use in a method of treating MS in a subject, said method comprising:
a. providing a sample obtained from the subject;
b. measuring a concentration of:
i. one or more polypeptides in the sample; and/or
ii. one or more metabolites in the sample;
c. comparing the measured concentration with the concentration of the same one or more polypeptides and/or metabolites, respectively, in a reference standard;
d. determining that the subject will convert from CIS to CDMS based on the comparison, or determining that the subject will not convert from CIS to CDMS based on the comparison; and
e. administering a therapeutic for MS when it has been determined that a subject will convert from CIS to CDMS.

86. A therapeutic for use in a method of treating MS in a subject, said method comprising:
a. obtaining the results of a method of the invention according to any one of claim 15-23, 32-57, 63 or 65-81; and
b. administering a therapeutic for MS when a subject is diagnosed as having MS.

87. A therapeutic for use in a method of treating MS in a subject, said method comprising:
a. providing a sample obtained from the subject;
b. measuring a concentration of:
i. one or more polypeptides in the sample; and/or
ii. one or more metabolites in the sample;
c. comparing the measured concentration with the concentration of the same one or more polypeptides and/or metabolites, respectively, in a reference standard;
d. diagnosing MS, or not diagnosing MS based on the comparison; and
e. administering a therapeutic for MS when a subject is diagnosed as having MS.

88. A therapeutic for use in a method of treating MS in a subject, said method comprising:
a. obtaining the results of a method of the invention according to any one of claim 24-57 or 64-81; and
b. administering a therapeutic for MS when a subject is determined to have a poor prognosis.

89. A therapeutic for use in a method of treating MS in a subject, said method comprising:
a. providing a sample obtained from the subject;
b. measuring a concentration of:
i. one or more polypeptides in the sample; and/or
ii. one or more metabolites in the sample;
c. comparing the measured concentration with the concentration of the same one or more polypeptides and/or metabolites, respectively, in a reference standard;
d. determining that the subject's prognosis is poor based on the comparison or determining that the subject's prognosis is good based on the comparison; and
e. administering a therapeutic for MS when a subject is determined to have a poor prognosis.
